(19) United States
(12) Patent Application Publication
(10) Pub. No.: US 2023/0119911 A1
(43) Pub. Date: Apr. 20, 2023

(54) ORGANIC LIGHT-EMITTING DEVICE

(71) Applicant: LG CHEM, LTD., Seoul (KR)

(72) Inventors: Jiyoung NOH, Daejeon (KR); Yongbum CHA, Daejeon (KR); Dong Uk HEO, Daejeon (KR); Woochul LEE, Daejeon (KR); Dongkeun SONG, Daejeon (KR); Jaegoo LEE, Daejeon (KR)

(21) Appl. No.: 17/797,832

(22) PCT Filed: Feb. 16, 2021
(86) PCT No.: PCT/KR2021/001961

§ 371 Date: Aug. 5, 2022

(30) Foreign Application Priority Data
Feb. 20, 2020 (KR) 10-2020-0021078

(51) Int. Cl. H01L 51/00; C07F 7/08; C09K 11/06; C07D 209/86; C07C 15/28; C07D 251/24; C07D 405/10

(52) U.S. Cl. CPC H01L 51/0061; C07F 7/081; C09K 11/06; H01L 51/0094; C07D 209/86; H01L 51/006; C07C 15/28; H01L 51/0058; C07D 251/24; H01L 51/0067; H01L 51/0052; C07D 405/10; H01L 51/0073; H01L 51/5016

(57) ABSTRACT

Provided is an organic light-emitting device including an anode; a cathode; a light-emitting layer between the anode and the cathode and including a compound of Chemical Formula 2:

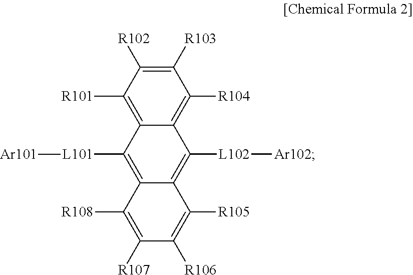

a first organic material layer between the anode and the light-emitting layer and including a compound of Chemical Formula 1:

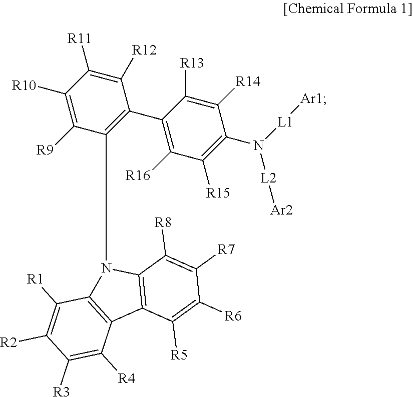

and a second organic material layer between the light-emitting layer and the cathode and including a compound of Chemical Formula 3:

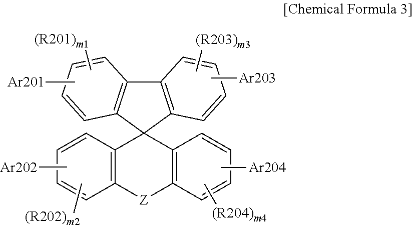

wherein:
Ar101, Ar102 and R101 to R108 are each independently hydrogen, deuterium, or a substituted or unsubstituted alkyl or aryl group;
Ar1 and Ar2 are each independently hydrogen, deuterium, a halogen, a cyano, or a substituted or unsubstituted silyl, alkyl, cycloalkyl, or aryl group;
R201 to R204 are each independently hydrogen, deuterium, a halogen, a cyano, or a substituted or unsubstituted alkyl, cycloalkyl, aryl group, or heterocyclic group; and
Z is O or S.

### CROSS-REFERENCE TO RELATED APPLICATIONS

This application is a National Stage Application of International Application No. PCT/KR2021/001961 filed on Feb. 16, 2021, which claims priority to and the benefits of Korean Patent Application No. 10-2020-0021078, filed with the Korean Intellectual Property Office on Feb. 20, 2020, the entire contents of which are incorporated herein by reference.

### TECHNICAL FIELD

The present specification relates to an organic light emitting device.

### BACKGROUND

An organic light emission phenomenon generally refers to a phenomenon converting electrical energy to light energy using an organic material. An organic light emitting device using an organic light emission phenomenon normally has a structure including an anode, a cathode, and an organic material layer therebetween. Herein, the organic material layer is often formed in a multilayer structure formed with different materials in order to increase efficiency and stability of the organic light emitting device, and for example, can be formed with a hole injection layer, a hole transfer layer, a light emitting layer, an electron transfer layer, an electron injection layer and the like. When a voltage is applied between the two electrodes in such an organic light emitting device structure, holes and electrons are injected to the organic material layer from the anode and the cathode, respectively, and when the injected holes and electrons meet, excitons are formed, and light emits when these excitons fall back to the ground state.

Development of new materials for such an organic light emitting device has been continuously required.

### PRIOR ART DOCUMENTS

### Patent Documents

(Patent Document 1) Korean Patent Application Laid-Open Publication No. 2016-132822

### Non-Patent Documents

(Non-Patent Document 1) A. D. Becke, Phys. Rev. A, 38, 3098 (1988)
(Non-Patent Document 2) J. P. Perdew and Y. Wang, Phys. Rev. B, 45, 13244 (1992)
(Non-Patent Document 3) B. Delley, J. Chem. Phys., 92, 508 (1990)
(Non-Patent Document 4) W. J. Hehre et al., J. Chem. Phys. 56, 2257 (1972)

### BRIEF DESCRIPTION

### Technical Problem

The present specification is directed to providing an organic light emitting device.

### Technical Solution

One embodiment of the present specification provides an organic light emitting device including an anode; a cathode; a light emitting layer provided between the anode and the cathode and including a compound of the following Chemical Formula 2; a first organic material layer provided between the anode and the light emitting layer and including a compound of the following Chemical Formula 1; and a second organic material layer provided between the light emitting layer and the cathode and including a compound of the following Chemical Formula 3:

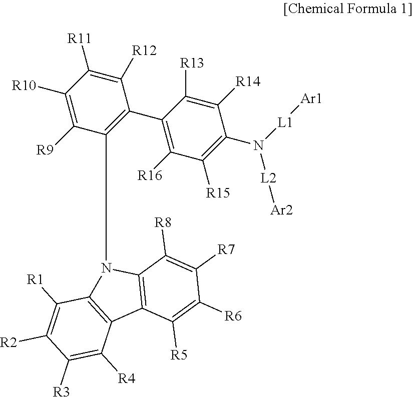

wherein in Chemical Formula 1:

L1 and L2 are the same as or different from each other, and each independently is a direct bond or a substituted or unsubstituted arylene group;

Ar1 and Ar2 are the same as or different from each other, and each independently is hydrogen, deuterium, a halogen group, a cyano group, a substituted or unsubstituted silyl group, a substituted or unsubstituted alkyl group, a substituted or unsubstituted cycloalkyl group, or a substituted or unsubstituted aryl group;

R1 to R8 are the same as or different from each other, and each independently is hydrogen, or deuterium, or bond to adjacent groups to form a substituted or unsubstituted ring; and

R9 to R16 are the same as or different from each other, and each independently is hydrogen or deuterium;

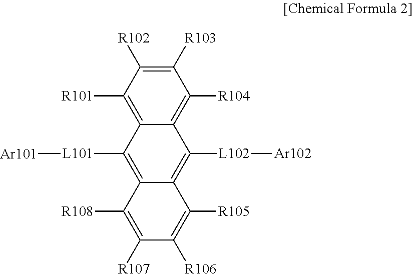

wherein in Chemical Formula 2:

L101 and L102 are the same as or different from each other, and each independently is a direct bond, a substituted or unsubstituted alkylene group, or a substituted or unsubstituted arylene group; and

Ar101, Ar102 and R101 to R108 are the same as or different from each other, and each independently is hydrogen, deuterium, a substituted or unsubstituted alkyl group, or a substituted or unsubstituted aryl group;

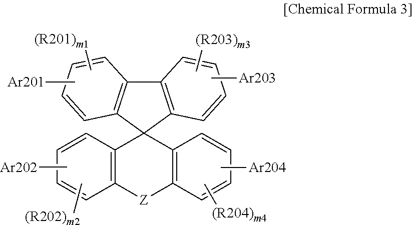

wherein in Chemical Formula 3:

Z is O or S;

R201 to R204 are the same as or different from each other, and each independently is hydrogen, deuterium, a halogen group, a cyano group, a substituted or unsubstituted alkyl group, a substituted or unsubstituted cycloalkyl group, a substituted or unsubstituted aryl group, or a substituted or unsubstituted heterocyclic group;

m1 to m4 are each an integer of 0 to 3, and when m1 to m4 are each 2 or greater, substituents in the parentheses are the same as or different from each other;

Ar201 and Ar202 are the same as or different from each other, at least one of Ar201 and Ar202 is -L201-CN, and any remaining is hydrogen;

L201 is a direct bond or a substituted or unsubstituted arylene group; and

Ar203 and Ar204 are the same as or different from each other, at least one of Ar203 and Ar204 is the following Chemical Formula A-1, and any remaining is hydrogen:

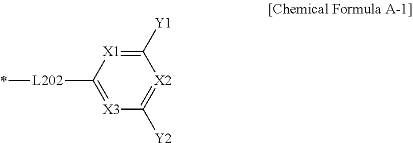

wherein in Chemical Formula A-1:

X1 to X3 are each N or CY3, and at least two of X1 to X3 are N;

Y1 to Y3 are the same as or different from each other, and each independently is hydrogen, deuterium, a substituted or unsubstituted alkyl group, a substituted or unsubstituted hydrocarbon ring group, or a substituted or unsubstituted heterocyclic group;

L202 is a direct bond or a substituted or unsubstituted arylene group; and

* means a bonding position.

### Advantageous Effects

An organic light emitting device described in the present specification includes a compound of Chemical Formula 2 in a light emitting layer, includes a compound of Chemical Formula 1 in a first organic material layer between an anode and the light emitting layer, and includes a compound of Chemical Formula 3 in a second organic material layer between the light emitting layer and a cathode, and as a result, an organic light emitting device having excellent light emission efficiency, low driving voltage, high efficiency and long lifetime can be obtained.

### DESCRIPTION OF DRAWINGS

FIG. 1 illustrates an organic light emitting device according to one embodiment of the present specification.

### REFERENCE NUMERALS

1: Substrate

2: Anode

3: Hole Injection Layer

4: Hole Transfer Layer

5: Electron Blocking Layer

6: Light Emitting Layer

7: Hole Blocking Layer

8: Electron Transfer Layer

9: Cathode

### DETAILED DESCRIPTION

Hereinafter, the present specification will be described in more detail.

One embodiment of the present specification provides an organic light emitting device including an anode; a cathode; a light emitting layer between the anode and the cathode and including a compound of Chemical Formula 2; a first organic material layer between the anode and the light emitting layer and including a compound of Chemical Formula 1; and a second organic material layer between the light emitting layer and the cathode and including a compound of Chemical Formula 3.

Adjusting a carrier balance of a light emitting layer is important in order to improve performance of an organic light emitting device, and by combining material of the light emitting layer and adjacent layers, a carrier balance in the light emitting layer can be adjusted.

By the organic light emitting device of the present specification using the compound of Chemical Formula 2, a material having enhanced hole injection and transfer properties, as a material of the light emitting layer, using the compound of Chemical Formula 1 having enhanced hole injection and electron blocking properties as a material of the first organic material layer, and using the compound of Chemical Formula 3 having enhanced electron injection and transfer properties as a material of the second organic material layer, a light emitting area is formed at an interface between the first organic material layer and the light emitting layer, and through an effect of increasing carrier density, a carrier balance is optimized resulting in a device with high efficiency.

In the present specification, a description of a certain part “including” certain constituents means capable of further including other constituents, and does not exclude other constituents unless particularly stated on the contrary.

Examples of substituents in the present specification are described below, however, the substituents are not limited thereto.

The team “substitution” means a hydrogen atom bonding to a carbon atom of a compound being changed to another substituent, and the position of substitution is not limited as long as it is a position at which a hydrogen atom is substituted, that is, a position at which a substituent can substitute, and when two or more substituents substitute, the two or more substituents can be the same as or different from each other.

In the present specification, the term “substituted or unsubstituted” means being substituted with one, two or more substituents selected from the group consisting of deuterium, a halogen group, a cyano group (—CN), a nitro group, a hydroxyl group, a carbonyl group, an ester group; an imide group, an amine group, a silyl group, a boron group, an alkoxy group, an alkyl group, a cycloalkyl group, an aryl group, and a heterocyclic group, or being substituted with a substituent linking two or more substituents among the substituents listed above, or having no substituents. For example, “a substituent linking two or more substituents” can include a biphenyl group. In other words, a biphenyl group can be an aryl group, or interpreted as a substituent linking two phenyl groups.

In addition, the term “substituted or unsubstituted” in the present specification means being substituted with one, two or more substituents selected from the group consisting of deuterium, a halogen group, a cyano group, an alkyl group, a cycloalkyl group, an aryl group, and a heterocyclic group, or being unsubstituted.

In the present specification, the halogen group can be fluorine, chlorine, bromine or iodine.

In the present specification, the alkyl group can be linear or branched, and although not particularly limited thereto, the number of carbon atoms is preferably from 1 to 30, more preferably from 1 to 20, and even more preferably from 1 to 10. Specific examples thereof can include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group and the like, but are not limited thereto.

In the present specification, the alkoxy group can be linear, branched or cyclic. The number of carbon atoms of the alkoxy group is not particularly limited, but is preferably from 1 to 30. Specific examples thereof can include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy and the like, but are not limited thereto.

In the present specification, the cycloalkyl group is not particularly limited, but preferably has 3 to 30 carbon atoms, and more preferably has 3 to 20 carbon atoms. Examples of the cycloalkyl group can include a cyclopropyl group, a cyclobutyl group, a cyclopentyl group, a 3-methylcyclopentyl group, a 2,3-dimethylcyclopentyl group, a cyclohexyl group, a cycloheptyl group, a cyclooctyl group and the like, but are not limited thereto.

In the present specification, a cycloalkenyl group is not particularly limited, but preferably has 3 to 30 carbon atoms. The cycloalkenyl group includes a double bond (—C═C—) in the cycloalkyl group described above.

In the present specification, the silyl group can be —Si(Ra)(Rb) (Rc)-, and Ra, Rb and Rc can be each independently hydrogen, a substituted or unsubstituted alkyl group having 1 to 30 carbon atoms, or a substituted or unsubstituted aryl group having 6 to 30 carbon atoms, but are not limited thereto. The silyl group can be an alkylsilyl group or an arylsilyl group, and furthermore, can be a trialkylsilyl group or a triarylsilyl group. The number of carbon atoms of the silyl group is not particularly limited, but is preferably from 1 to 30, and the number of carbon atoms of the alkylsilyl group can be from 1 to 30 and the number of carbon atoms of the arylsilyl group can be from 6 to 30. Specific examples thereof can include a trimethylsilyl group, a triethylsilyl group, a tert-butyldimethylsilyl group, a vinyldimethylsilyl group, a propyldimethylsilyl group, a triphenylsilyl group, a diphenylsilyl group, a phenylsilyl group and the like, but are not limited thereto.

In the present specification, the boron group can be —B(Rd) (Re), and Rd and Re can be each independently selected from the group consisting of hydrogen, deuterium, halogen, a nitrile group, a substituted or unsubstituted cycloalkyl group having 3 to 30 carbon atoms, a substituted or unsubstituted alkyl group having 1 to 30 carbon atoms, a substituted or unsubstituted aryl group having 6 to 30 carbon atoms, and a substituted or unsubstituted heteroaryl group having 2 to 30 carbon atoms, but are not limited.

In the present specification, the amine group can be —N(Rf) (Rg), and Rf and Rg can be each independently hydrogen, deuterium, a substituted or unsubstituted alkyl group having 1 to 30 carbon atoms, a substituted or unsubstituted aryl group having 6 to 30 carbon atoms, or a substituted or unsubstituted heteroaryl group having 2 to 30 carbon atoms, but are not limited thereto. Depending on the substituents bonding to Rf and Rg, the amine group can be —NH₂, an alkylamine group, an alkylarylamine group, an arylamine group, an arylheteroarylamine group, an alkylheteroarylamine group, and a heteroarylamine group. Specific examples of the amine group can include a methylamine group, a dimethylamine group, an ethylamine group, a diethylamine group, a phenylamine group, a naphthylamine group, a biphenylamine group, an anthracenylamine group, a 9-methyl-anthracenylamine group, a diphenylamine group, a phenylnaphthylamine group, an N-phenyltolylamine group, a triphenylamine group and the like, but are not limited thereto.

In the present specification, the aryl group is not particularly limited, but preferably has 6 to 60 carbon atoms, for example, 6 to 30 carbon atoms and more preferably 6 to 20 carbon atoms, and the aryl group can be monocyclic or polycyclic. When the aryl group is a monocyclic aryl group, the number of carbon atoms is not particularly limited, but is preferably from 6 to 60. Specific examples of the monocyclic aryl group can include a phenyl group, a biphenyl group, a terphenyl group and the like, but are not limited thereto.

When the aryl group is a polycyclic aryl group, the number of carbon atoms is not particularly limited, but is preferably from 10 to 60. Specific examples of the polycyclic aryl group can include a naphthyl group, an anthracenyl group, a phenanthryl group, a triphenyl group, a pyrenyl group, a phenalenyl group, a perylenyl group, a chrysenyl group, a fluorenyl group, a fluoranthenyl group and the like, but are not limited thereto.

In the present specification, the fluorenyl group being substituted includes two substituents that bond to the No. 9 carbon atom of the fluorene bonding to each other to form a ring, and examples thereof can include

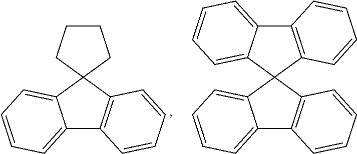

and the like. However, the structure is not limited thereto.

In the present specification, the heterocyclic group is a group including one or more atoms that are not carbon, that is, heteroatoms, and specifically, the heteroatom can include one or more atoms selected from the group consisting of O, N, S, Si, P and the like. The number of carbon atoms is not particularly limited, but is preferably from 1 to 60, furthermore, from 2 to 60, and the heterocyclic group can be a monocyclic or polycyclic. The heterocyclic group can be an aromatic ring, an aliphatic ring and a fused ring thereof. Examples of the heterocyclic group can include a thiophene group, a furanyl group, a pyrrole group, an imidazolyl group, a triazolyl group, an oxazolyl group, an oxadiazolyl group, a pyridyl group, a bipyridyl group, a pyrimidyl group, a triazinyl group, a triazolyl group, an acridyl group, a pyridazinyl group, a pyrazinyl group, a quinolyl group, a quinazolyl group, a quinoxalyl group, a phthalazinyl group, a pyridopyrimidyl group, a pyridopyrazinyl group, a pyrazinopyrazinyl group, an isoquinolyl group, an indolyl group, a carbazolyl group, a benzoxazolyl group, a benzimidazolyl group, a benzothiazolyl group, a benzocarbazolyl group, a benzothiophenyl group, a dibenzothiophenyl group, a benzofuranyl group, a phenanthrolinyl group, an isoxazolyl group, a thiadiazolyl group, a phenothiazinyl group, a dibenzofuranyl group and the like, but are not limited thereto.

The heteroaryl group means a monovalent aromatic heterocyclic group, and the descriptions on the heterocyclic group provided above can be applied thereto except for being aromatic.

In the present specification, the hydrocarbon ring group can be aromatic, aliphatic, or a fused ring group of aromatic and aliphatic.

In the present specification, the descriptions on the aryl group provided above can be applied to the aromatic hydrocarbon ring except for those that are a divalent group.

In the present specification, the aliphatic hydrocarbon ring means all hydrocarbon rings other than the aromatic hydrocarbon ring, and can include a cycloalkyl ring and a cycloalkene ring. The descriptions on the cycloalkyl group provided above can be applied to the cycloalkyl ring except for those that are a divalent group, and the descriptions on the cycloalkenyl group can be applied to the cycloalkene ring except for those that are a divalent group.

In the present specification, the “adjacent” group can mean a substituent substituting an atom directly linked to an atom substituted by the corresponding substituent, a substituent sterically most closely positioned to the corresponding substituent, or another substituent substituting an atom substituted by the corresponding substituent. For example, two substituents substituting ortho positions in a benzene ring, and two substituents substituting the same carbon in an aliphatic ring can be interpreted as groups “adjacent” to each other.

In the present specification, the “ring” in the substituted or unsubstituted ring formed by bonding to each other means a hydrocarbon ring or a heteroring. The descriptions on the hydrocarbon ring group provided above can be applied to the hydrocarbon ring except for those that are divalent. The descriptions on the heterocyclic group can be applied to the heteroring except for those that are divalent.

In the present specification, the descriptions on the aryl group provided above can be applied to the arylene group except for those that are a divalent group.

In the present specification, the descriptions on the alkyl group provided above can be applied to the alkylene group except for those that are a divalent group.

The organic light emitting device of the present specification includes a first organic material layer between an anode and a light emitting layer, and the first organic material layer includes the compound of Chemical Formula 1.

Hereinafter, Chemical Formula 1 will be described.

According to one embodiment of the present specification, R1 to R8 are the same as or different from each other, and each independently is hydrogen or deuterium, or bond to adjacent groups to form a substituted or unsubstituted ring.

According to one embodiment of the present specification, R1 to R8 are the same as or different from each other, and each independently is hydrogen or deuterium, or bond to adjacent groups to form a substituted or unsubstituted aromatic hydrocarbon ring.

According to one embodiment of the present specification, R1 to R8 are the same as or different from each other, and each independently is hydrogen or deuterium, or bond to adjacent groups to form a substituted or unsubstituted aromatic hydrocarbon ring having 6 to 30 carbon atoms.

R1 and R2, R2 and R3, or R3 and R4 among R1 to R8 bond to each other to form a substituted or unsubstituted aromatic hydrocarbon ring having 6 to 30 carbon atoms.

According to another embodiment, R1 to R8 are the same as or different from each other, and each independently is hydrogen or deuterium, or bond to adjacent groups to form a substituted or unsubstituted benzene ring.

According to one embodiment of the present specification, Chemical Formula 1 is any one of the following Chemical Formulae 1-1 to 1-4:

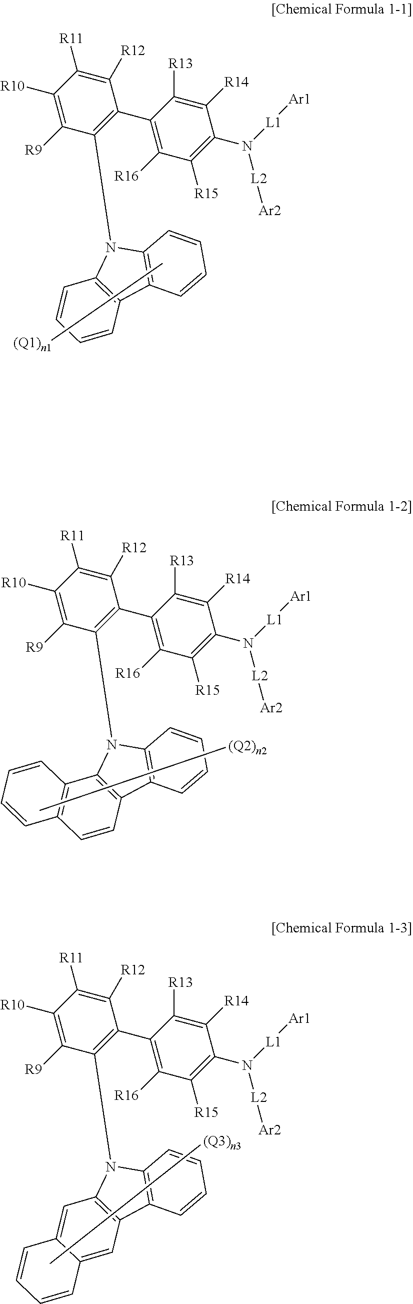

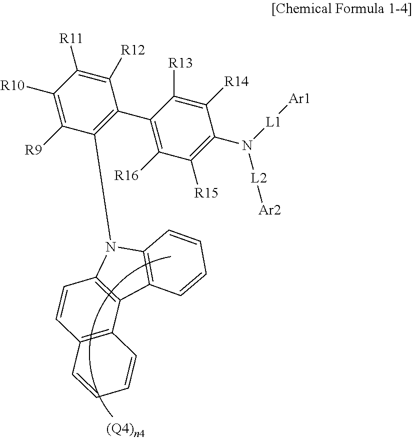

wherein in Chemical Formulae 1-1 to 1-4:

R9 to R16, L1, L2, Ar1 and Ar2 have the same definitions as in Chemical Formula 1;

Q1 to Q4 are the same as or different from each other, and each independently is hydrogen or deuterium;

n1 is an integer of 0 to 8, and n2 to n4 are each an integer of 0 to 10; and

when n1 to n4 are each 2 or greater, substituents in the parentheses are the same as or different from each other.

According to one embodiment of the present specification, Q1 to Q4 are all hydrogen.

According to one embodiment of the present specification, Q1 to Q4 are all deuterium.

According to one embodiment of the present specification, n1 is an integer of 0 to 8, and when n1 is 2 or greater, the two or more Q1s are the same as or different from each other.

According to one embodiment of the present specification, n2 is an integer of 0 to 10, and when n2 is 2 or greater, the two or more Q2s are the same as or different from each other.

According to one embodiment of the present specification, n3 is an integer of 0 to 10, and when n3 is 2 or greater, the two or more Q3s are the same as or different from each other.

According to one embodiment of the present specification, n4 is an integer of 0 to 10, and when n4 is 2 or greater, the two or more Q4s are the same as or different from each other.

According to one embodiment of the present specification, R9 to R16 are all hydrogen.

According to one embodiment of the present specification, R9 to R16 are all deuterium.

According to one embodiment of the present specification, L1 and L2 are the same as or different from each other, and each independently is a direct bond or a substituted or unsubstituted arylene group.

According to another embodiment, L1 and L2 are the same as or different from each other, and each independently is a direct bond or a substituted or unsubstituted arylene group having 6 to 60 carbon atoms.

According to another embodiment, L1 and L2 are the same as or different from each other, and each independently is a direct bond or a substituted or unsubstituted arylene group having 6 to 30 carbon atoms.

According to another embodiment, L1 and L2 are the same as or different from each other, and each independently is a direct bond, a substituted or unsubstituted phenylene group, a substituted or unsubstituted biphenylylene group, or a substituted or unsubstituted fluorenylene group.

According to another embodiment, L1 and L2 are the same as or different from each other, and each independently is a direct bond, a phenylene group, a biphenylylene group, or a fluorenylene group that is unsubstituted or substituted with a methyl group.

According to one embodiment of the present specification, Ar1 and Ar2 are the same as or different from each other, and each independently is hydrogen, deuterium, a halogen group, a cyano group, a substituted or unsubstituted silyl group having 1 to 30 carbon atoms, a substituted or unsubstituted alkyl group having 1 to 30 carbon atoms, a substituted or unsubstituted cycloalkyl group having 3 to 30 carbon atoms, or a substituted or unsubstituted aryl group having 6 to 60 carbon atoms.

According to another embodiment, Ar1 and Ar2 are the same as or different from each other, and each independently is hydrogen, deuterium, a substituted or unsubstituted phenyl group, a substituted or unsubstituted biphenyl group, a substituted or unsubstituted terphenyl group, a substituted or unsubstituted fluorenyl group, a substituted or unsubstituted naphthyl group, a substituted or unsubstituted phenanthryl group, or a substituted or unsubstituted triphenylenyl group.

According to another embodiment, Ar1 and Ar2 are the same as or different from each other, and each independently is hydrogen; deuterium; a phenyl group that is unsubstituted or substituted with one or more substituents selected from the group consisting of deuterium, a halogen group, a cyano group, a silyl group, an alkyl group and an aryl group; a biphenyl group that is unsubstituted or substituted with one or more substituents selected from the group consisting of deuterium, a halogen group, a cyano group, a silyl group, an alkyl group and an aryl group; a terphenyl group that is unsubstituted or substituted with one or more substituents selected from the group consisting of deuterium, a halogen group, a cyano group, a silyl group, an alkyl group and an aryl group; a fluorenyl group that is unsubstituted or substituted with one or more substituents selected from the group consisting of deuterium, a halogen group, a cyano group, a silyl group, an alkyl group and an aryl group; a naphthyl group that is unsubstituted or substituted with one or more substituents selected from the group consisting of deuterium, a halogen group, a cyano group, a silyl group, an alkyl group and an aryl group; a phenanthryl group that is unsubstituted or substituted with one or more substituents selected from the group consisting of deuterium, a halogen group, a cyano group, a silyl group, an alkyl group and an aryl group; or a triphenylenyl group that is unsubstituted or substituted with one or more substituents selected from the group consisting of deuterium, a halogen group, a cyano group, a silyl group, an alkyl group and an aryl group.

According to another embodiment, Ar1 and Ar2 are the same as or different from each other, and each independently is hydrogen; deuterium; a phenyl group that is unsubstituted or substituted with one or more substituents selected from the group consisting of deuterium, a halogen group, a cyano group, a silyl group, an alkyl group and an aryl group; a biphenyl group that is unsubstituted or substituted with one or more substituents selected from the group consisting of deuterium, a halogen group, a cyano group, a silyl group, an alkyl group and an aryl group; a terphenyl group that is unsubstituted or substituted with one or more substituents selected from the group consisting of deuterium, a halogen group, a cyano group, a silyl group, an alkyl group and an aryl group; a dimethylfluorenyl group that is unsubstituted or substituted with one or more substituents selected from the group consisting of deuterium, a halogen group, a cyano group, a silyl group, an alkyl group and an aryl group; a diphenylfluorenyl group that is unsubstituted or substituted with one or more substituents selected from the group consisting of deuterium, a halogen group, a cyano group, a silyl group, an alkyl group and an aryl group; a spirobifluorenyl group that is unsubstituted or substituted with one or more substituents selected from the group consisting of deuterium, a halogen group, a cyano group, a silyl group, an alkyl group and an aryl group; a naphthyl group that is unsubstituted or substituted with one or more substituents selected from the group consisting of deuterium, a halogen group, a cyano group, a silyl group, an alkyl group and an aryl group; a phenanthryl group that is unsubstituted or substituted with one or more substituents selected from the group consisting of deuterium, a halogen group, a cyano group, a silyl group, an alkyl group and an aryl group; or a triphenylenyl group that is unsubstituted or substituted with one or more substituents selected from the group consisting of deuterium, a halogen group, a cyano group, a silyl group, an alkyl group and an aryl group.

According to another embodiment, Ar1 and Ar2 are the same as or different from each other, and each independently is a phenyl group, a biphenyl group, a terphenyl group, or a fluorenyl group substituted with a phenyl group.

According to one embodiment of the present specification, -L1-Ar1 and -L2-Ar2 are different from each other.

According to one embodiment of the present specification, the compound of Chemical Formula 1 is any one compound selected from among the following compounds:

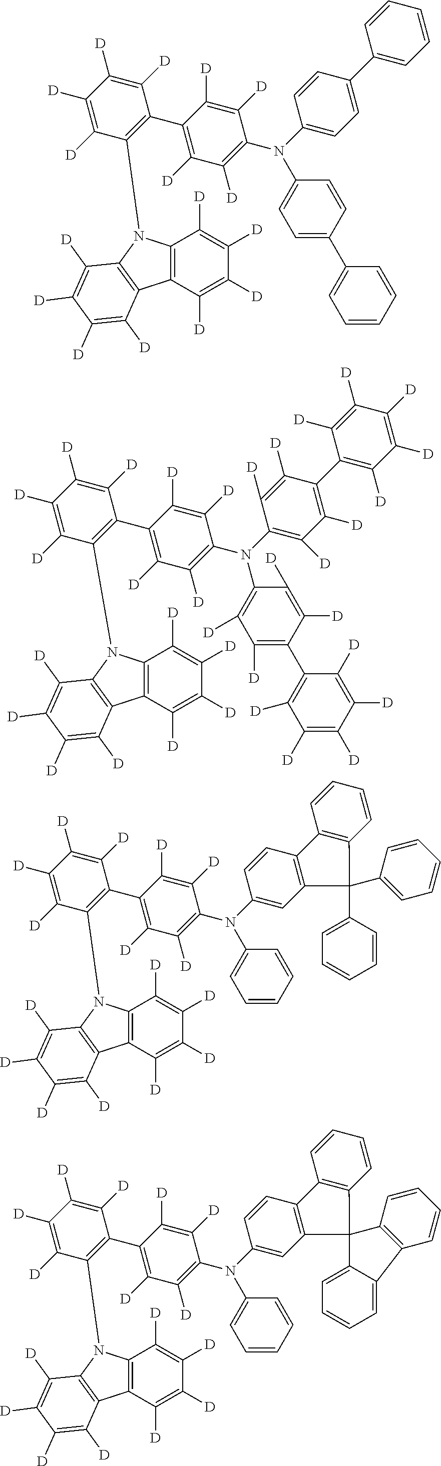

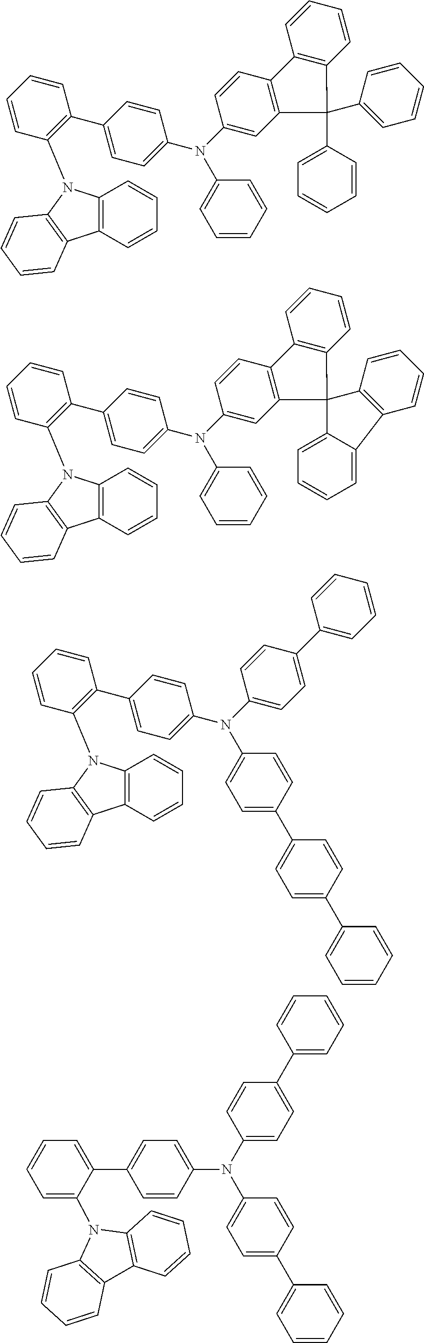

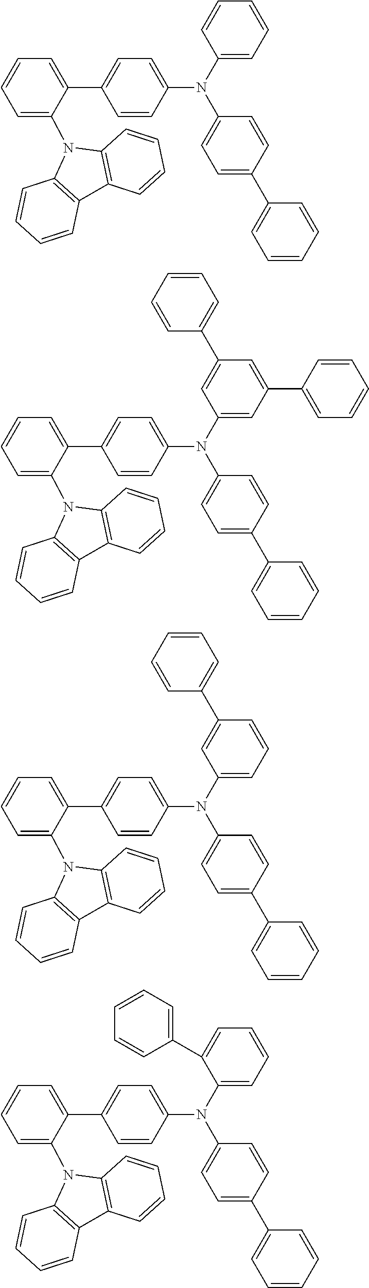 [SMILES: c1ccc(-c2ccc(N(c3ccccc3)c3ccc(-c4ccccc4-n4c5ccccc5c5ccccc54)cc3)cc2)cc1 ; c1ccc(-c2ccc(N(c3ccc(-c4ccccc4-n4c5ccccc5c5ccccc54)cc3)c3cc(-c4ccccc4)cc(-c4ccccc4)c3)cc2)cc1 ; c1ccc(-c2ccc(N(c3ccc(-c4ccccc4-n4c5ccccc5c5ccccc54)cc3)c3cccc(-c4ccccc4)c3)cc2)cc1 ; c1ccc(-c2ccc(N(c3ccc(-c4ccccc4-n4c5ccccc5c5ccccc54)cc3)c3ccccc3-c3ccccc3)cc2)cc1]

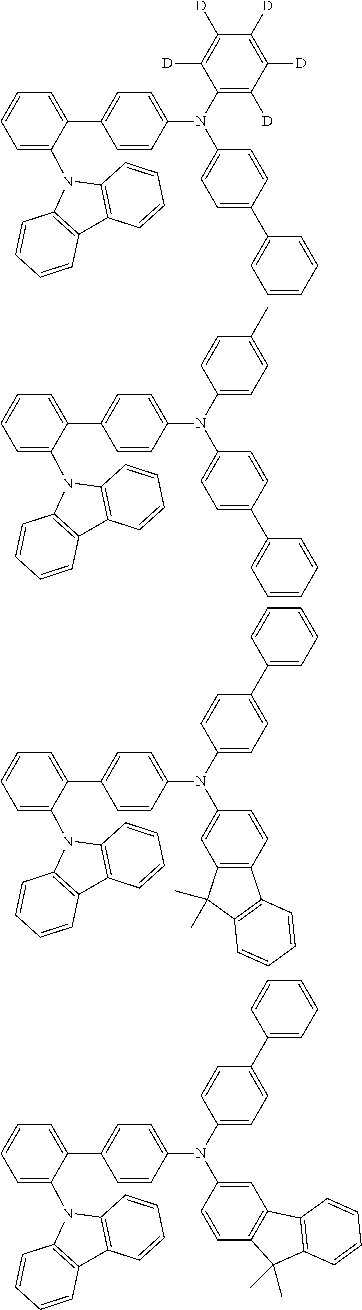

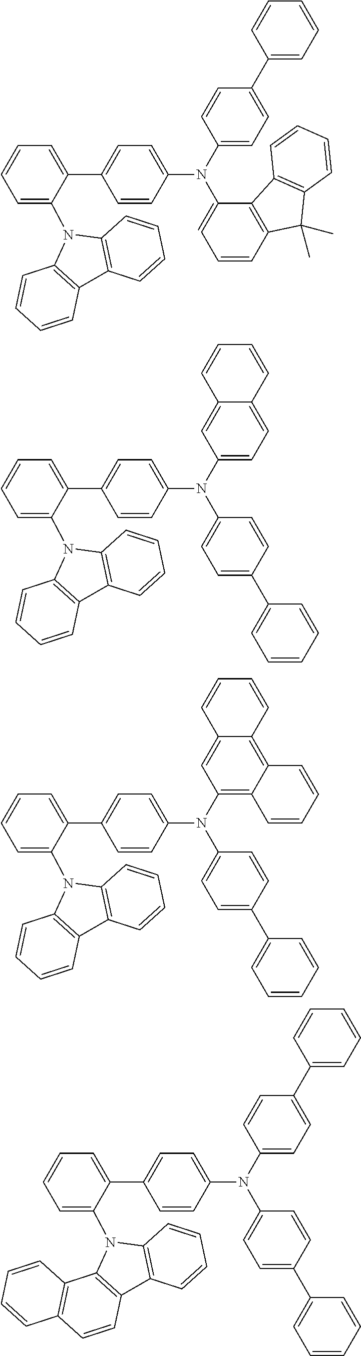

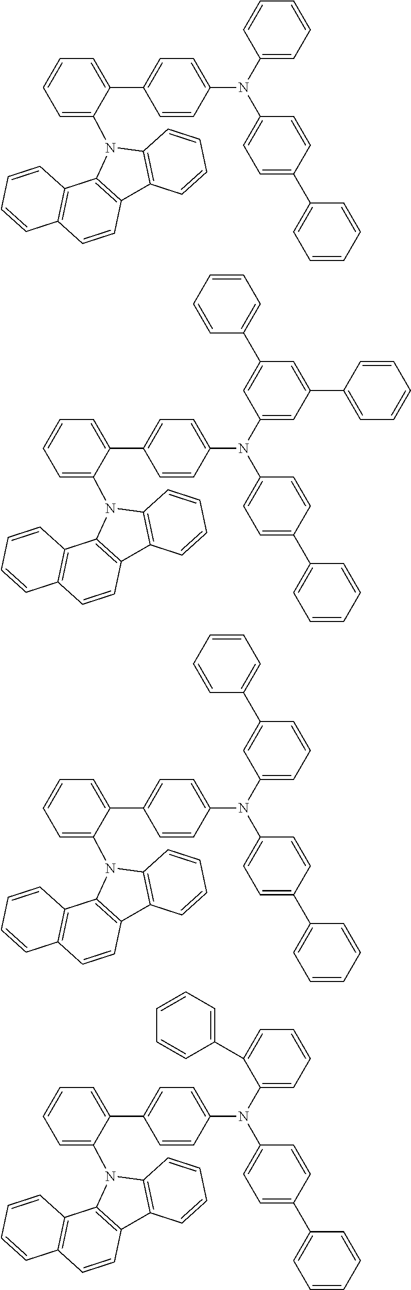 [SMILES: c1ccc(-c2ccc(N(c3ccccc3)c3ccc(-c4ccccc4-n4c5ccccc5c5ccc6ccccc6c54)cc3)cc2)cc1 ; c1ccc(-c2ccc(N(c3ccc(-c4ccccc4-n4c5ccccc5c5ccc6ccccc6c54)cc3)c3cc(-c4ccccc4)cc(-c4ccccc4)c3)cc2)cc1 ; c1ccc(-c2ccc(N(c3ccc(-c4ccccc4-n4c5ccccc5c5ccc6ccccc6c54)cc3)c3cccc(-c4ccccc4)c3)cc2)cc1 ; c1ccc(-c2ccc(N(c3ccc(-c4ccccc4-n4c5ccccc5c5ccc6ccccc6c54)cc3)c3ccccc3-c3ccccc3)cc2)cc1]

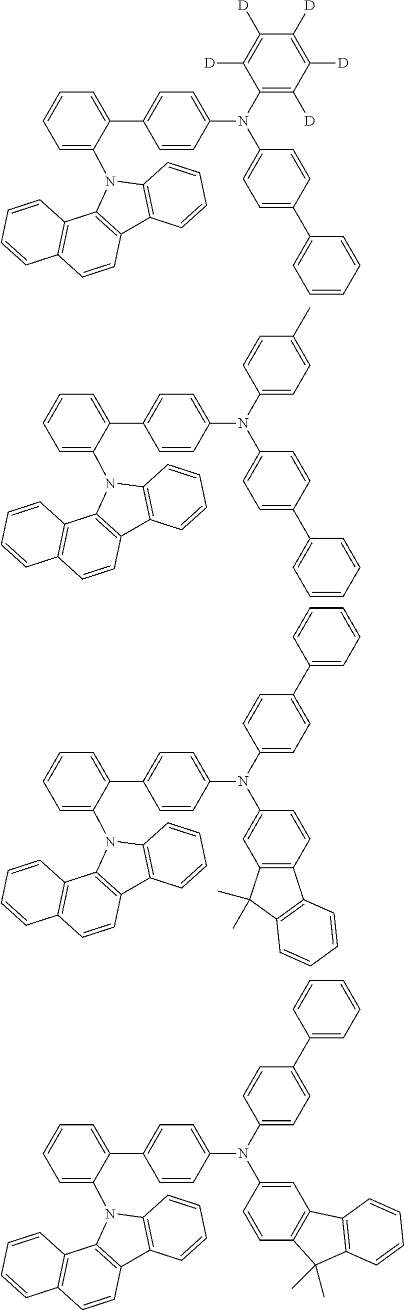

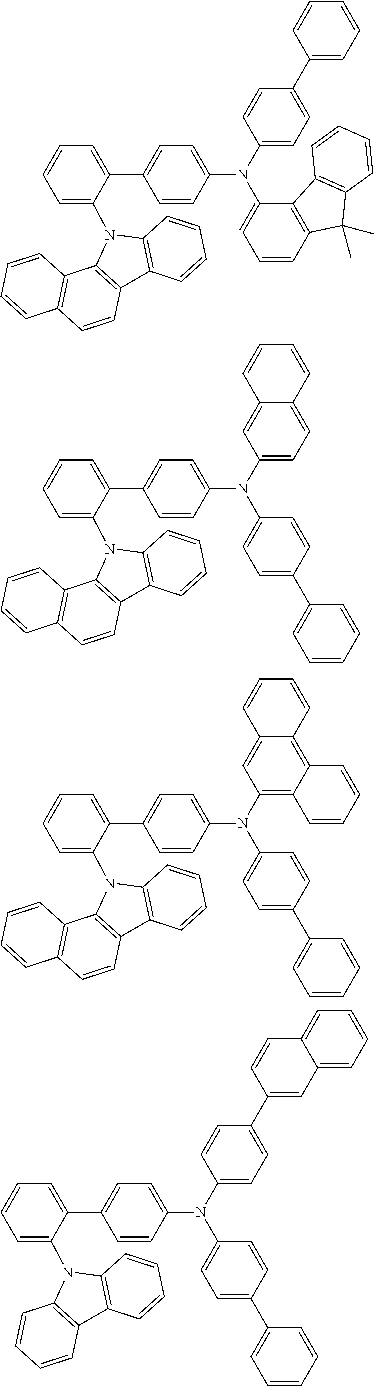

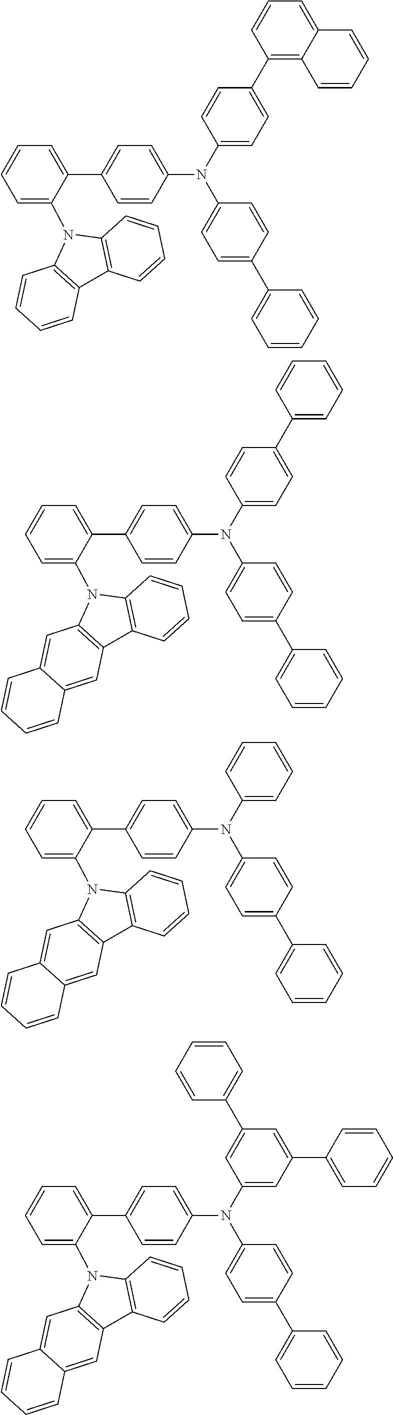

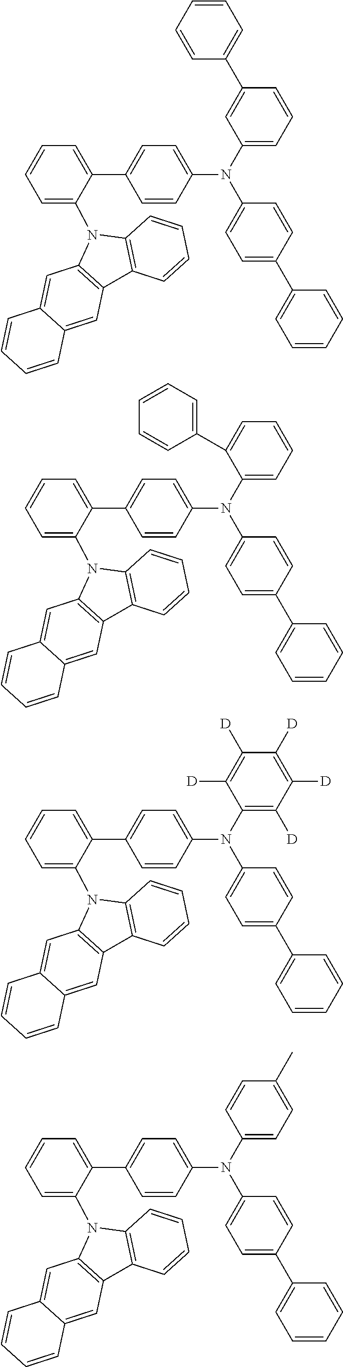

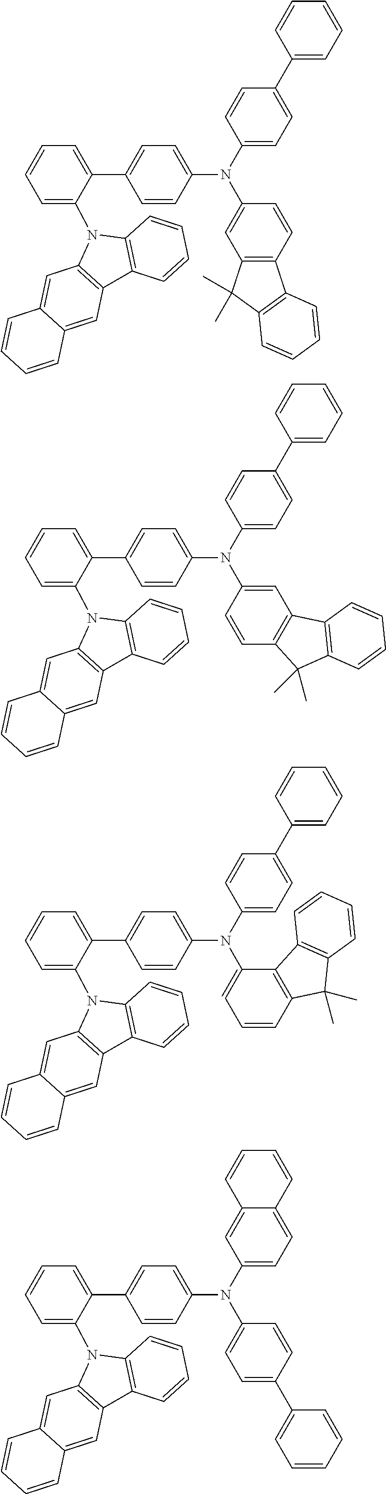

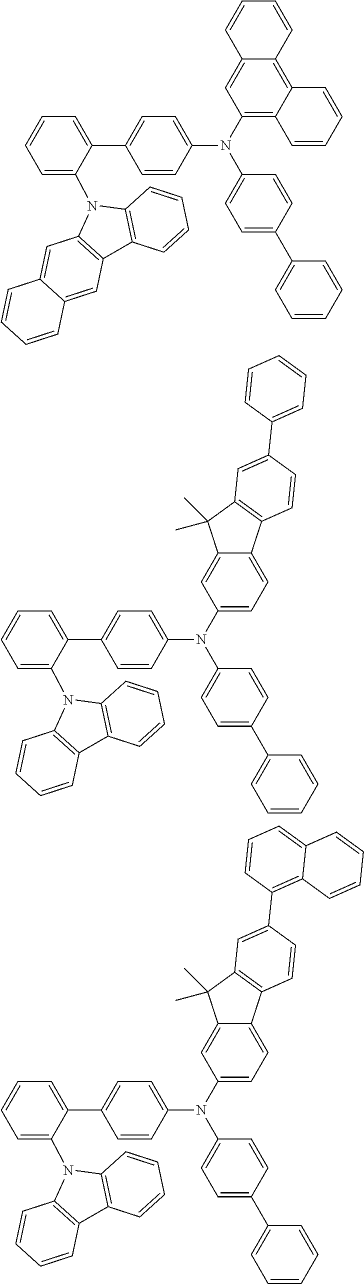

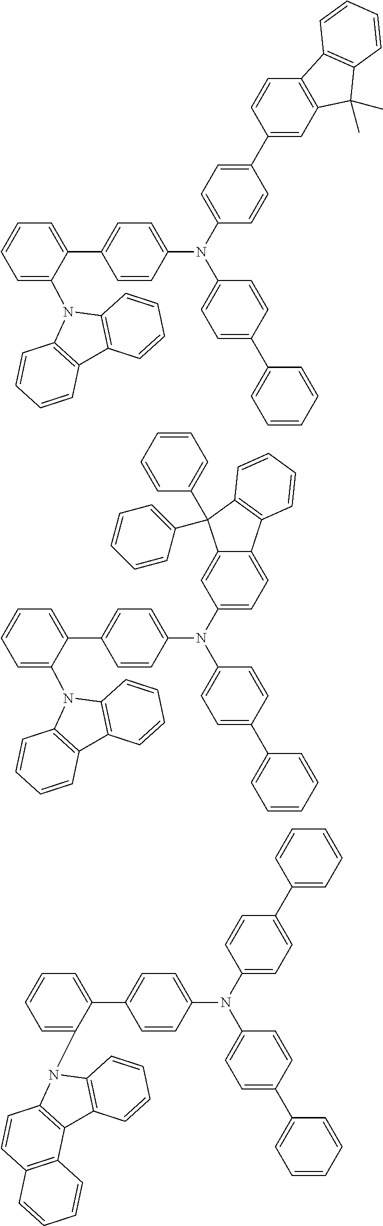

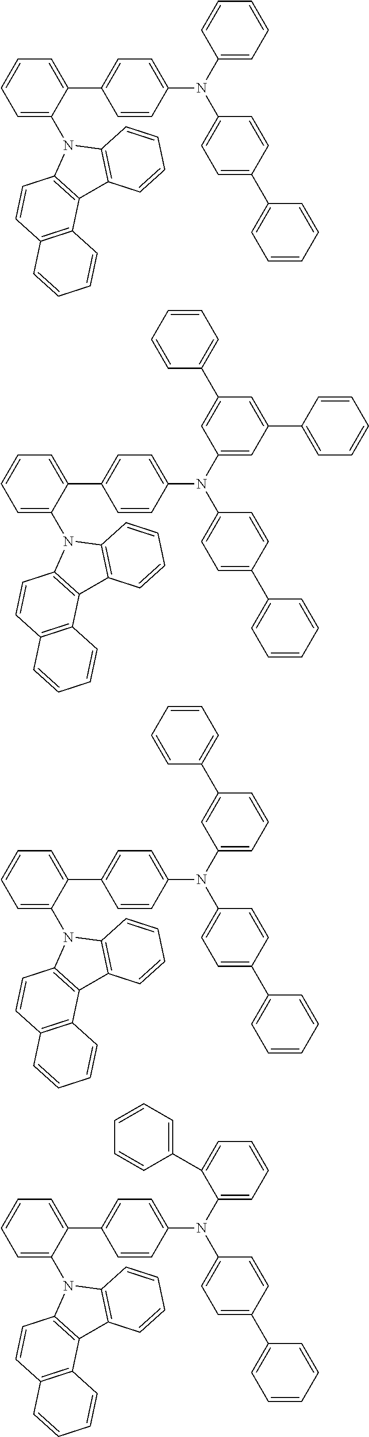 [SMILES: c1ccc(-c2ccc(N(c3ccccc3)c3ccc(-c4ccccc4-n4c5ccccc5c5c6ccccc6ccc54)cc3)cc2)cc1 ; c1ccc(-c2ccc(N(c3ccc(-c4ccccc4-n4c5ccccc5c5c6ccccc6ccc54)cc3)c3cc(-c4ccccc4)cc(-c4ccccc4)c3)cc2)cc1 ; c1ccc(-c2ccc(N(c3ccc(-c4ccccc4-n4c5ccccc5c5c6ccccc6ccc54)cc3)c3cccc(-c4ccccc4)c3)cc2)cc1 ; c1ccc(-c2ccc(N(c3ccc(-c4ccccc4-n4c5ccccc5c5c6ccccc6ccc54)cc3)c3ccccc3-c3ccccc3)cc2)cc1]

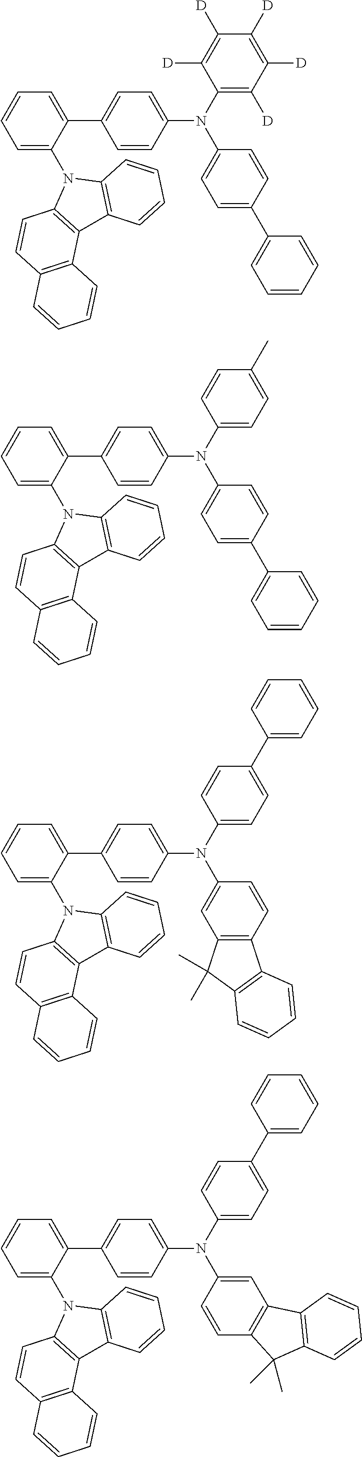

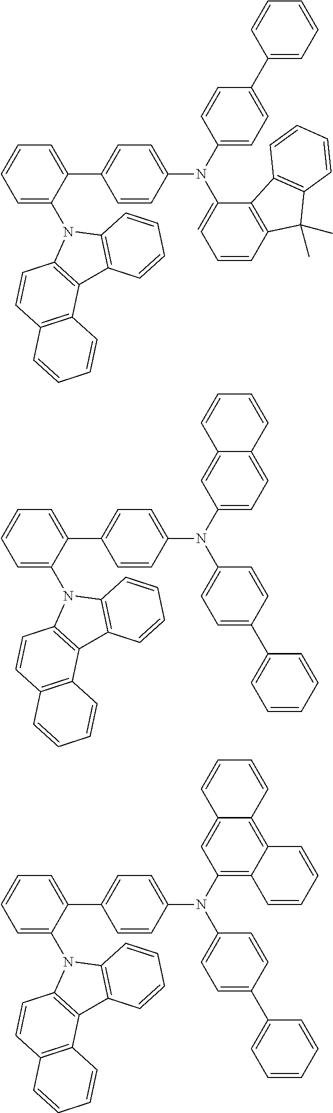 [SMILES: CC1(C)c2ccccc2-c2c(N(c3ccc(-c4ccccc4)cc3)c3ccc(-c4ccccc4-n4c5ccccc5c5c6ccccc6ccc54)cc3)cccc21 ; c1ccc(-c2ccc(N(c3ccc(-c4ccccc4-n4c5ccccc5c5c6ccccc6ccc54)cc3)c3ccc4ccccc4c3)cc2)cc1 ; c1ccc(-c2ccc(N(c3ccc(-c4ccccc4-n4c5ccccc5c5c6ccccc6ccc54)cc3)c3cc4ccccc4c4ccccc34)cc2)cc1]

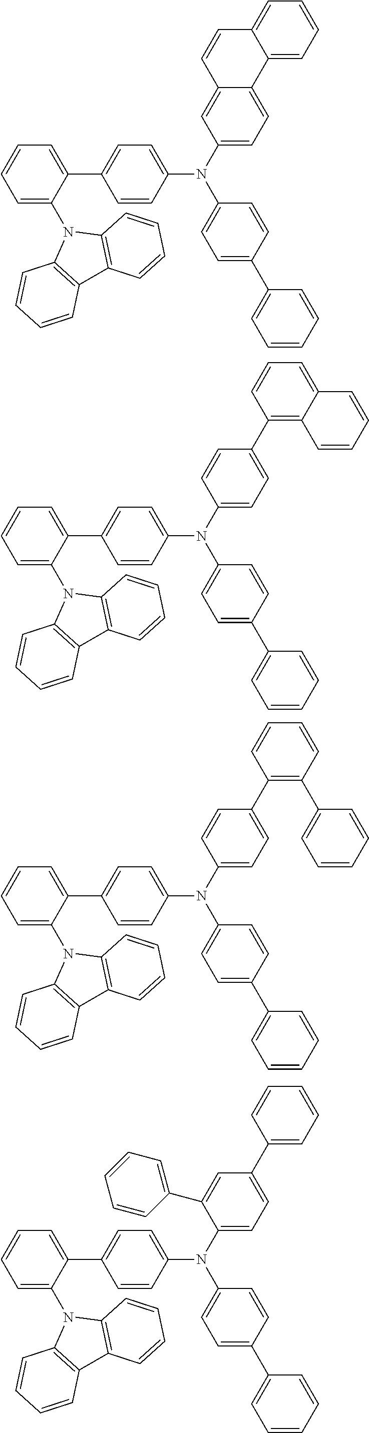

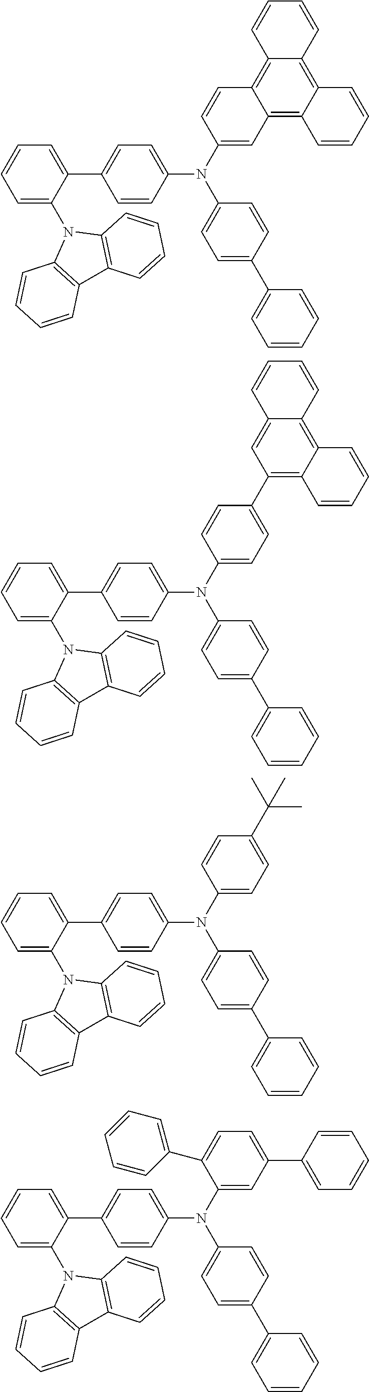

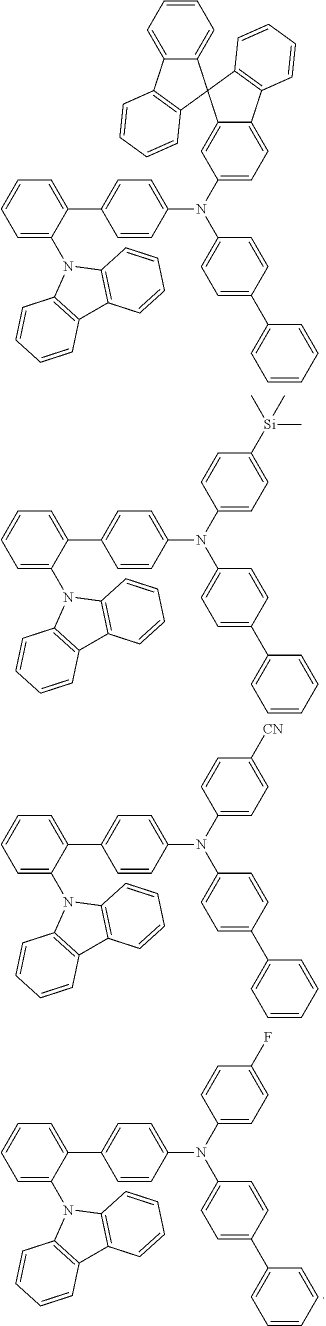

The organic light emitting device of the present specification includes a light emitting layer, and the light emitting layer includes the compound of Chemical Formula 2.

Hereinafter, Chemical Formula 2 will be described.

According to one embodiment of the present specification, L101 and L102 are the same as or different from each other, and each independently is a direct bond, a substituted or unsubstituted alkylene group having 1 to 30 carbon atoms, or a substituted or unsubstituted arylene group having 6 to 60 carbon atoms.

According to another embodiment, L101 and L102 are the same as or different from each other, and each independently is a direct bond, a substituted or unsubstituted alkylene group having 1 to 10 carbon atoms, or a substituted or unsubstituted arylene group having 6 to 30 carbon atoms.

According to another embodiment, L101 and L102 are the same as or different from each other, and each independently is a direct bond or a substituted or unsubstituted phenylene group.

According to another embodiment, L101 and L102 are the same as or different from each other, and each independently is a direct bond or a phenylene group that is unsubstituted or substituted with deuterium.

According to another embodiment, L101 and L102 are a direct bond.

According to one embodiment of the present specification, Ar101 and Ar102 are the same as or different from each other, and each independently is hydrogen, deuterium, a substituted or unsubstituted alkyl group having 1 to 30 carbon atoms, or a substituted or unsubstituted aryl group having 6 to 60 carbon atoms.

According to another embodiment, Ar101 and Ar102 are the same as or different from each other, and each independently is hydrogen, deuterium, a substituted or unsubstituted alkyl group having 1 to 10 carbon atoms, or a substituted or unsubstituted aryl group having 6 to 30 carbon atoms.

According to another embodiment, Ar101 and Ar102 are the same as or different from each other, and each independently is hydrogen, deuterium, a substituted or unsubstituted phenyl group, or a substituted or unsubstituted naphthyl group.

According to another embodiment, Ar101 and Ar102 are the same as or different from each other, and each independently is hydrogen, deuterium, a phenyl group that is unsubstituted or substituted with deuterium, or a naphthyl group that is unsubstituted or substituted with deuterium.

According to one embodiment of the present specification, R101 to R108 are the same as or different from each other, and each independently is hydrogen, deuterium, a substituted or unsubstituted alkyl group having 1 to 30 carbon atoms, or a substituted or unsubstituted aryl group having 6 to 60 carbon atoms.

According to another embodiment, R101 to R108 are the same as or different from each other, and each independently is hydrogen, deuterium, a substituted or unsubstituted alkyl group having 1 to 10 carbon atoms, or a substituted or unsubstituted aryl group having 6 to 30 carbon atoms.

According to another embodiment, R102 is hydrogen, deuterium, or a substituted or unsubstituted aryl group having 6 to 30 carbon atoms, and R101 and R103 to R108 are the same as or different from each other and each independently is hydrogen or deuterium.

According to another embodiment, R101 to R108 are hydrogen or deuterium.

According to another embodiment, R101 to R108 are all hydrogen.

According to another embodiment, R101 to R108 are all deuterium.

According to one embodiment of the present specification, the compound of Chemical Formula 2 is any one compound selected from among the following compounds:

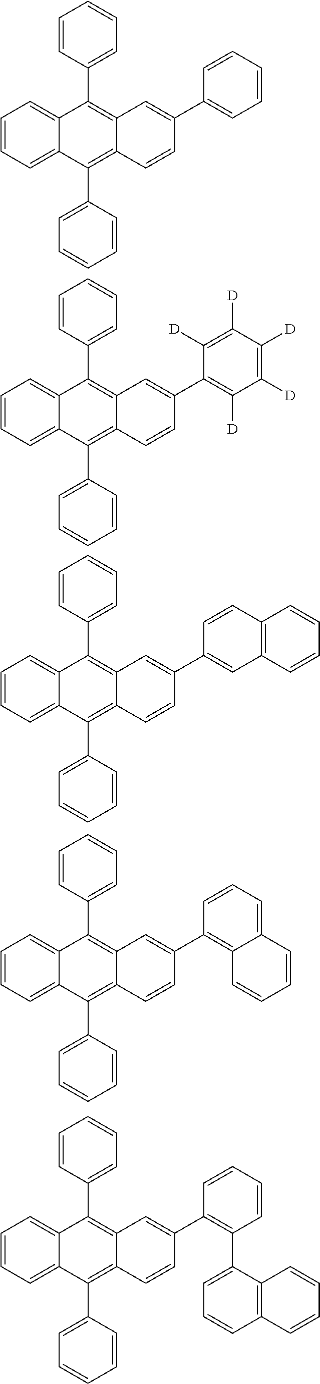

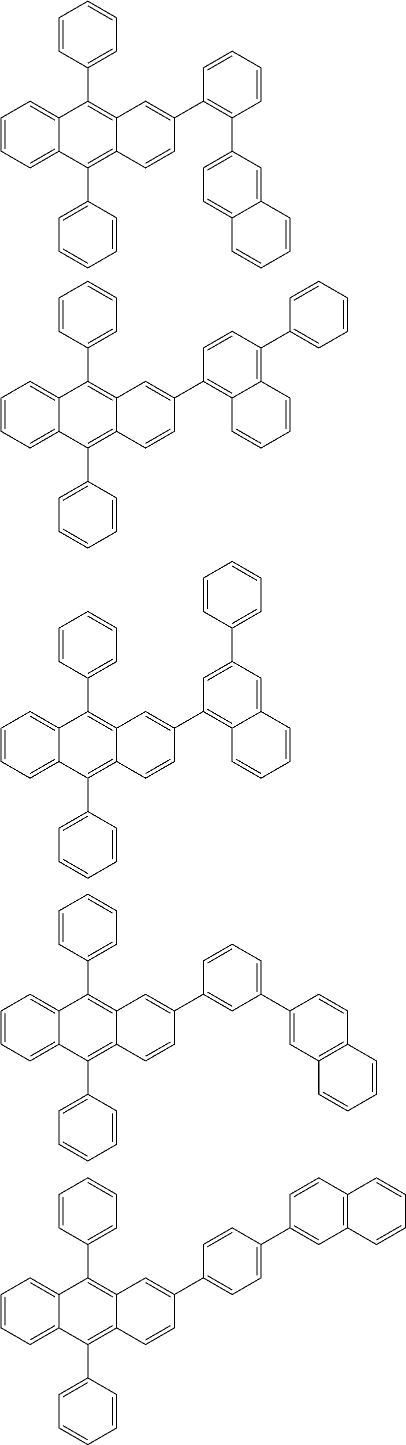

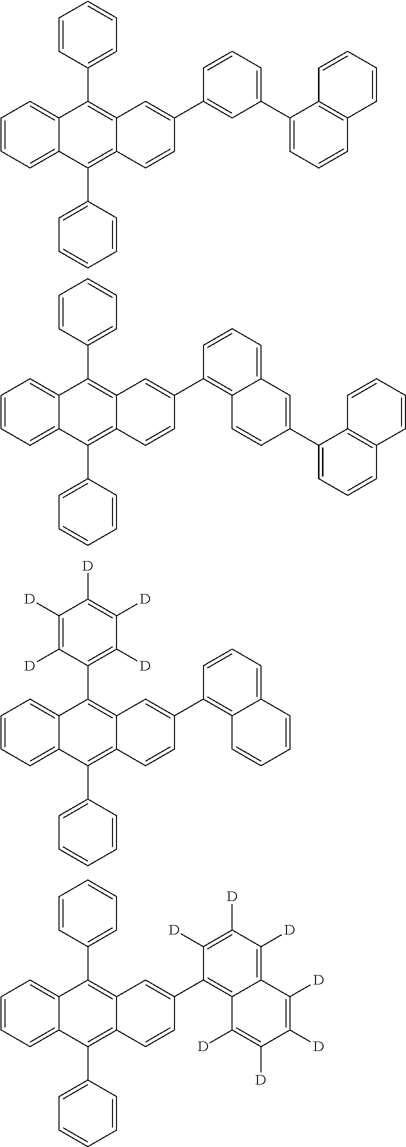

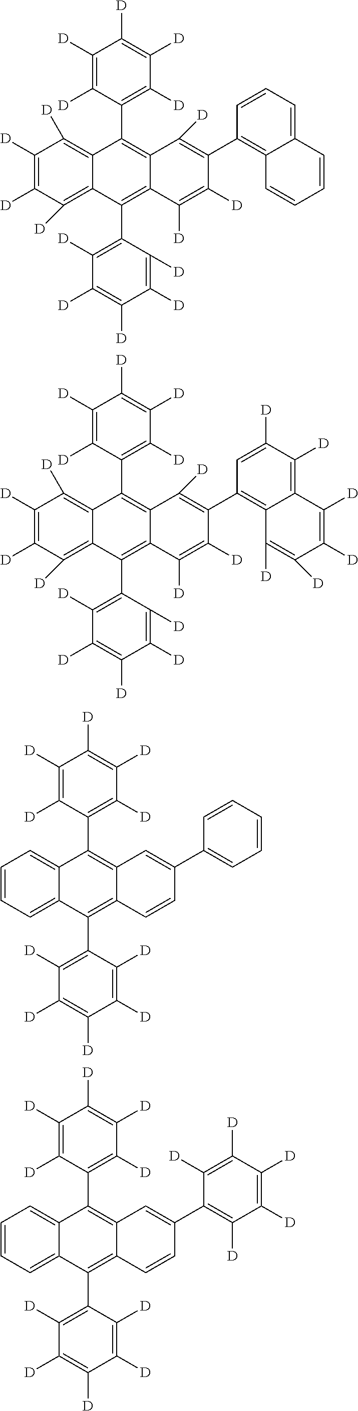

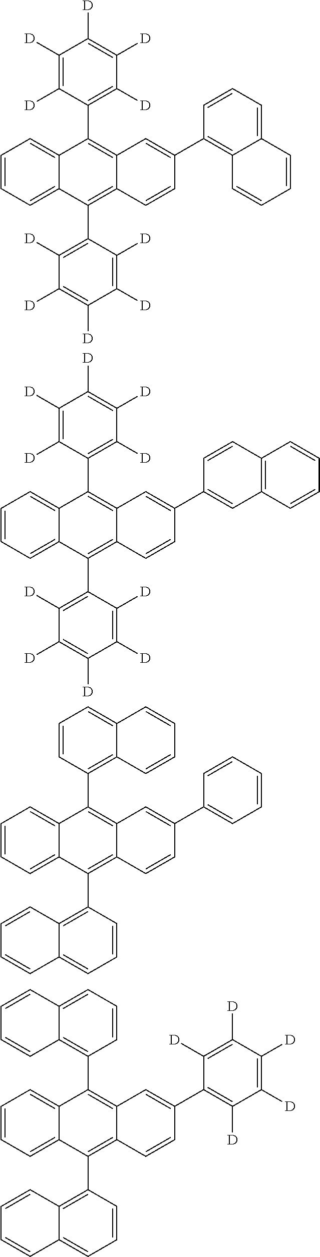

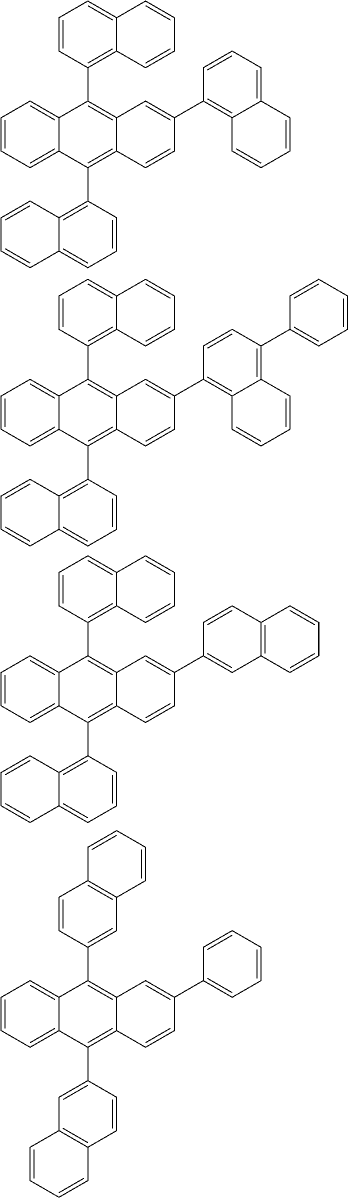

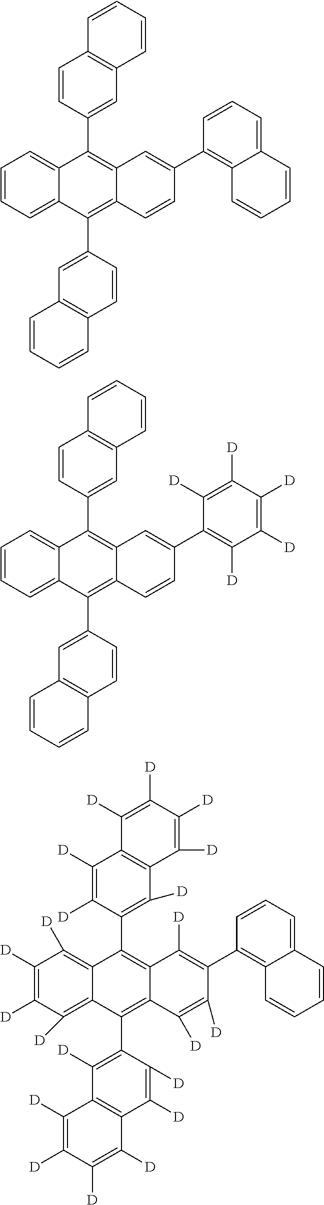

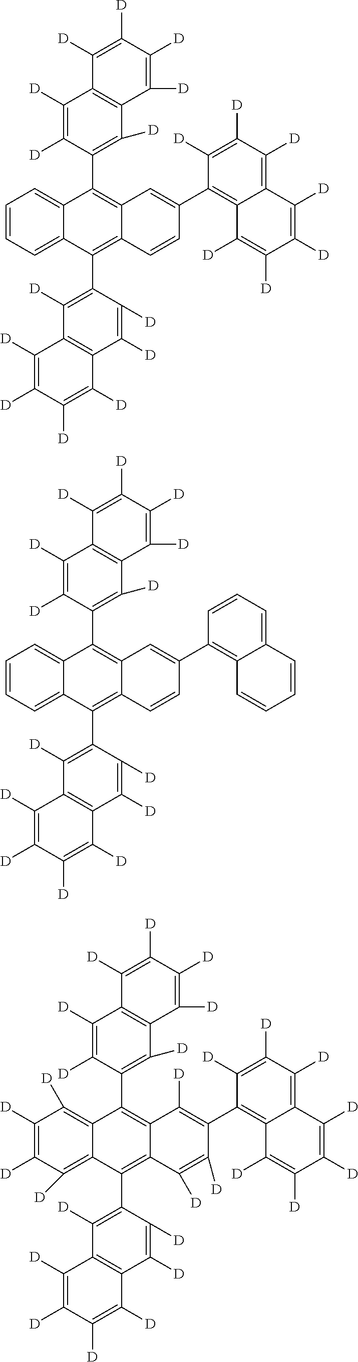

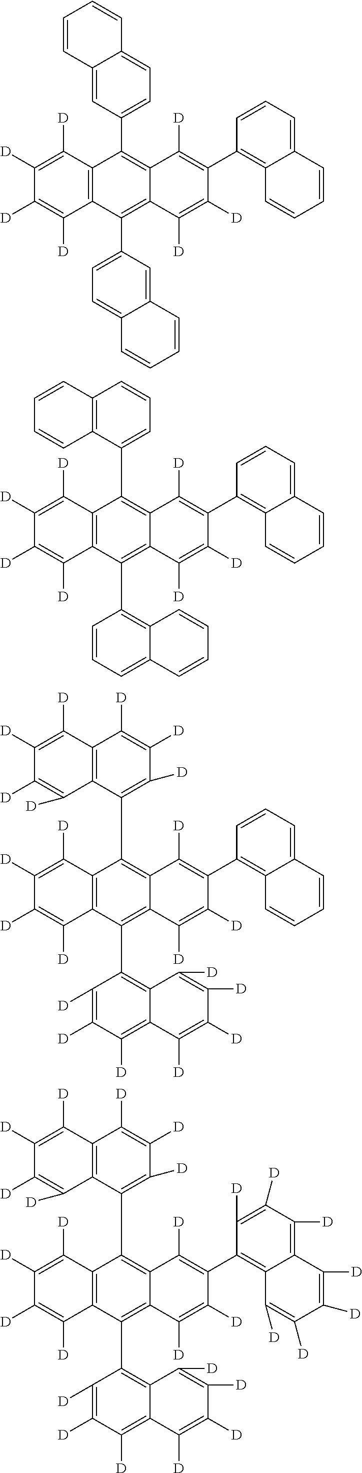

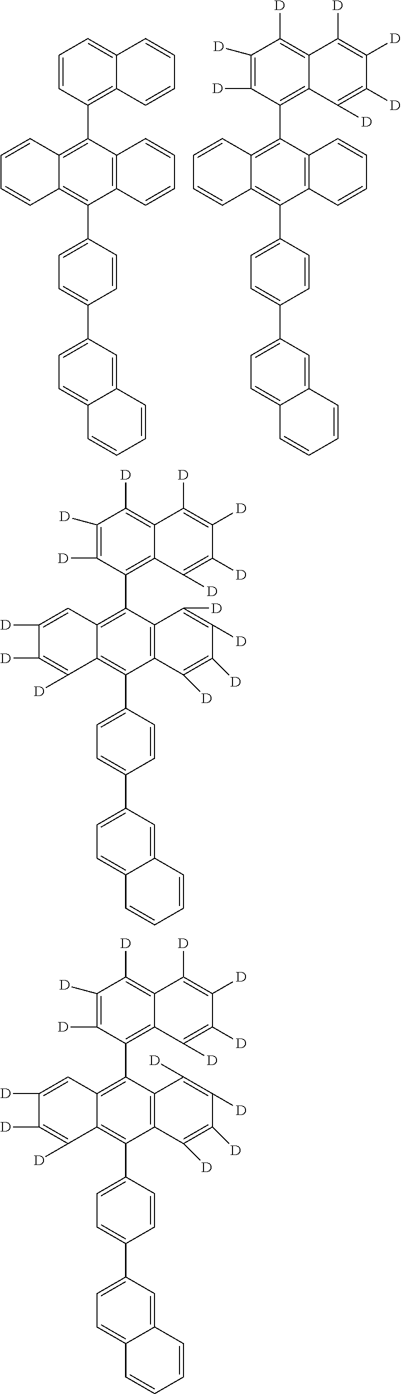

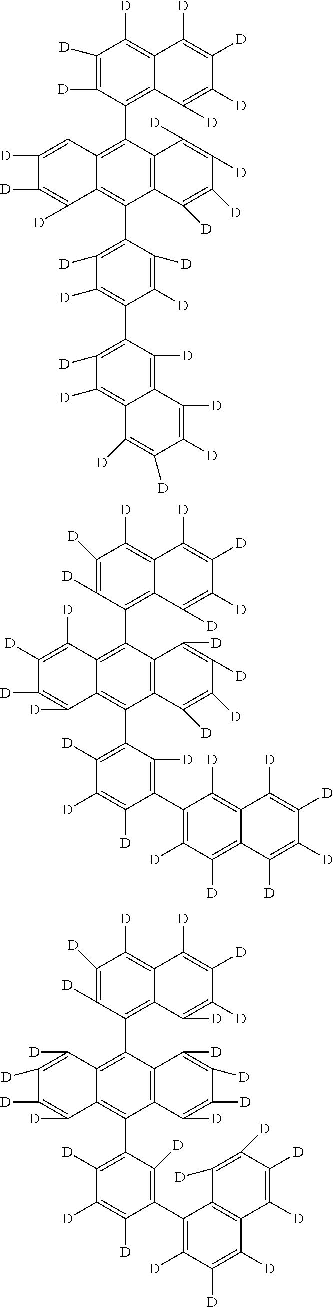

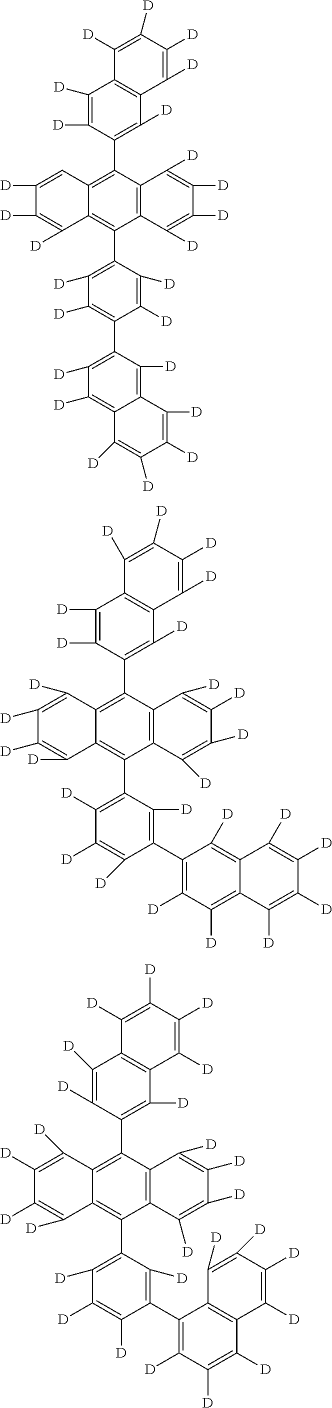

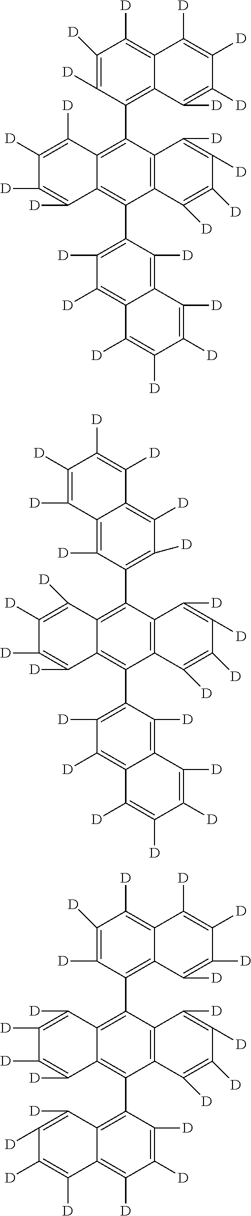

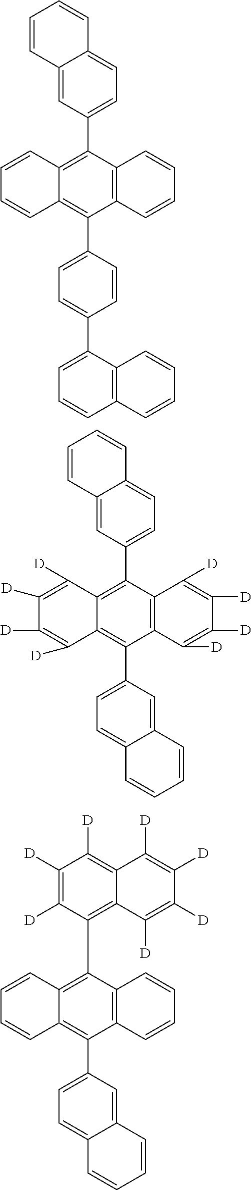

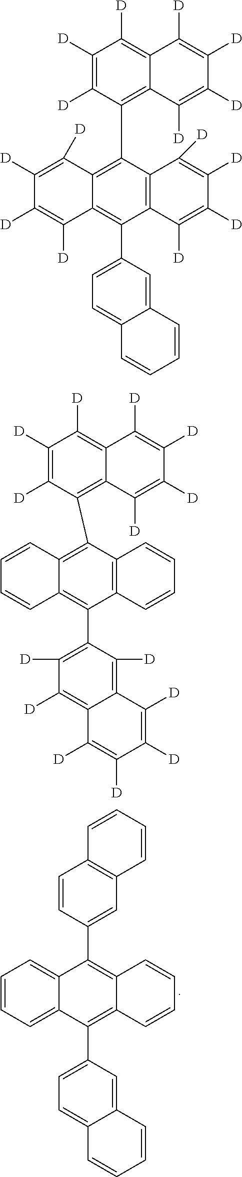

The organic light emitting device of the present specification includes a second organic material layer between a cathode and a light emitting layer, and the second organic material layer includes the compound of Chemical Formula 3.

Hereinafter, Chemical Formula 3 will be described.

According to one embodiment of the present specification, Z is O or S.

According to one embodiment of the present specification, Ar201 and Ar202 are the same as or different from each other, and at least one of Ar201 and Ar202 is -L201-CN, and any remaining is hydrogen.

According to one embodiment of the present specification, any one of Ar201 and Ar202 is -L201-CN, and the remaining one is hydrogen.

According to one embodiment of the present specification, Ar201 is -L201-CN, and Ar202 is hydrogen.

According to one embodiment of the present specification,

Ar202 is -L201-CN, and Ar201 is hydrogen.

According to one embodiment of the present specification, Ar203 and Ar204 are the same as or different from each other, at least one of Ar203 and Ar204 is the following Chemical Formula A-1, and any remaining is hydrogen:

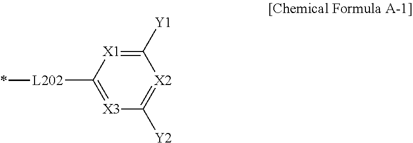

wherein in Chemical Formula A-1:

X1 to X3 are each N or CY3, and at least two of X1 to X3 are N;

Y1 to Y3 are the same as or different from each other, and each independently is hydrogen, deuterium, a substituted or unsubstituted alkyl group, a substituted or unsubstituted hydrocarbon ring group, or a substituted or unsubstituted heterocyclic group;

L202 is a direct bond or a substituted or unsubstituted arylene group; and

* means a bonding position.

According to one embodiment of the present specification, any one of Ar203 and Ar204 is Chemical Formula A-1, and the remaining one is hydrogen.

According to one embodiment of the present specification, Ar203 is Chemical Formula A-1, and Ar204 is hydrogen.

According to one embodiment of the present specification, Ar204 is Chemical Formula A-1, and Ar203 is hydrogen.

According to one embodiment of the present specification, Chemical Formula 3 is any one of the following Chemical Formulae 3-1 to 3-4:

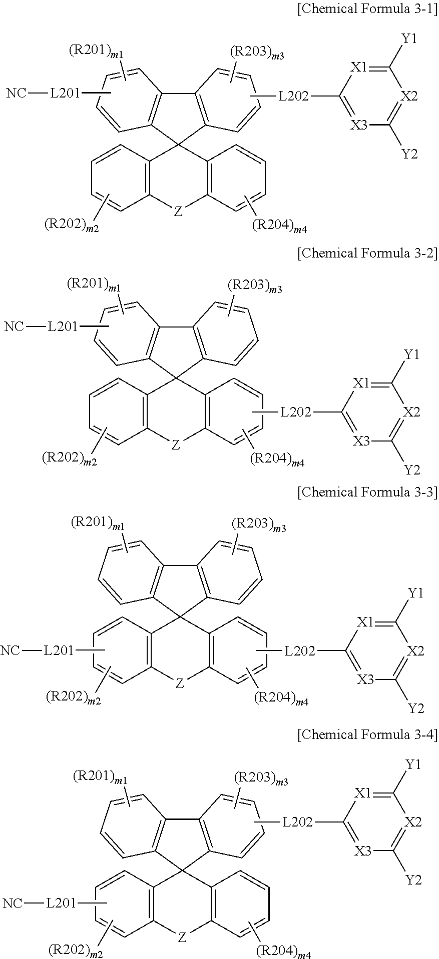

wherein in Chemical Formulae 3-1 to 3-4:

Z, R201 to R204, m1 to m4, L201, L202, X1 to X3, Y1 and Y2 have the same definitions as in Chemical Formula 3.

According to one embodiment of the present specification, L201 is a direct bond or a substituted or unsubstituted arylene group having 6 to 60 carbon atoms.

According to another embodiment, L201 is a direct bond or a substituted or unsubstituted arylene group having 6 to 30 carbon atoms.

According to another embodiment, L201 is a direct bond, a substituted or unsubstituted phenylene group, a substituted or unsubstituted biphenylylene group, or a substituted or unsubstituted naphthylene group.

According to another embodiment, L201 is a direct bond.

According to one embodiment of the present specification, two of X1 to X3 are N, and the remaining one is CY3.

According to one embodiment of the present specification, X1 to X3 are all N.

According to one embodiment of the present specification, Y1 to Y3 are the same as or different from each other, and each independently is hydrogen, deuterium, a substituted or unsubstituted alkyl group having 1 to 30 carbon atoms, a substituted or unsubstituted hydrocarbon ring group having 3 to 60 carbon atoms, or a substituted or unsubstituted heterocyclic group having 2 to 60 carbon atoms.

According to one embodiment of the present specification, Y3 is hydrogen.

According to one embodiment of the present specification, Y1 and Y2 are the same as or different from each other, and each independently is a substituted or unsubstituted phenyl group, a substituted or unsubstituted biphenylene group, or a substituted or unsubstituted naphthyl group.

According to one embodiment of the present specification, L202 is a direct bond or a substituted or unsubstituted arylene group having 6 to 60 carbon atoms.

According to another embodiment, L202 is a direct bond or a substituted or unsubstituted arylene group having 6 to 30 carbon atoms.

According to another embodiment, L202 is a direct bond, a substituted or unsubstituted phenylene group, a substituted or unsubstituted biphenylylene group, or a substituted or unsubstituted naphthylene group.

According to another embodiment, L202 is a substituted or unsubstituted phenylene group or a substituted or unsubstituted biphenylylene group.

According to one embodiment of the present specification, R201 to R204 are the same as or different from each other, and each independently is hydrogen, deuterium, a halogen group, a cyano group, a substituted or unsubstituted alkyl group having 1 to 30 carbon atoms, a substituted or unsubstituted cycloalkyl group having 3 to 30 carbon atoms, a substituted or unsubstituted aryl group having 6 to 60 carbon atoms, or a substituted or unsubstituted heterocyclic group having 2 to 60 carbon atoms.

According to another embodiment, R201 to R204 are the same as or different from each other, and each independently is hydrogen, deuterium, a halogen group, a cyano group, a substituted or unsubstituted alkyl group having 1 to 10 carbon atoms, a substituted or unsubstituted cycloalkyl group having 3 to 20 carbon atoms, a substituted or unsubstituted aryl group having 6 to 30 carbon atoms, or a substituted or unsubstituted heterocyclic group having 2 to 30 carbon atoms.

According to another embodiment, R201 to R204 are the same as or different from each other, and each independently is hydrogen or deuterium.

According to one embodiment of the present specification, m1 is an integer of 0 to 3, and when m1 is 2 or greater, the two or more R201s are the same as or different from each other.

According to one embodiment of the present specification, m2 is an integer of 0 to 3, and when m2 is 2 or greater, the two or more R202s are the same as or different from each other.

According to one embodiment of the present specification, m3 is an integer of 0 to 3, and when m3 is 2 or greater, the two or more R203s are the same as or different from each other.

According to one embodiment of the present specification, m4 is an integer of 0 to 3, and when m4 is 2 or greater, the two or more R204s are the same as or different from each other.

According to one embodiment of the present specification, m1 to m4 are each 0 or 1.

According to one embodiment of the present specification, the compound of Chemical Formula 3 is any one compound selected from among the following compounds:

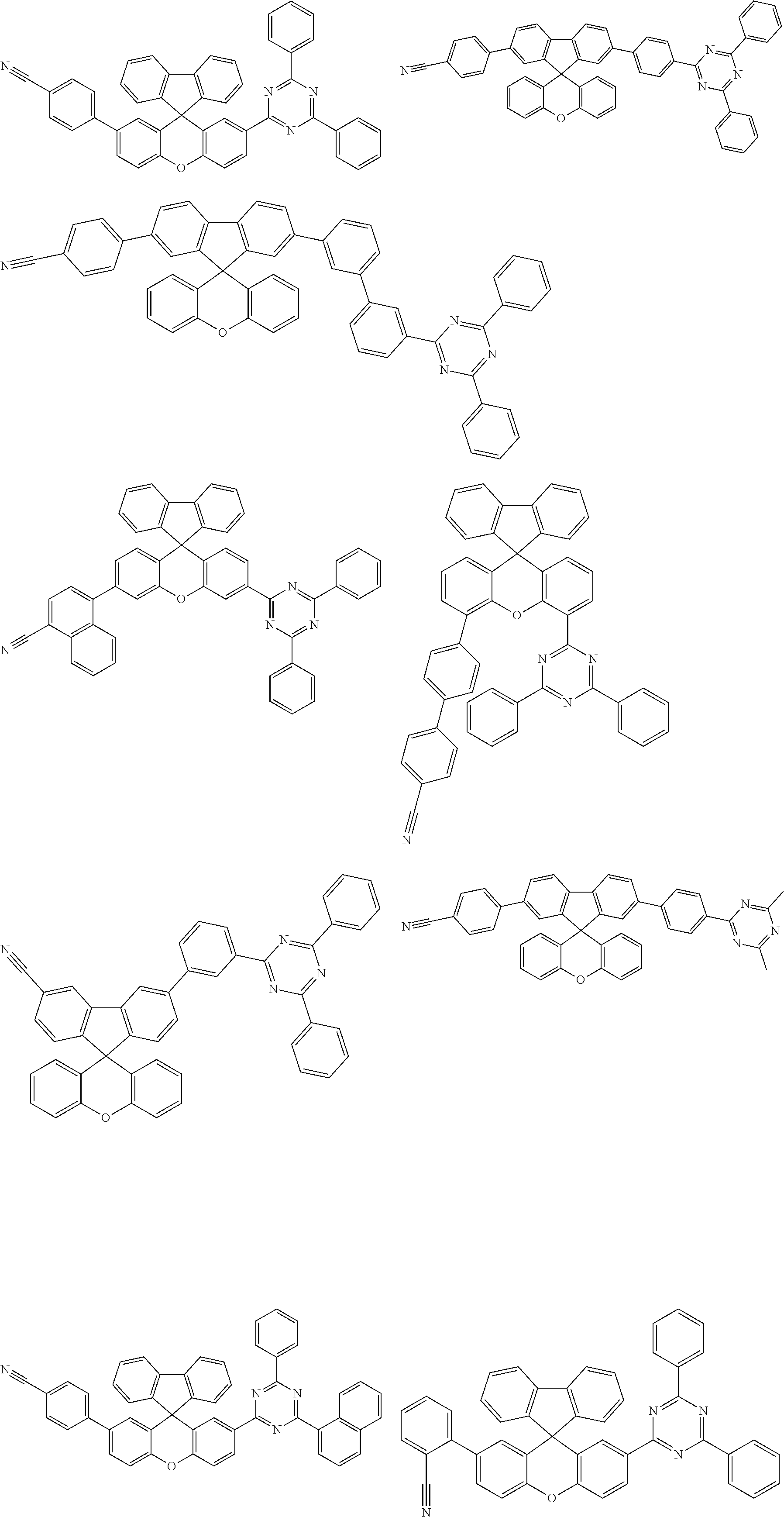

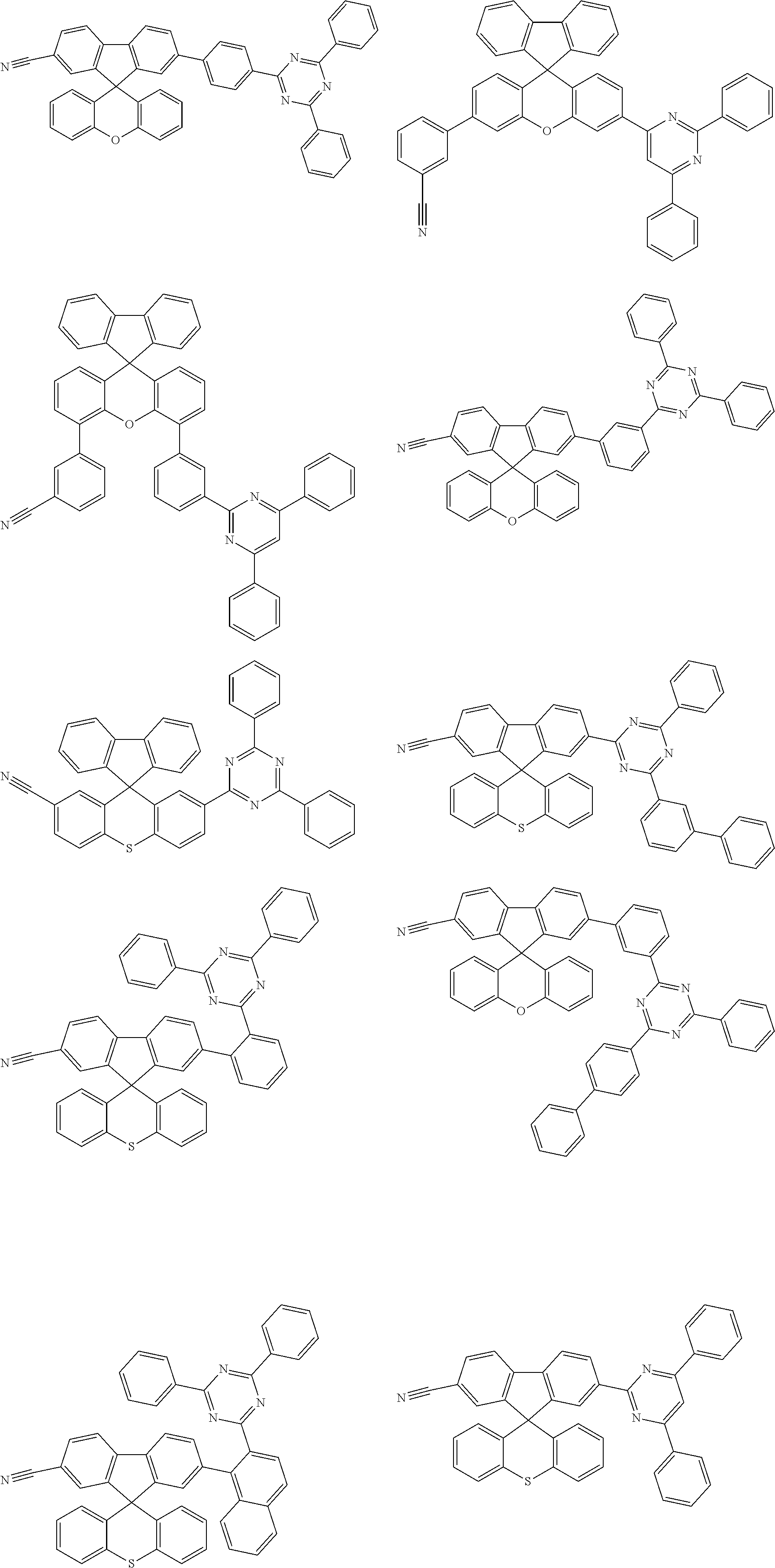

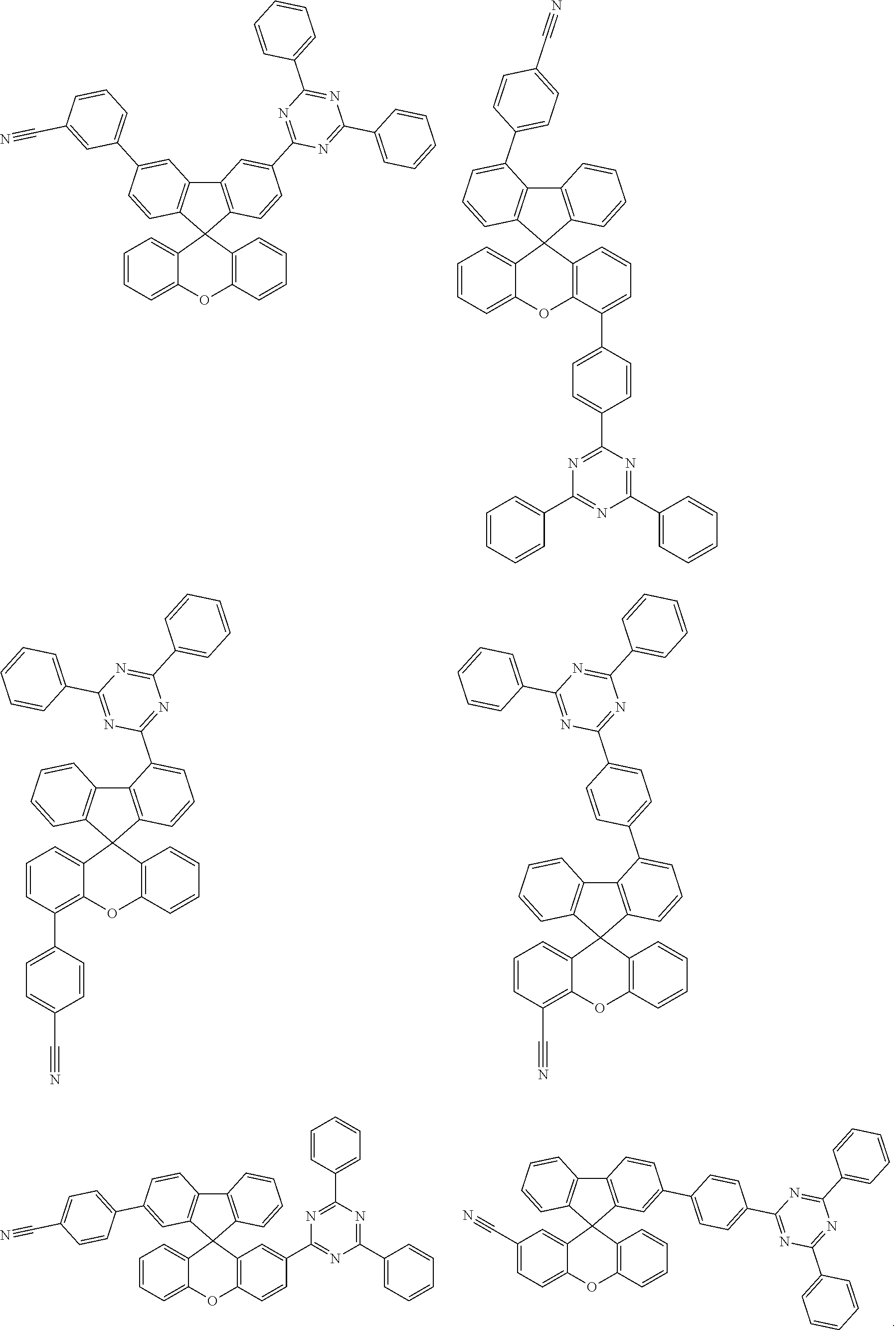

According to one embodiment of the present specification, Chemical Formula 1 and Chemical Formula 3 satisfy at least one of the following Equations 1 and 2:

|E_{H1}|<|E_{H3}| [Equation 1]

|E_{L1}|<|E_{L3}|. [Equation 2]

In Equations 1 and 2,

E_{H1} means a HOMO energy level (eV) of the compound of Chemical Formula 1,

E_{H3} means a HOMO energy level (eV) of the compound of Chemical Formula 3,

E_{L1} means a LUMO energy level (eV) of the compound of Chemical Formula 1, and

E_{L3} means a LUMO energy level (eV) of the compound of Chemical Formula 3.

According to one embodiment of the present specification, Chemical Formula 2 and Chemical Formula 3 satisfy at least one of the following Equations 3 and 4:

E_{S3}>E_{S2} [Equation 3]

E_{T3}>E_{T2} [Equation 4]

In Equations 3 and 4,

E_{S3} means singlet energy (eV) of the compound of Chemical Formula 3,

E_{S2} means singlet energy (eV) of the compound of Chemical Formula 2,

E_{T3} means triplet energy (eV) of the compound of Chemical Formula 3, and

E_{T2} means triplet energy (eV) of the compound of Chemical Formula 2.

In the present specification, the “energy level” means magnitude of energy. Accordingly, the energy level is interpreted to mean an absolute value of the corresponding energy value. For example, the energy level being low or deep means that the absolute value increases in a negative direction from a vacuum level.

In the present specification, a HOMO (highest occupied molecular orbital) means a molecular orbital function where electrons are in an area with highest energy among areas capable of participating in bonding, a LUMO (lowest unoccupied molecular orbital) means a molecular orbital function where electrons are in an area with lowest energy among anti-bonding areas, and a HOMO energy level means a distance from a vacuum level to the HOMO. In addition, a LUMO energy level means a distance from a vacuum level to the LUMO. A determined structure is needed in order to understand electron distribution in a molecule and to understand optical properties. In addition, an electron structure has different structures in neutral, anionic and cationic states depending on the state of charge of the molecule. Although energy levels in neutral, cationic and anionic states are all important in order to drive a device, HOMO (highest occupied molecular orbital) and LUMO (lowest unoccupied molecular orbital) in a neutral state are typically recognized as important properties. In order to determine a molecular structure of a chemical material, a density functional theory is used to optimize an input structure. For the DFT calculation, a BPW91 calculation method (Becke exchange and Perdew correlation-correlation functional) and a DNP (double numerical basis set including polarization functional) basis set are used. The BPW91 calculation method is described in a literature ‘A. D. Becke, Phys. Rev. A, 38, 3098 (1988)’ and ‘J. P. Perdew and Y. Wang, Phys. Rev. B, 45, 13244 (1992)’, and the DNP basis set is described in a literature ‘B. Delley, J. Chem. Phys., 92, 508 (1990)’.

‘DMol3’ package of Biovia can be used to perform calculations using the density functional theory. When an optimal molecular structure is determined using the given method, an energy level that electrons can occupy can be obtained as a result.

In the present specification, the triplet energy means an electronic state with a spin quantum number of 1 in a molecule, and the singlet energy means an electronic state with a spin quantum number of 0. Singlet and triplet energy levels are calculated using a time dependent density functional theory (TD-DFT) in order to obtain properties in an excited state for the optimal molecular structure determined using the above-described method. The density functional calculation can be performed using ‘Gaussian09’ package, a commercial calculation program developed by Gaussian, Inc. A B3PW91 calculation method (Becke exchange and Perdew correlation-correlation functional) and a 6-31G* basis set are used to calculate the time dependent density functional theory. The 6-31G* basis set is described in a literature ‘W. J. Hehre et al., J. Chem. Phys. 56, 2257 (1972)’. Energy obtained when electron arrangements are singlet and triplet for the optimal molecular structure determined using the density functional theory is calculated using a time dependent density functional theory (TD-DFT).

According to one embodiment of the present disclosure, Chemical Formula 1 can be synthesized through an amine substitution reaction, and the reaction is preferably conducted under the presence of a palladium catalyst and a base. The reactive substituent for the amine substitution reaction can be changed depending on information known in the art, and a specific preparation method of Chemical Formula 1 will be described in preparation examples to describe later.

According to one embodiment of the present specification, Chemical Formula 3 can have the core structure prepared as in the following Reaction Formulae 1-1 to 1-4. In the following reaction formulae, substituents can bond using methods known in the art, and types, positions or the number of the substituents can vary depending on technologies known in the art.

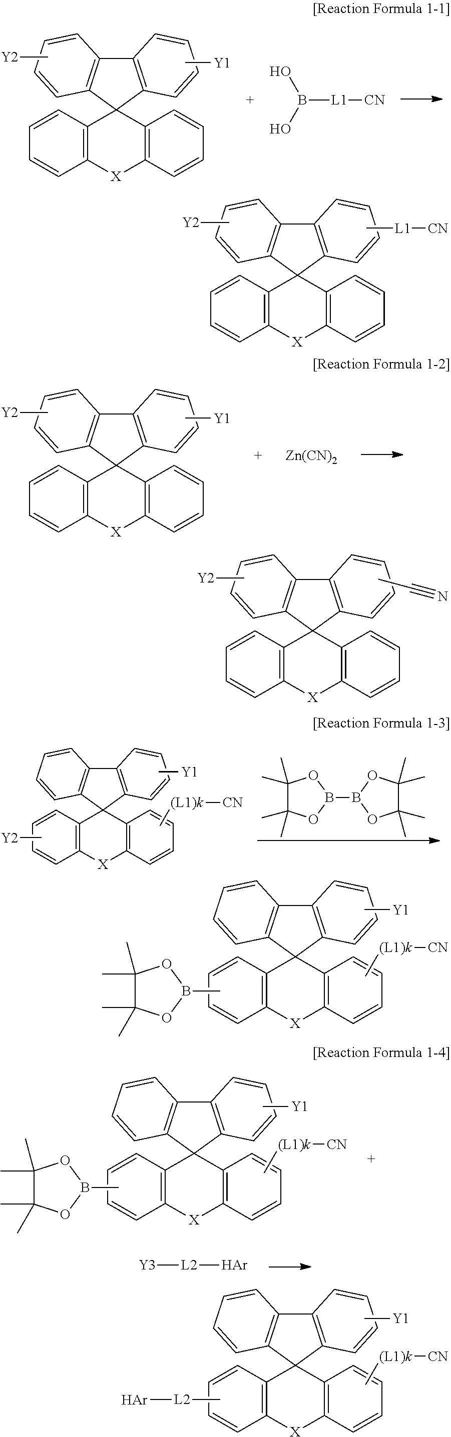

In Reaction Formulae 1-1 to 1-4, L1 means L201 of Chemical Formula 3, L2 means L202 of Chemical Formula 3, HAr means a monocyclic heterocyclic group bonding to L202 in Chemical Formula A-1, and Y1 and Y2 mean a halogen group.

The organic light emitting device of the present specification can be manufactured using common organic light emitting device manufacturing methods and materials except that the first organic material layer is formed using the compound of Chemical Formula 1 described above, the light emitting layer is formed using the compound of Chemical Formula 2 described above, and the second organic material layer is formed using the compound of Chemical Formula 3 described above.

The first organic material layer including the compound of Chemical Formula 1, the light emitting layer including the compound of Chemical Formula 2, and the second organic material layer including the compound of Chemical Formula 3 can be formed into organic material layers using a solution coating method as well as a vacuum deposition method. Herein, the solution coating method means spin coating, dip coating, inkjet printing, screen printing, a spray method, roll coating and the like, but is not limited thereto.

The organic material layer of the organic light emitting device of the present specification can have a structure including the first organic material layer, the light emitting layer and the second organic material layer between the anode and the cathode, however, an additional organic material layer can be further included. For example, the organic light emitting device of the present disclosure can have a structure further including, in addition to the first organic material layer including the compound of Chemical Formula 1, the light emitting layer including the compound of Chemical Formula 2 and the second organic material layer including the compound of Chemical Formula 3, a hole injection layer, a hole transfer layer, a layer carrying out hole transfer and hole injection at the same time, an electron blocking layer, a light emitting layer, an electron transfer layer, an electron injection layer, a layer carrying out electron transfer and electron injection at the same time, a hole blocking layer and the like as the additional organic material layer. However, the structure of the organic light emitting device is not limited thereto, and can include a smaller number or a larger number of organic material layers.

According to one embodiment of the present specification, one or more organic material layers are further included between the anode and the first organic material layer. The one or more organic material layers can each be a hole injection layer or a hole transfer layer.

In one embodiment of the present specification, two organic material layers are further included between the anode and the first organic material layer.

According to one embodiment of the present specification, the first organic material layer is adjacent to the light emitting layer.

According to one embodiment of the present specification, the first organic material layer is a hole injection layer, a hole transfer layer or an electron blocking layer.

According to one embodiment of the present specification, the first organic material layer is an electron blocking layer.

According to one embodiment of the present specification, the first organic material layer is an electron blocking layer, and one or more of a hole injection layer and a hole transfer layer can be further fainted between the anode and the first organic material layer.

According to one embodiment of the present specification, one or more organic material layers are further included between the light emitting layer and the second organic material layer. The one or more organic material layers can each be an electron transfer layer or a hole blocking layer.

According to one embodiment of the present specification, one organic material layer is further included between the light emitting layer and the second organic material layer.

According to one embodiment of the present specification, one or more organic material layers are further included between the cathode and the second organic material layer.

According to one embodiment of the present specification, the second organic material layer is an electron transfer layer or a hole blocking layer.

According to one embodiment of the present specification, the second organic material layer includes the compound of Chemical Formula 3 described above, and can include an additional compound or an additional metal material. Examples of the metal material can include LiQ. When the second organic material layer includes an additional compound or metal material together with the compound of Chemical Formula 3, the compound of Chemical Formula 3 to the additional compound or metal material have a mass ratio of approximately 3:7 to 7:3.

According to one embodiment of the present specification, the second organic material layer is an electron transfer layer.

According to one embodiment of the present specification, the second organic material layer is an electron transfer layer, and a hole blocking layer can be further foamed between the cathode and the second organic material layer.

According to one embodiment of the present specification, the light emitting layer includes the compound of Chemical Formula 2 described above, and further includes a dopant.

According to another embodiment, the light emitting layer includes the compound of Chemical Formula 2 described above as a host of the light emitting layer, and further includes a dopant. Examples of the dopant can include pyrene-based compounds, but are not limited thereto.

In the organic light emitting device according to one embodiment of the present specification, the dopant can be included in 0.1 parts by weight to 50 parts by weight in the light emitting layer based on 100 parts by weight of the host. According to another embodiment, the dopant can be included in 1 parts by weight to 30 parts by weight in the light emitting layer based on 100 parts by weight of the host. Energy is efficiently transferred from the host to the dopant when the dopant content is in the above-mentioned range.

According to one embodiment of the present specification, the light emitting layer including the compound of Chemical Formula 2 emits blue light.

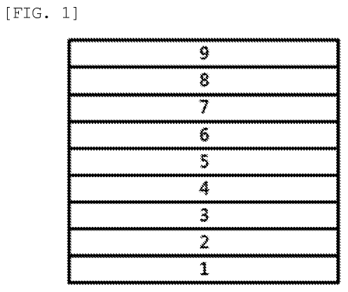

The organic light emitting device of the present disclosure can have a structure as illustrated in FIG. 1, however, the structure is not limited thereto.

FIG. 1 illustrates a structure of the organic light emitting device in which an anode (2), a hole injection layer (3), a hole transfer layer (4), an electron blocking layer (5), a light emitting layer (6), a hole blocking layer (7), an electron transfer layer (8) and a cathode (9) are consecutively laminated on a substrate (1). The light emitting layer (6) can include the compound of Chemical Formula 2 described above, the electron blocking layer (5) can include the compound of Chemical Formula 1 described above, and the electron transfer layer (8) can include the compound of Chemical Formula 3 described above.

For example, the organic light emitting device of the present specification can be manufactured by forming an anode on a substrate by depositing a metal, a metal oxide having conductivity, or an alloy thereof using a physical vapor deposition (PVD) method such as sputtering or e-beam evaporation, and foaming an organic material layer including a hole injection layer, a hole transfer layer, a light emitting layer, an electron blocking layer, a hole blocking layer, an electron transfer layer, an electron injection layer and the like thereon, and then depositing a material usable as a cathode thereon. In addition to such a method, the organic light emitting device can also be manufactured by consecutively depositing a cathode material, an organic material layer and an anode material on a substrate.

The anode is an electrode that injects holes, and as the anode material, materials having large work function are normally preferred so that hole injection to an organic material layer is smooth. Specific examples of the anode material usable in the present disclosure include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene] (PEDOT), polypyrrole and polyaniline, but are not limited thereto.

The cathode is an electrode that injects electrons, and as the cathode material, materials having small work function are normally preferred so that electron injection to an organic material layer is smooth. Specific examples of the cathode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

The hole injection layer is a layer performing a role of smoothly injecting holes from an anode to a light emitting layer. The hole injection material is a material capable of favorably receiving holes from an anode at a low voltage, and the highest occupied molecular orbital (HOMO) of the hole injection material is preferably in between the work function of the anode material and the HOMO of surrounding organic material layers. Specific examples of the hole injection material include metal porphyrins, oligothiophene, arylamine-based organic materials, hexanitrile hexaazatriphenylene-based organic materials, quinacridone-based organic materials, perylene-based organic materials, anthraquinone, and polyaniline- and polythiophene-based conductive polymers, and the like, but are not limited thereto.

The hole transfer layer can perform a role of smoothly transferring holes. As the hole transfer material, materials capable of receiving holes from an anode or a hole injection layer, moving the holes to a light emitting layer, and having high mobility for the holes are suited. Specific examples thereof include arylamine-based organic materials, conductive polymers, block copolymers having conjugated parts and non-conjugated parts together, and the like, but are not limited thereto.

An electron blocking layer can be provided between the hole transfer layer and the light emitting layer. As the electron blocking layer, the compound of Chemical Formula 1 described above, or materials known in the art can be used.

The organic light emitting device of the present disclosure can include an additional light emitting layer in addition to the light emitting layer including the compound of Chemical Formula 2. Herein, the light emitting layer can emit red, green or blue light, and can be formed with a phosphorescent material or a fluorescent material. The light emitting material is a material capable of emitting light in a visible region by receiving holes and electrons from a hole transfer layer and an electron transfer layer, respectively, and binding the holes and the electrons, and is preferably a material having favorable quantum efficiency for fluorescence or phosphorescence.

As the host material of the additional light emitting layer, fused aromatic ring derivatives, heteroring-containing compounds or the like can be included. Specifically, anthracene derivatives, pyrene derivatives, naphthalene derivatives, pentacene derivatives, phenanthrene compounds, fluoranthene compounds or the like can be included as the fused aromatic ring derivative, and carbazole derivatives, dibenzofuran derivatives, ladder-type furan compounds, pyrimidine derivatives or the like can be included as the heteroring-containing compound, however, the host material is not limited thereto.

As the light emitting dopant of the additional light emitting layer, phosphorescent materials such as bis(1-phenylisoquinoline) acetylacetonate iridium (PIQIr(acac)), bis(1-phenylquinoline)acetylacetonate iridium (PQIr(acac)), tris(1-phenylquinoline)iridium (PQIr) or octaethylporphyrin platinum (PtOEP), or fluorescent materials such as tris(8-hydroxyquinolino)aluminum (Alq₃) can be used as the light emitting dopant, however, the light emitting dopant is not limited thereto. When the light emitting layer emits green light, phosphorescent materials such as fac tris(2-phenylpyridine)iridium (Ir(ppy)₃), or fluorescent materials such as tris(8-hydroxyquinolino)aluminum (Alq₃) can be used as the light emitting dopant, however, the light emitting dopant is not limited thereto. When the light emitting layer emits blue light, phosphorescent materials such as (4,6-F2ppy)₂Irpic, or fluorescent materials such as spiro-DPVBi, spiro-6P, distyrylbenzene (DSB), distyrylarylene (DSA), PFO-based polymers or PPV-based polymers can be used as the light emitting dopant, however, the light emitting dopant is not limited thereto.

In one embodiment of the present specification, a hole blocking layer can be provided between the electron transfer layer and the light emitting layer, and materials known in the art can be used as the hole blocking layer.

The electron transfer layer can perform a role of smoothly transferring electrons. As the electron transfer material, materials capable of favorably receiving electrons from a cathode, moving the electrons to a light emitting layer, and having high mobility for the electrons are suited. Specific examples thereof include the compound of Chemical Formula 3 described above, Al complexes of 8-hydroxyquinoline; complexes including Alq₃; organic radical compounds; hydroxyflavon-metal complexes, and the like, but are not limited thereto.

The electron injection layer can perform a role of smoothly injecting electrons. As the electron injection material, compounds having an electron transferring ability, having an electron injection effect from a cathode, having an excellent electron injection effect for a light emitting layer or light emitting material, and preventing excitons generated in the light emitting layer from moving to a hole injection layer, and in addition thereto, having an excellent thin film forming ability are preferred. Specific examples thereof can include fluorenone, anthraquinodimethane, diphenoquinone, thiopyran dioxide, oxazole, oxadiazole, triazole, imidazole, perylene tetracarboxylic acid, fluorenylidene methane, anthrone or the like, and derivatives thereof, metal complex compounds, nitrogen-containing 5-membered ring derivatives, and the like, but are not limited thereto.

The metal complex compound includes 8-hydroxyquinolinato lithium, bis(8-hydroxyquinolinato)zinc, bis(8-hydroxy-quinolinato)copper, bis(8-hydroxyquinolinato)-manganese, tris(8-hydroxyquinolinato)aluminum, tris(2-methyl-8-hydroxy-quinolinato)aluminum, tris(8-hydroxyquinolinato)-gallium, bis(10-hydroxybenzo[h]quinolinato)beryllium, bis(10-hydroxy-benzo[h]quinolinato)zinc, bis(2-methyl-8-quinolinato)-chlorogallium, bis(2-methyl-8-quinolinato)(o-cresolato)-gallium, bis(2-methyl-8-quinolinato)(1-naphtholato)aluminum, bis(2-methyl-8-quinolinato)(2-naphtholato)gallium and the like, but is not limited thereto.

The organic light emitting device according to the present disclosure can be a top-emission type, a bottom-emission type or a dual-emission type depending on the materials used.

### EXAMPLES

Hereinafter, the present specification will be described in more detail with reference to examples. However, the following examples are for illustrative purposes only, and not for limiting the present specification.

### Preparation Example

### Preparation Example 1

### Preparation of Compound EBL-1

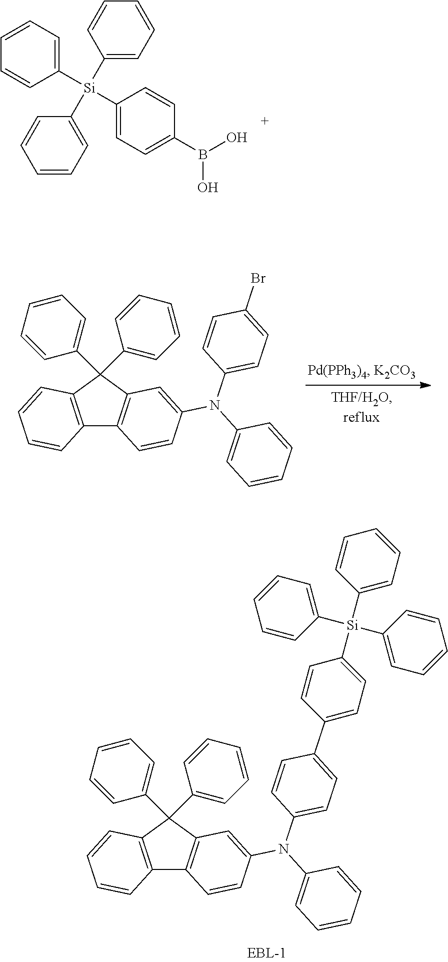 [SMILES: OB(O)c1ccc([Si](c2ccccc2)(c2ccccc2)c2ccccc2)cc1 ; Brc1ccc(N(c2ccccc2)c2ccc3c(c2)C(c2ccccc2)(c2ccccc2)c2ccccc2-3)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccccc2)c2ccccc2-3)cc1]

After completely dissolving (4-(triphenylsilyl)-phenyl)boronic acid (6.34 g, 16.68 mmol) and N-(4-bromophenyl)-N,9,9-triphenyl-9H-fluoren-2-amine (8.57 g, 15.17 mmol) in tetrahydrofuran (240 ml) in a 500 ml round bottom flask under a nitrogen atmosphere, a 2 M aqueous potassium carbonate solution (120 ml) was introduced thereto and tetrakis-(triphenylphosphine)palladium (0.53 g, 0.46 mmol) was introduced thereto, and then the result was stirred for 3 hours while heating. After lowering the temperature to room temperature, the water layer was removed, and the result was dried with anhydrous magnesium sulfate, then vacuum concentrated, and recrystallized with ethyl acetate (240 ml) to prepare Compound EBL-1 (8.89 g, 71%).

MS[M+H]⁺=821

### Preparation Example 2

### Preparation of Compound EBL-2

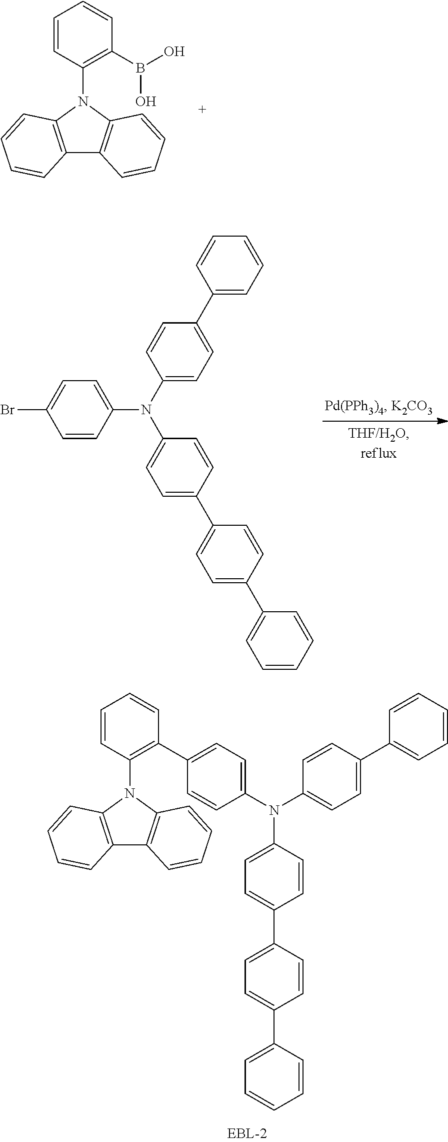 [SMILES: OB(O)c1ccccc1-n1c2ccccc2c2ccccc21 ; Brc1ccc(N(c2ccc(-c3ccccc3)cc2)c2ccc(-c3ccc(-c4ccccc4)cc3)cc2)cc1 ; c1ccc(-c2ccc(-c3ccc(N(c4ccc(-c5ccccc5)cc4)c4ccc(-c5ccccc5-n5c6ccccc6c6ccccc65)cc4)cc3)cc2)cc1]

After completely dissolving N-([1,1′-biphenyl]-4-yl)-N-(4-bromophenyl)-[1,1′:4′,1″-terphenyl]-4-amine (6.74 g, 12.23 mmol) and (2-(9H-carbazol-9-yl)phenyl)boronic acid (4.04 g, 14.07 mmol) in tetrahydrofuran (240 ml) in a 500 ml round bottom flask under a nitrogen atmosphere, a 2 M aqueous potassium carbonate solution (120 ml) was introduced thereto and tetrakis-(triphenylphosphine)palladium (0.42 g, 0.37 mmol) was introduced thereto, and then the result was stirred for 3 hours while heating. After lowering the temperature to room temperature, the water layer was removed, and the result was dried with anhydrous magnesium sulfate, then vacuum concentrated, and recrystallized with ethyl acetate (240 ml) to prepare Compound EBL-2 (6.11 g, 70%).

MS[M+H]⁺=715

### Preparation Example 3

### Preparation of Compound EBL-3

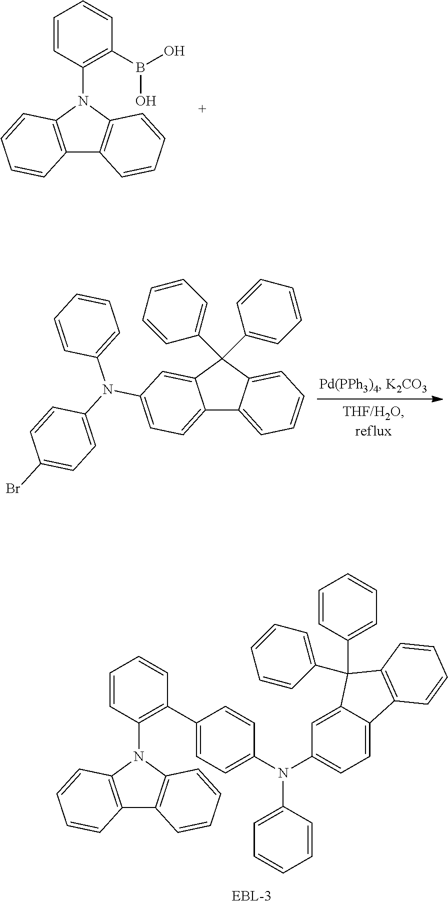 [SMILES: OB(O)c1ccccc1-n1c2ccccc2c2ccccc21 ; Brc1ccc(N(c2ccccc2)c2ccc3c(c2)C(c2ccccc2)(c2ccccc2)c2ccccc2-3)cc1 ; c1ccc(N(c2ccc(-c3ccccc3-n3c4ccccc4c4ccccc43)cc2)c2ccc3c(c2)C(c2ccccc2)(c2ccccc2)c2ccccc2-3)cc1]

After completely dissolving N-(4-bromophenyl)-N,9,9-triphenyl-9H-fluoren-2-amine (8.45 g, 22.06 mmol) and (2-(9H-carbazol-9-yl)phenyl)boronic acid (5.06 g, 17.64 mmol) in tetrahydrofuran (240 ml) in a 500 ml round bottom flask under a nitrogen atmosphere, a 2 M aqueous potassium carbonate solution (120 ml) was introduced thereto and tetrakis-(triphenylphosphine)palladium (0.53 g, 0.46 mmol) was introduced thereto, and then the result was stirred for 3 hours while heating. After lowering the temperature to room temperature, the water layer was removed, and the result was dried with anhydrous magnesium sulfate, then vacuum concentrated, and recrystallized with ethyl acetate (250 ml) to prepare Compound EBL-3 (7.16 g, 63%).

MS[M+H]⁺=727

### Preparation Example 4

### Preparation of Compound HOST-2

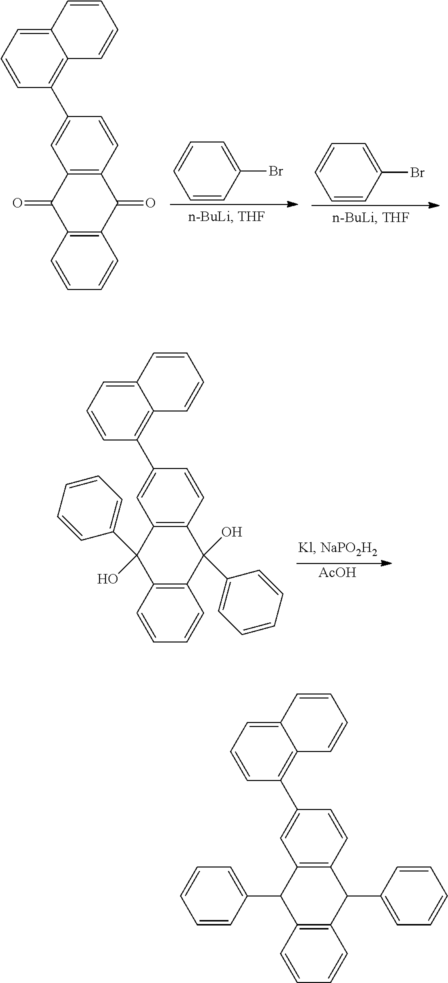

After dissolving phenyl bromide (1 eq.) in tetrahydrofuran (THF) under a nitrogen atmosphere, n-BuLi (1.1 eq.) was slowly added dropwise thereto at −78° C. After 30 minutes, 2-(naphthalen-1-yl)anthracene-9,10-dione (1 eq.) was added thereto. The temperature was raised to room temperature, and when the reaction was finished, the result was extracted with ethyl acetate and then washed with water. Such a method was performed once more using phenyl bromide. After the reaction was finished, the result was extracted with ethyl acetate and then washed with water. After evaporating all the ethyl acetate, solids were dropped using hexane to obtain 2-(naphthalen-1-yl)-9,10-diphenyl-9,10-dihydroanthracene-9,10-diol in a 50% yield. The 2-(Naphthalen-1-yl)-9,10-diphenyl-9,10-dihydroanthracene-9,10-diol (1 eq.), KI (3 eq.) and NaPO₂H₂ (5 eq.) were introduced to acetic acid, and the result was refluxed after raising the temperature. After the reaction was finished, an excess amount of water was poured thereinto, and produced solids were filtered. The result was extracted with ethyl acetate, then washed with water, and recrystallized with toluene to obtain HOST-2 in a 70% yield. [cal. m/s: 456.19, exp. m/s (M+) 456.5]

### Preparation Example 5

### Preparation of Compound HOST-3

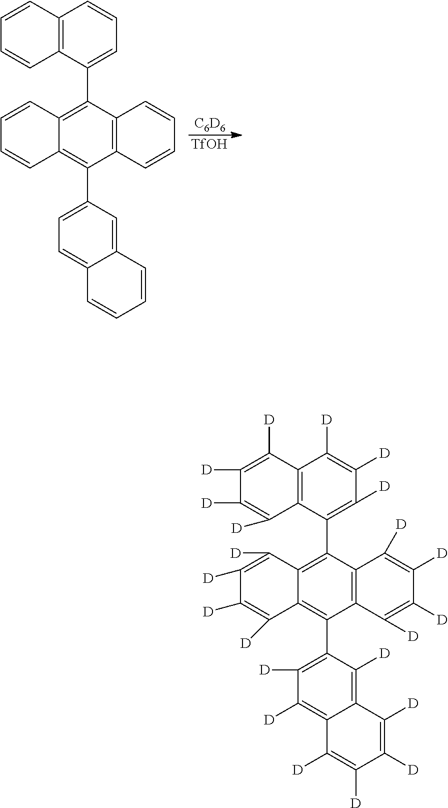

9-(Naphthalen-1-yl)-10-(naphthalen-2-yl)anthracene (20 g) and trifluoromethanesulfonic acid (2 g, 0.1 v) were introduced to C6D6 (500 ml, 25 v), and the mixture was stirred for 2 hours at 70° C. After the reaction was finished, D₂O (60 ml) was introduced thereto, and after stirring the result for 30 minutes, trimethylamine (6 ml) was added dropwise thereto. The reaction solution was transferred to a separatory funnel, and extracted with water and toluene. The extract was dried with MgSO₄, and then recrystallized with ethyl acetate to obtain HOST-3 in a 64% yield. [cal. m/s: 430.55, exp. m/s (M+) 448 to 452 (molecular weight appears as distribution due to nature of deuterium substitution)]

### Preparation Example 6

### Preparation of Compound ETL-1

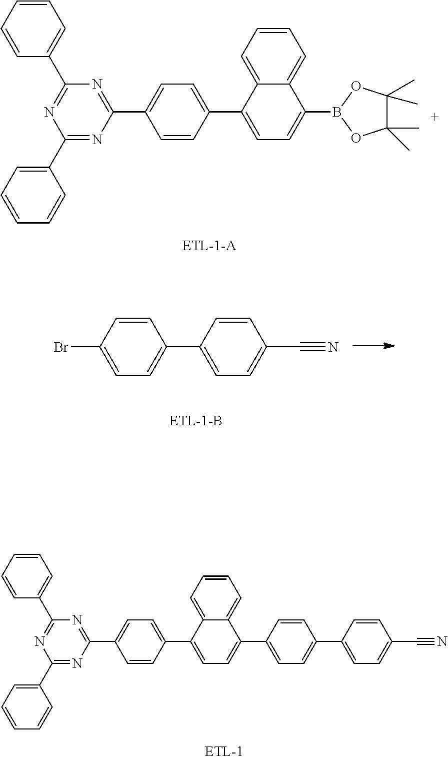 [SMILES: CC1(C)OB(c2ccc(-c3ccc(-c4nc(-c5ccccc5)nc(-c5ccccc5)n4)cc3)c3ccccc23)OC1(C)C ; N#Cc1ccc(-c2ccc(Br)cc2)cc1 ; N#Cc1ccc(-c2ccc(-c3ccc(-c4ccc(-c5nc(-c6ccccc6)nc(-c6ccccc6)n5)cc4)c4ccccc34)cc2)cc1]

ETL-1-A (12 g, 21.4 mmol) and ETL-1-B (5.5 g, 21.4 mmol) were introduced to tetrahydrofuran (240 ml) under a nitrogen atmosphere, and the mixture was stirred and refluxed. After that, potassium carbonate (8.9 g, 64.1 mmol) dissolved in water (9 ml) was introduced thereto, and, after sufficiently stirring the result, tetrakistriphenyl-phosphinopalladium (0.7 g, 0.6 mmol) was introduced thereto. After reacting for 3 hours, the result was cooled to room temperature, then separated into an organic layer and a water layer, and the organic layer was distilled. This was introduced to chloroform (20 times, 262 mL) again and dissolved therein, and washed twice with water. The organic layer was separated, stirred after introducing anhydrous magnesium sulfate thereto, then filtered, and the filtrate was vacuum distilled. The concentrated compound was recrystallized with chloroform and ethyl acetate to prepare white solid Compound ETL-1 (9.8 g, yield 75%).

MS [M+H]⁺=613

### Preparation Example 7

### Preparation of Compound ETL-2

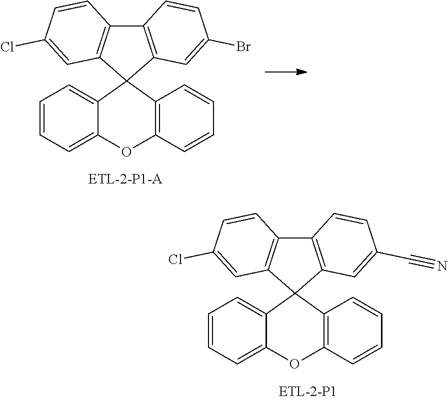 [SMILES: Clc1ccc2c(c1)C1(c3ccccc3Oc3ccccc31)c1cc(Br)ccc1-2 ; N#Cc1ccc2c(c1)C1(c3ccccc3Oc3ccccc31)c1cc(Cl)ccc1-2]

After completely dissolving the compound of Chemical Formula ETL-2-P1-A (20 g, 44.9 mmol) and zinc cyanide compound (2.6 g, 22.4 mmol) in dimethylacetamide (200 mL), tetrakistriphenyl-phosphinopalladium (1.6 g, 1.34 mmol) was introduced thereto, and the result was stirred for 2 hours while heating. After lowering the temperature to room temperature and terminating the reaction, water (200 ml) was introduced thereto to filter white solids. The filtered white solids were washed twice each with ethanol and water to prepare the compound of Chemical Formula ETL-2-P1 (14.1 g, yield 80%).

MS [M+H]⁺=392

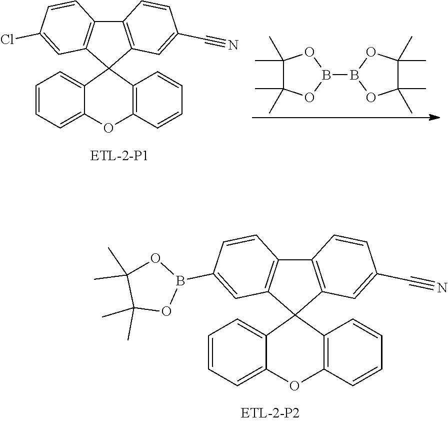

ETL-2-P1 (20 g, 51 mmol) and bis(pinacolato)diboron (20.2 g, 51 mmol) were introduced to Diox (400 ml) under a nitrogen atmosphere, and the mixture was stirred and refluxed. After that, potassium phosphate tribasic (32.5 g, 153.1 mmol) was introduced thereto, and, after sufficiently stirring the result, dibenzylideneacetone palladium (0.9 g, 1.5 mmol) and tricyclohexylphosphine (0.9 g, 3.1 mmol) were introduced thereto. After reacting for 6 hours, the result was cooled to room temperature, the organic layer was filtered to remove the salt, and the filtered organic layer was distilled. This was introduced to chloroform (10 times, 247 mL) again and dissolved therein, and washed twice with water. The organic layer was separated, stirred after introducing anhydrous magnesium sulfate thereto, then filtered, and the filtrate was vacuum distilled. The concentrated compound was recrystallized with chloroform and ethanol to prepare white solid Compound ETL-2-P2 (12.6 g, yield 51%).

MS [M+H]⁺=484

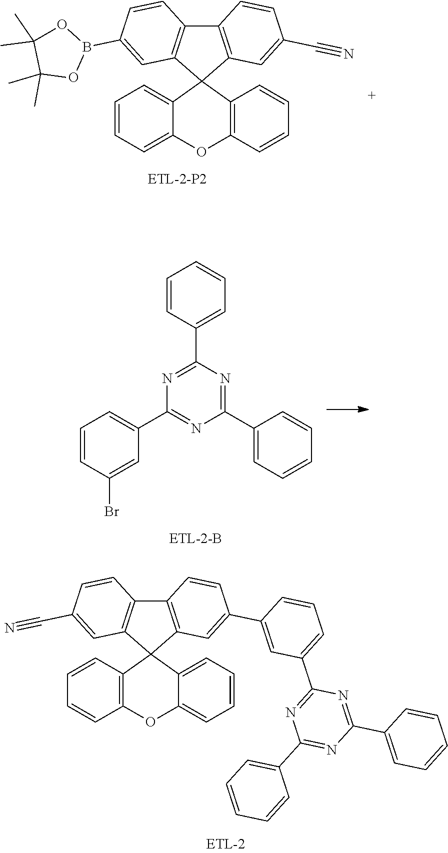 [SMILES: CC1(C)OB(c2ccc3c(c2)C2(c4ccccc4Oc4ccccc42)c2cc(C#N)ccc2-3)OC1(C)C ; Brc1cccc(-c2nc(-c3ccccc3)nc(-c3ccccc3)n2)c1 ; N#Cc1ccc2c(c1)C1(c3ccccc3Oc3ccccc31)c1cc(-c3cccc(-c4nc(-c5ccccc5)nc(-c5ccccc5)n4)c3)ccc1-2]

ETL-2-P2 (12 g, 24.8 mmol) and ETL-2-B (9.6 g, 24.8 mmol) were introduced to tetrahydrofuran (240 ml) under a nitrogen atmosphere, and the mixture was stirred and refluxed. After that, potassium carbonate (10.3 g, 74.5 mmol) dissolved in water (10 ml) was introduced thereto, and, after sufficiently stirring the result, tetrakistriphenyl-phosphinopalladium (0.9 g, 0.7 mmol) was introduced thereto. After reacting for 1 hour, the result was cooled to room temperature, then separated into an organic layer and a water layer, and the organic layer was distilled. This was introduced to chloroform (20 times, 330 mL) again and dissolved therein, and washed twice with water. The organic layer was separated, stirred after introducing anhydrous magnesium sulfate thereto, then filtered, and the filtrate was vacuum distilled. The concentrated compound was recrystallized with chloroform and ethyl acetate to prepare white solid Compound ETL-2 (10.1 g, yield 61%).

MS [M+H]⁺=665

### Preparation Example 8

### Preparation of Compound ETL-3

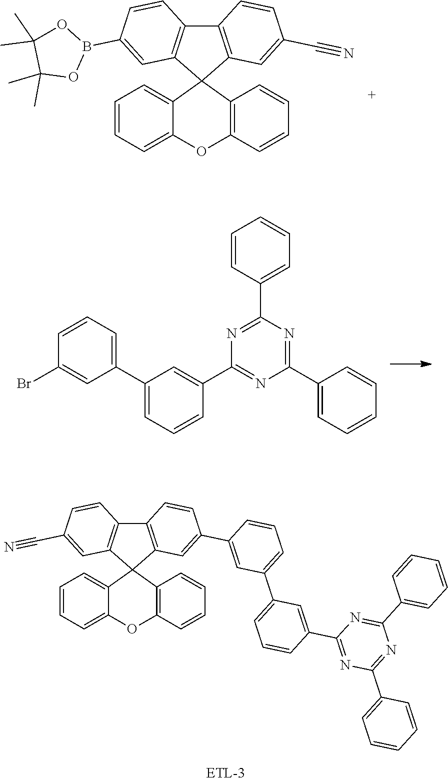 [SMILES: CC1(C)OB(c2ccc3c(c2)C2(c4ccccc4Oc4ccccc42)c2cc(C#N)ccc2-3)OC1(C)C ; Brc1cccc(-c2cccc(-c3nc(-c4ccccc4)nc(-c4ccccc4)n3)c2)c1 ; N#Cc1ccc2c(c1)C1(c3ccccc3Oc3ccccc31)c1cc(-c3cccc(-c4cccc(-c5nc(-c6ccccc6)nc(-c6ccccc6)n5)c4)c3)ccc1-2]

The compound of Chemical Formula ETL-3 was prepared in the same manner as in Preparation of ETL-1 of Preparation Example 6 except that each of the starting materials was used.

MS[M+H]⁺=741

### Preparation Example 9

### Preparation of Compound ETL-4

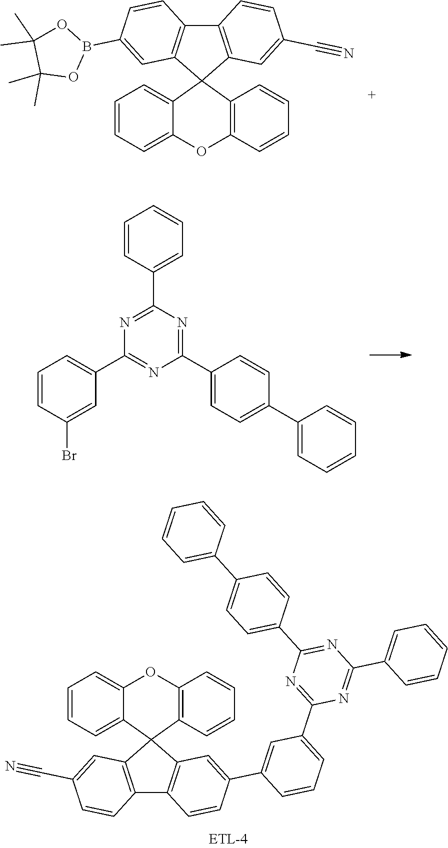 [SMILES: CC1(C)OB(c2ccc3c(c2)C2(c4ccccc4Oc4ccccc42)c2cc(C#N)ccc2-3)OC1(C)C ; Brc1cccc(-c2nc(-c3ccccc3)nc(-c3ccc(-c4ccccc4)cc3)n2)c1 ; N#Cc1ccc2c(c1)C1(c3ccccc3Oc3ccccc31)c1cc(-c3cccc(-c4nc(-c5ccccc5)nc(-c5ccc(-c6ccccc6)cc5)n4)c3)ccc1-2]

The compound of Chemical Formula ETL-4 was prepared in the same manner as in Preparation of ETL-1 of Preparation Example 6 except that each of the starting materials shown above were used.

MS[M+H]⁺=741

### EXPERIMENTAL EXAMPLES

### Comparative Example 1

A glass substrate on which indium tin oxide (ITO) was coated as a thin film to a thickness of 1,000 Å was placed in distilled water containing dissolved detergent and ultrasonically cleaned. Herein, a product of Fischer Co. was used as the detergent, and as the distilled water, distilled water filtered twice with a filter manufactured by Millipore Co. was used. After the ITO was cleaned for 30 minutes, ultrasonic cleaning was repeated twice using distilled water for 10 minutes. After the cleaning with distilled water was finished, the substrate was ultrasonically cleaned with solvents of isopropyl alcohol, acetone and methanol, then dried, and then transferred to a plasma cleaner. The substrate was cleaned for 5 minutes using nitrogen plasma, and then transferred to a vacuum deposition apparatus.

On the transparent ITO electrode prepared as above, the following HT1 compound and HI1 compound in a weight ratio (HT1:HI1) of 100:6 were vacuum deposited to a thickness of 100 Å to form a hole injection layer. On the hole injection layer, the following HT1 compound was vacuum deposited to a thickness of 1,100 Å to form a hole transfer layer. An electron blocking layer was formed on the hole transfer layer by vacuum depositing the following EBL-1 compound prepared above to a thickness of 50 Å. On the electron blocking layer, the following HOST-1 compound and the following BD compound in a weight ratio (HOST-1:BD) of 96:4 were vacuum deposited to a thickness of 200 Å to form a light emitting layer. A hole blocking layer was formed on the light emitting layer by vacuum depositing the following HBL compound to a thickness of 50 Å. On the hole blocking layer, the following ETL-1 compound prepared above and the following LiQ compound in a weight ratio of 1:1 were vacuum deposited to a thickness of 310 Å to form an electron transfer layer. On the electron transfer layer, magnesium and silver in a weight ratio (magnesium:silver) of 9:1 were deposited to a thickness of 120 Å and then aluminum was deposited to a thickness of 1,000 Å to form a cathode.

In the above-described process, the deposition rates of the silver (Ag) and the aluminum were maintained at 0.1 Å/sec and 2 Å/sec, respectively, and the degree of vacuum during the deposition was maintained at 5×10⁻⁸ torr to 1×10⁻⁷ torr, and as a result, an organic light emitting device was manufactured.

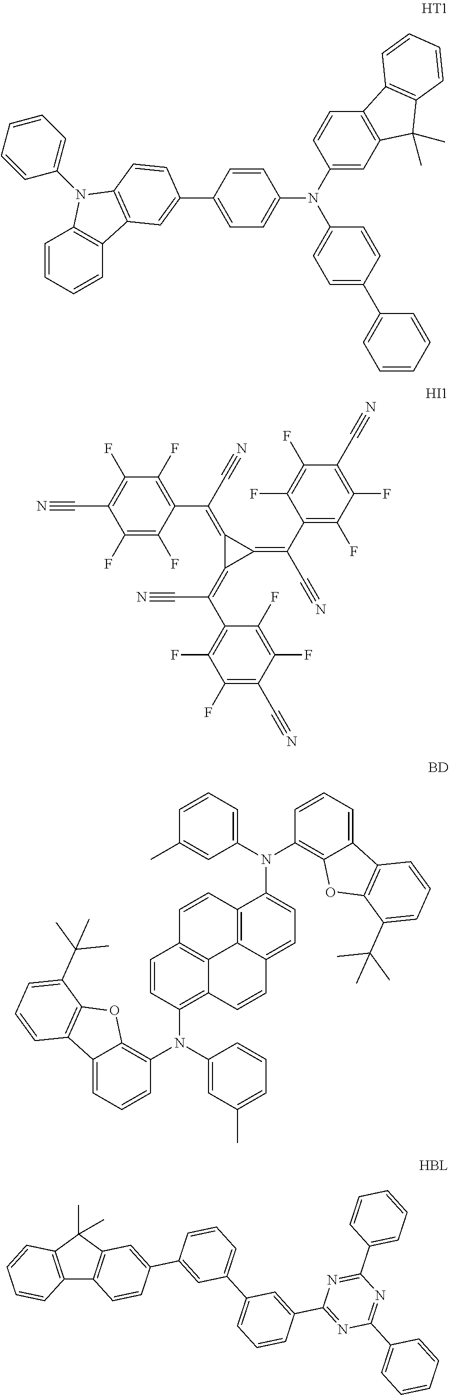

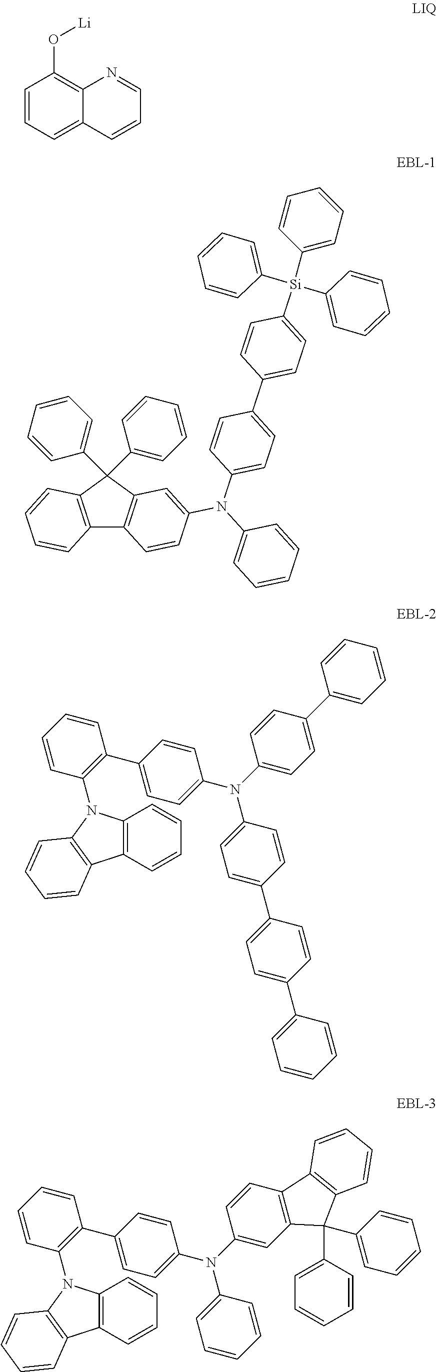

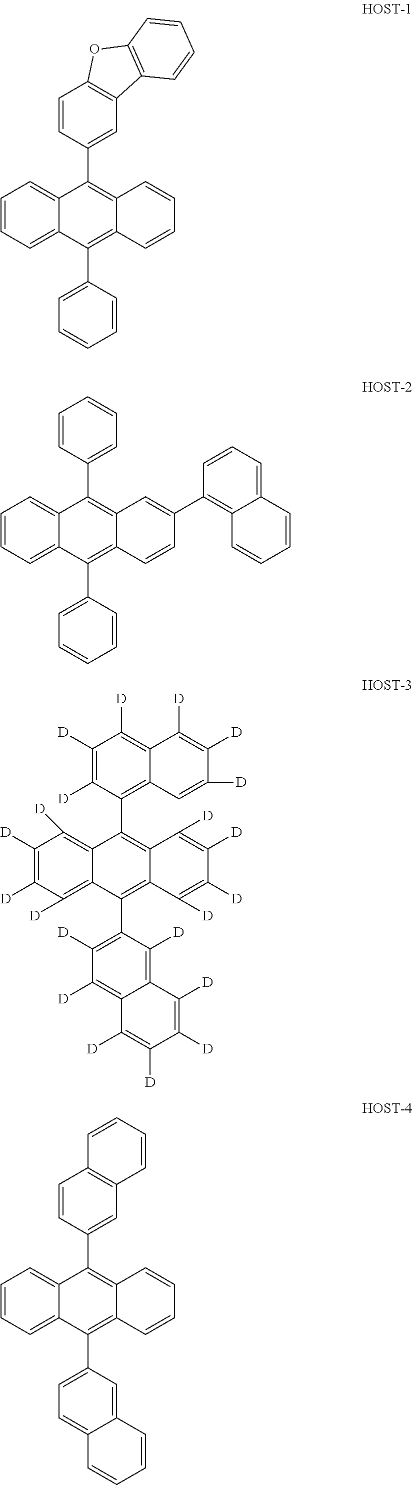

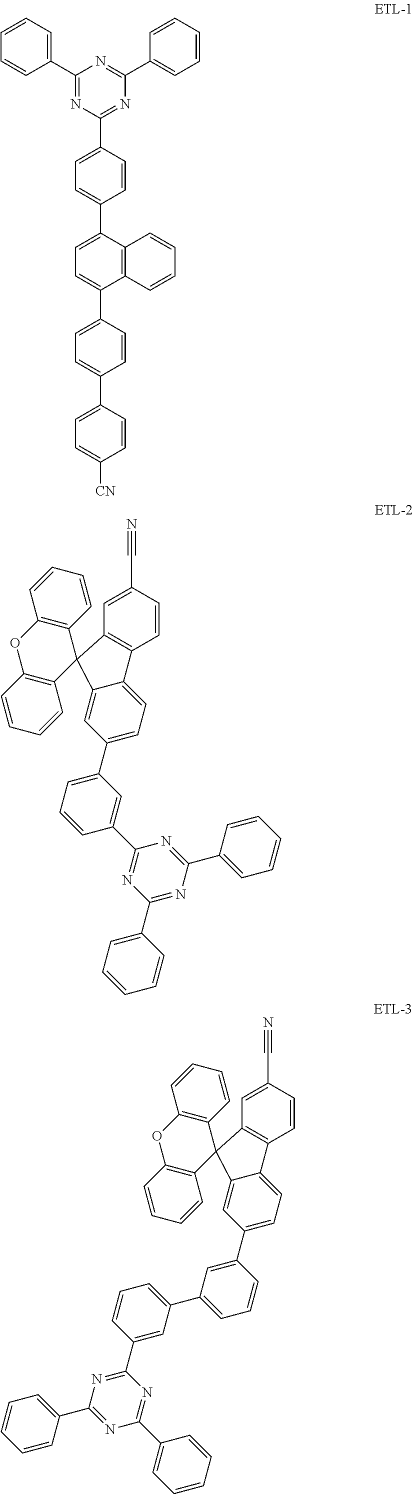 [SMILES: N#Cc1ccc(-c2ccc(-c3ccc(-c4ccc(-c5nc(-c6ccccc6)nc(-c6ccccc6)n5)cc4)c4ccccc34)cc2)cc1 ; N#Cc1ccc2c(c1)C1(c3ccccc3Oc3ccccc31)c1cc(-c3cccc(-c4nc(-c5ccccc5)nc(-c5ccccc5)n4)c3)ccc1-2 ; N#Cc1ccc2c(c1)C1(c3ccccc3Oc3ccccc31)c1cc(-c3cccc(-c4cccc(-c5nc(-c6ccccc6)nc(-c6ccccc6)n5)c4)c3)ccc1-2]

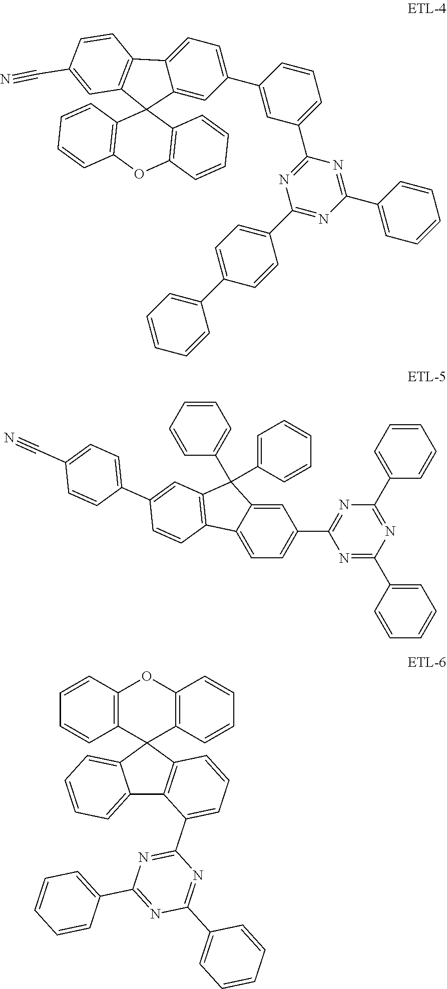 [SMILES: N#Cc1ccc2c(c1)C1(c3ccccc3Oc3ccccc31)c1cc(-c3cccc(-c4nc(-c5ccccc5)nc(-c5ccc(-c6ccccc6)cc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; c1ccc(-c2nc(-c3ccccc3)nc(-c3cccc4c3-c3ccccc3C43c4ccccc4Oc4ccccc43)n2)cc1]

### Comparative Examples 2 to 9 and Examples 1 to 12

Organic light emitting devices were manufactured in the same manner as in Comparative Example 1 except that compounds described in the following Table 1 were used instead of EBL-1, HOST-1 and ETL-1.

For each of the organic light emitting devices manufactured in Comparative Examples 1 to 9 and Examples 1 to 12, efficiency was measured at current density of 10 mA/cm², and the results are shown in the following Table 1.

TABLE 1

| | Structure/Material | | | |
|---|---|---|---|---|
| | Electron | Light | Electron | |
| Experimental | Blocking | Emitting | Transfer | |
| Example | Layer | Layer | Layer | Cd/A |
| Comparative | EBL-1 | HOST-1 | ETL-1 | 6.92 |
| Example 1 | | | | |
| Comparative | EBL-1 | HOST-2 | ETL-1 | 7.49 |
| Example 2 | | | | |
| Comparative | EBL-2 | HOST-2 | ETL-1 | 7.28 |
| Example 3 | | | | |
| Comparative | EBL-3 | HOST-2 | ETL-1 | 7.39 |
| Example 4 | | | | |
| Comparative | EBL-1 | HOST-1 | ETL-5 | 6.64 |
| Example 5 | | | | |
| Comparative | EBL-1 | HOST-2 | ETL-6 | 6.6 |
| Example 6 | | | | |
| Comparative | EBL-2 | HOST-1 | ETL-1 | 6.52 |
| Example 7 | | | | |
| Comparative | EBL-2 | HOST-1 | ETL-3 | 7.09 |
| Example 8 | | | | |
| Comparative | EBL-1 | HOST-1 | ETL-4 | 7.34 |
| Example 9 | | | | |
| Example 1 | EBL-2 | HOST-2 | ETL-2 | 8.02 |
| Example 2 | EBL-2 | HOST-2 | ETL-3 | 8.04 |
| Example 3 | EBL-2 | HOST-2 | ETL-4 | 7.74 |
| Example 4 | EBL-2 | HOST-3 | ETL-2 | 7.99 |
| Example 5 | EBL-2 | HOST-3 | ETL-3 | 8 |
| Example 6 | EBL-3 | HOST-2 | ETL-2 | 8.14 |
| Example 7 | EBL-3 | HOST-2 | ETL-3 | 8.15 |
| Example 8 | EBL-3 | HOST-2 | ETL-4 | 7.84 |
| Example 9 | EBL-3 | HOST-3 | ETL-2 | 8.2 |
| Example 10 | EBL-3 | HOST-3 | ETL-3 | 8.2 |
| Example 11 | EBL-3 | HOST-3 | ETL-4 | 7.84 |
| Example 12 | EBL-3 | HOST-4 | ETL-2 | 8.12 |

From the experimental results of Table 1, it was identified that the devices of Examples 1 to 12 of the present disclosure had superior efficiency compared to the devices of Comparative Examples 1 to 9 not including the compounds corresponding to Chemical Formulae 1 to 3 of the present application in one or more of the electron blocking layer, the light emitting layer and the electron transfer layer.

Specifically, Comparative Examples 1 and 5 did not include the compounds corresponding to Chemical Formulae 1 to 3 of the present application, Comparative Example 2 did not include the compounds corresponding to Chemical Formulae 1 and 3 of the present application, Comparative Examples 3 and 4 did not include the compound corresponding to Chemical Formula 3 of the present application, Comparative Example 6 did not include the compounds corresponding to Chemical Formulae 1 and 3 of the present application, Comparative Example 7 did not include the compounds corresponding to Chemical Formulae 2 and 3 of the present application, Comparative Example 8 did not include the compound corresponding to Chemical Formula 2 of the present application, and Comparative Example 9 did not include the compounds corresponding to Chemical Formulae 1 and 2 of the present application. It was seen that Examples 1 to 12 of the present application had higher device efficiency compared to Comparative Examples 1 to 9.

1. An organic light emitting device comprising:
an anode;
a cathode;
a light emitting layer provided between the anode and the cathode and including a compound of the following Chemical Formula 2;
a first organic material layer provided between the anode and the light emitting layer and including a compound of the following Chemical Formula 1; and
a second organic material layer provided between the light emitting layer and the cathode and including a compound of the following Chemical Formula 3: 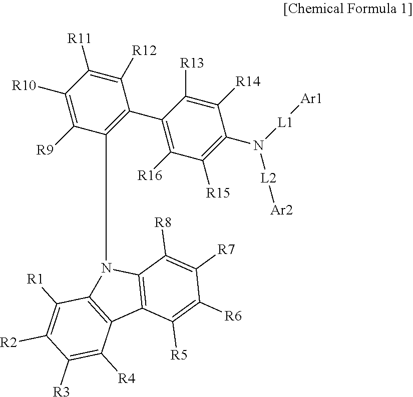
wherein, in Chemical Formula 1;
L1 and L2 are the same as or different from each other, and each independently is a direct bond or a substituted or unsubstituted arylene group;
Ar1 and Ar2 are the same as or different from each other, and each independently is hydrogen, deuterium, a halogen group, a cyano group, a substituted or unsubstituted silyl group, a substituted or unsubstituted alkyl group, a substituted or unsubstituted cycloalkyl group, or a substituted or unsubstituted aryl group;
R1 to R8 are the same as or different from each other, and each independently is hydrogen or deuterium, or bond to adjacent groups to form a substituted or unsubstituted ring; and
R9 to R16 are the same as or different from each other, and each independently is hydrogen or deuteriums; 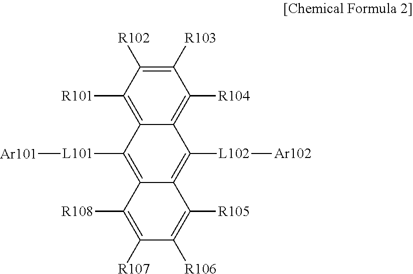
wherein in Chemical Formula 2:
L101 and L102 are the same as or different from each other, and each independently is a direct bond, a substituted or unsubstituted alkylene group, or a substituted or unsubstituted arylene group; and
Ar101, Ar102 and R101 to R108 are the same as or different from each other, and each independently is hydrogen, deuterium, a substituted or unsubstituted alkyl group, or a substituted or unsubstituted aryl group; 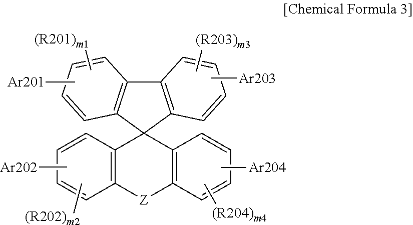
wherein in Chemical Formula 3:
Z is O or S;
R201 to R204 are the same as or different from each other, and each independently is hydrogen, deuterium, a halogen group, a cyano group, a substituted or unsubstituted alkyl group, a substituted or unsubstituted cycloalkyl group, a substituted or unsubstituted aryl group, or a substituted or unsubstituted heterocyclic group;
m1 to m4 are each an integer of 0 to 3, and when m1 to m4 are each 2 or greater, substituents in the parentheses are the same as or different from each other;
Ar201 and Ar202 are the same as or different from each other, at least one of Ar201 and Ar202 is -L201-CN, and any remaining is hydrogen;
L201 is a direct bond or a substituted or unsubstituted arylene group; and
Ar203 and Ar204 are the same as or different from each other, at least one of Ar203 and Ar204 is the following Chemical Formula A-1, and any remaining is hydrogen; 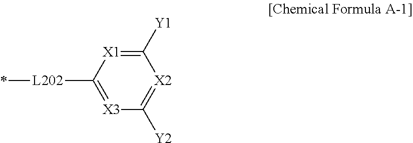
wherein in Chemical Formula A-1:
X1 to X3 are each N or CY3, and at least two of X1 to X3 are N;
Y1 to Y3 are the same as or different from each other, and each independently is hydrogen, deuterium, a substituted or unsubstituted alkyl group, a substituted or unsubstituted hydrocarbon ring group, or a substituted or unsubstituted heterocyclic group;
L202 is a direct bond or a substituted or unsubstituted arylene group; and
* means a bonding position.

2. The organic light emitting device of claim 1, wherein Chemical Formula 1 is any one of the following Chemical Formulae 1-1 to 1-4: 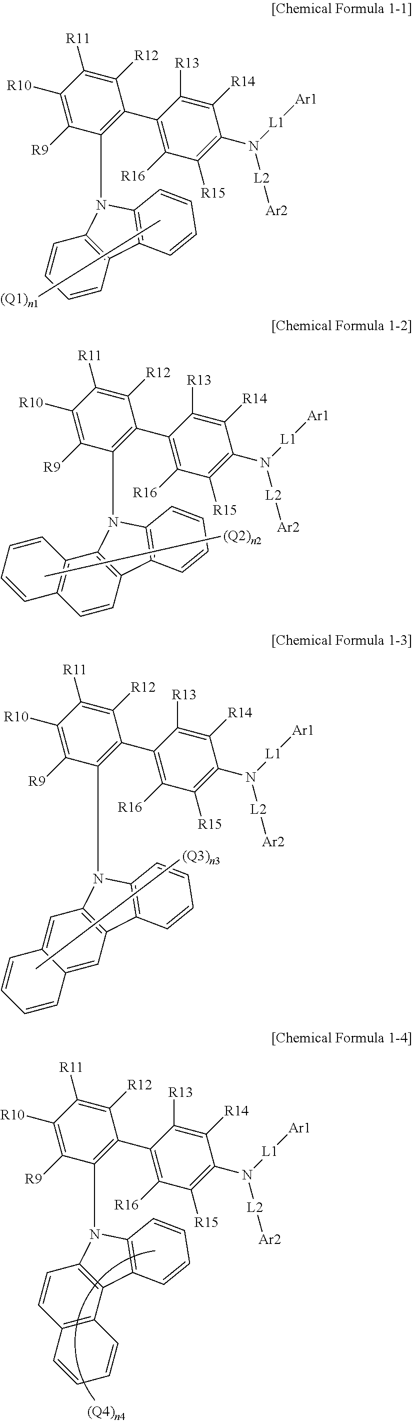
wherein in Chemical Formulae 1-1 to 1-4:
R9 to R16, L1, L2, Ar1 and Ar2 have the same definitions as in Chemical Formula 1;
Q1 to Q4 are the same as or different from each other, and each independently; is hydrogen or deuterium;
n1 is an integer of 0 to 8, and n2 to n4 are each an integer of 0 to 10; and
when n1 to n4 are each 2 or greater, substituents in the parentheses are the same as or different from each other.

3. The organic light emitting device of claim 1, wherein Chemical Formula 3 is any one of the following Chemical Formulae 3-1 to 3-4: 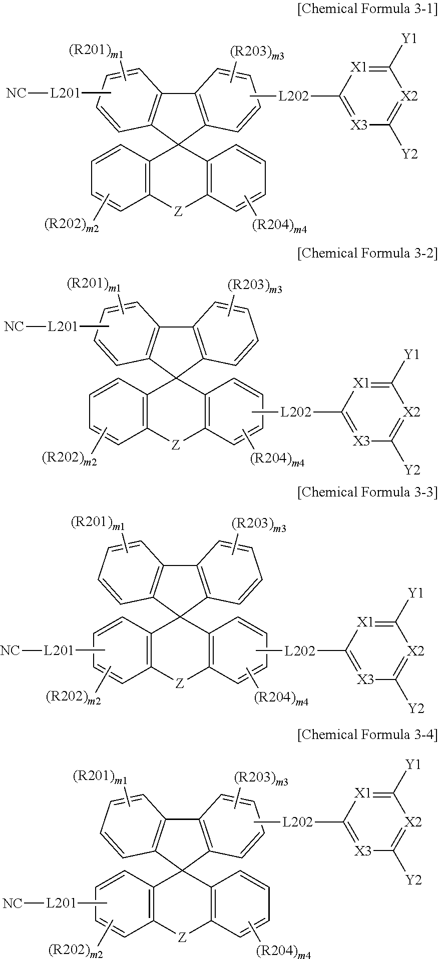
wherein in Chemical Formulae 3-1 to 3-4:
Z, R201 to R204, m1 to m4, L201, L202, X1 to X3, Y1 and Y2 have the same definitions as in Chemical Formula 3.

4. The organic light emitting device of claim 1, wherein the compound of Chemical Formula 1 is any one compound selected from among the following compounds:

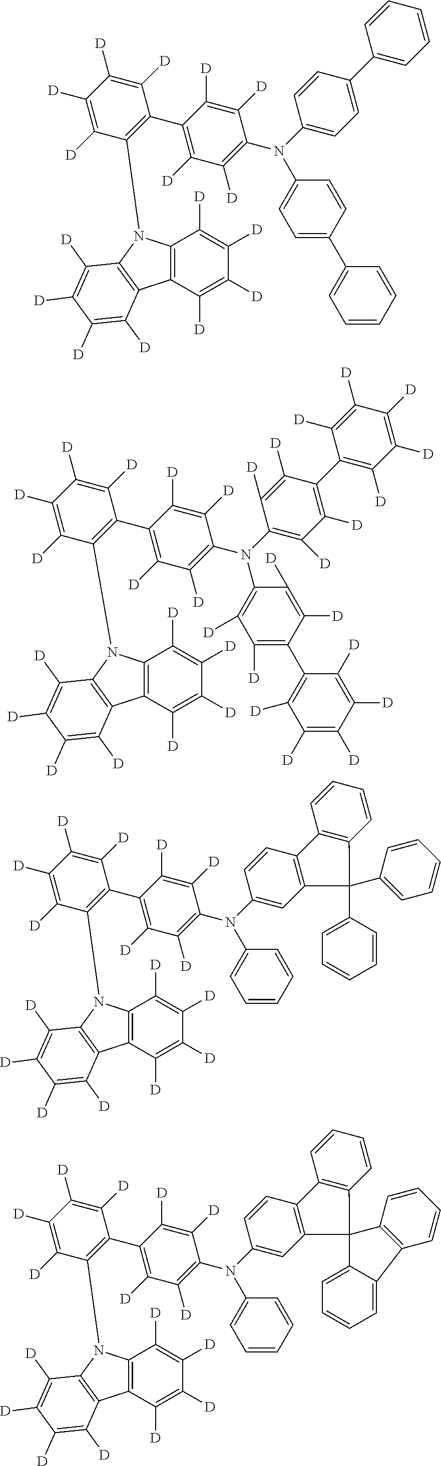

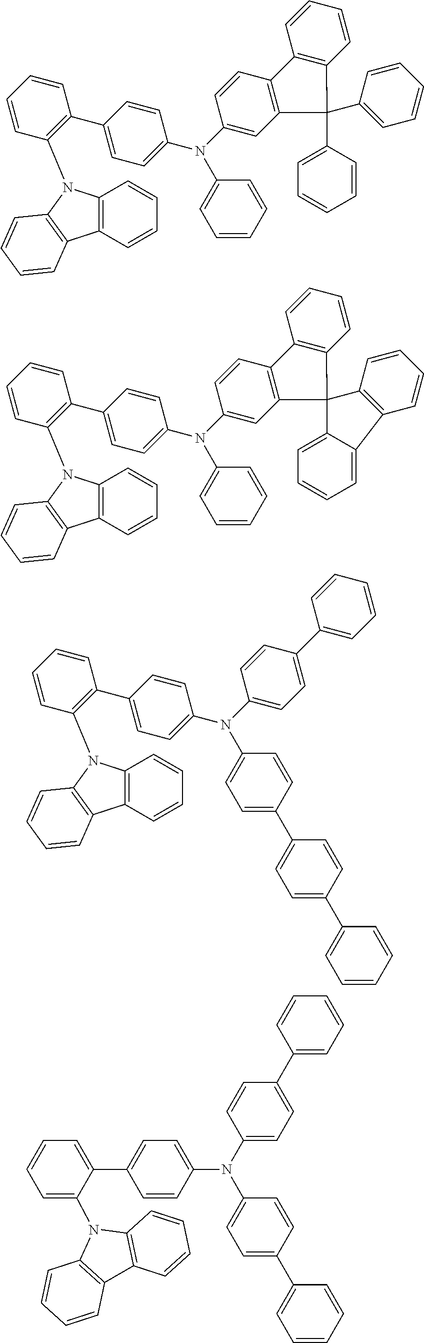

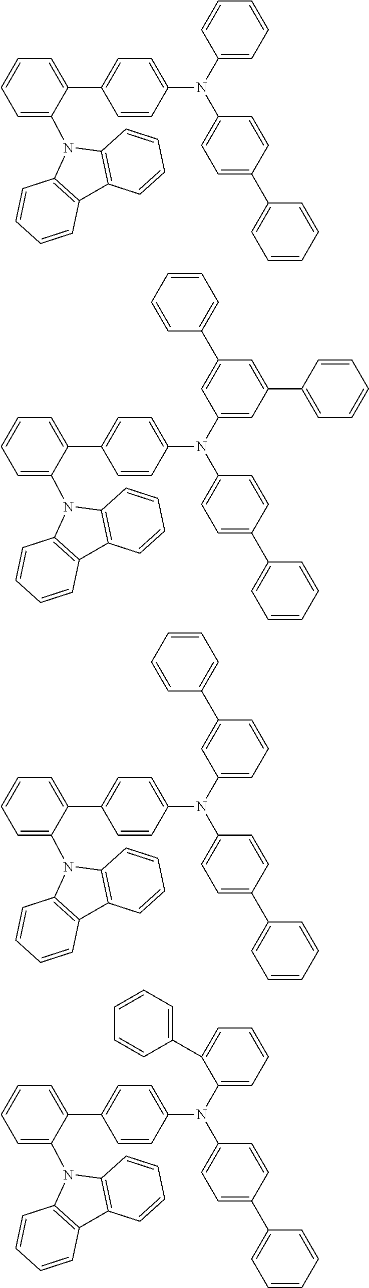 [SMILES: c1ccc(-c2ccc(N(c3ccccc3)c3ccc(-c4ccccc4-n4c5ccccc5c5ccccc54)cc3)cc2)cc1 ; c1ccc(-c2ccc(N(c3ccc(-c4ccccc4-n4c5ccccc5c5ccccc54)cc3)c3cc(-c4ccccc4)cc(-c4ccccc4)c3)cc2)cc1 ; c1ccc(-c2ccc(N(c3ccc(-c4ccccc4-n4c5ccccc5c5ccccc54)cc3)c3cccc(-c4ccccc4)c3)cc2)cc1 ; c1ccc(-c2ccc(N(c3ccc(-c4ccccc4-n4c5ccccc5c5ccccc54)cc3)c3ccccc3-c3ccccc3)cc2)cc1]

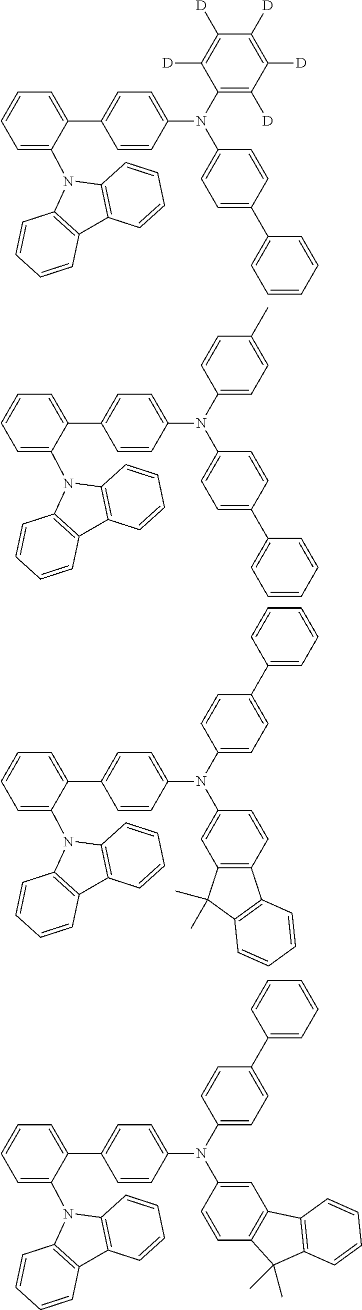

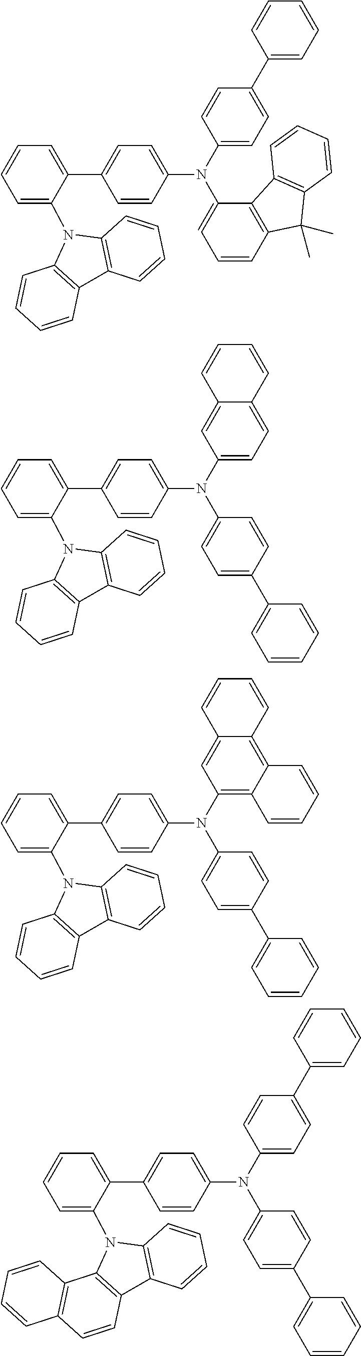

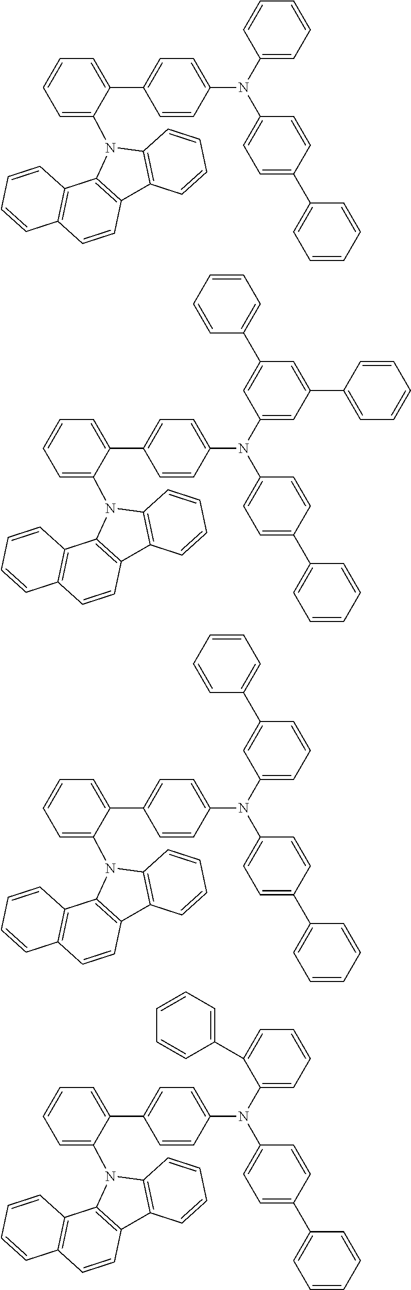 [SMILES: c1ccc(-c2ccc(N(c3ccccc3)c3ccc(-c4ccccc4-n4c5ccccc5c5ccc6ccccc6c54)cc3)cc2)cc1 ; c1ccc(-c2ccc(N(c3ccc(-c4ccccc4-n4c5ccccc5c5ccc6ccccc6c54)cc3)c3cc(-c4ccccc4)cc(-c4ccccc4)c3)cc2)cc1 ; c1ccc(-c2ccc(N(c3ccc(-c4ccccc4-n4c5ccccc5c5ccc6ccccc6c54)cc3)c3cccc(-c4ccccc4)c3)cc2)cc1 ; c1ccc(-c2ccc(N(c3ccc(-c4ccccc4-n4c5ccccc5c5ccc6ccccc6c54)cc3)c3ccccc3-c3ccccc3)cc2)cc1]

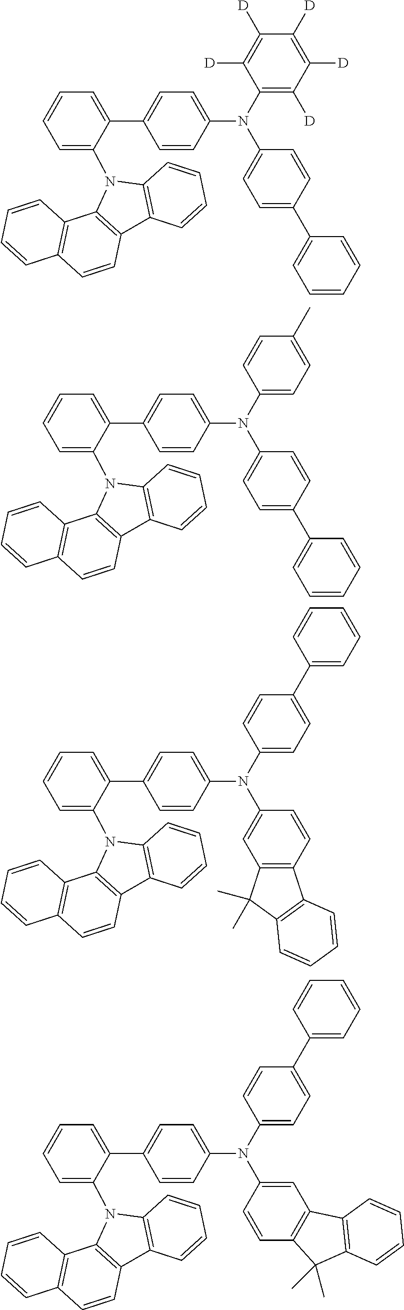

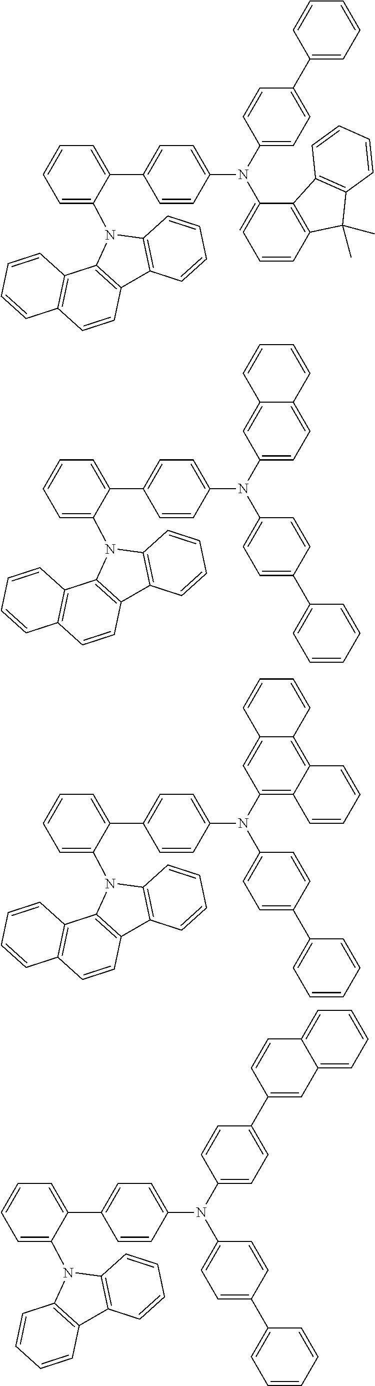

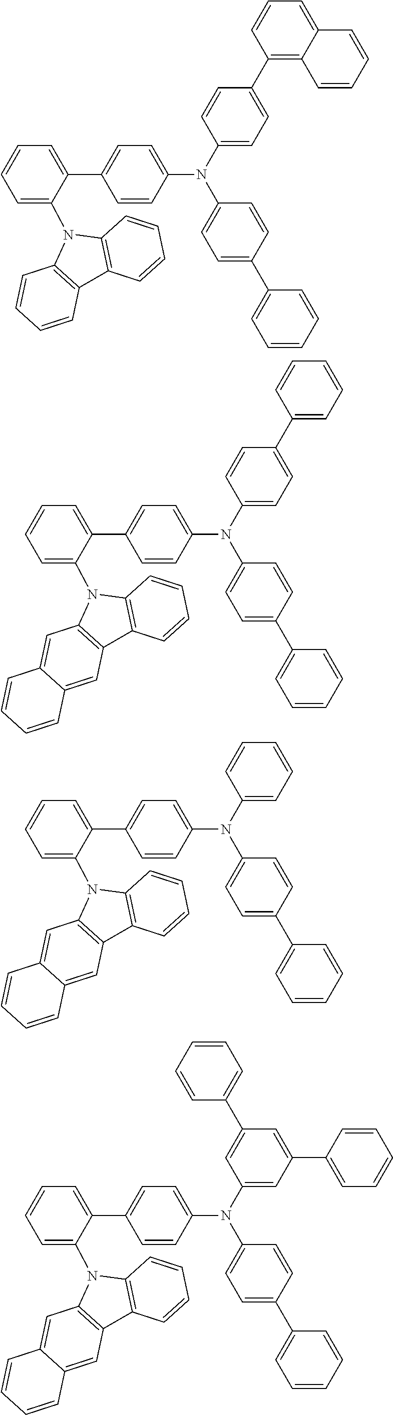

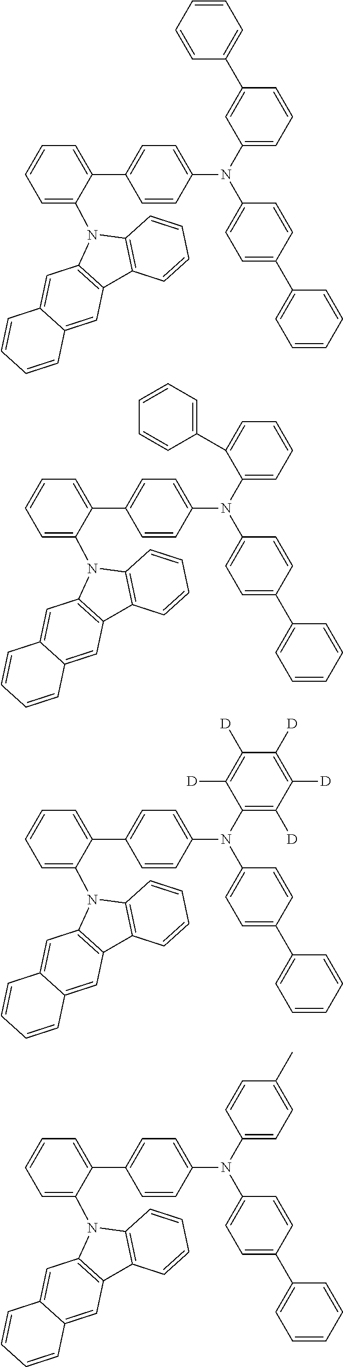

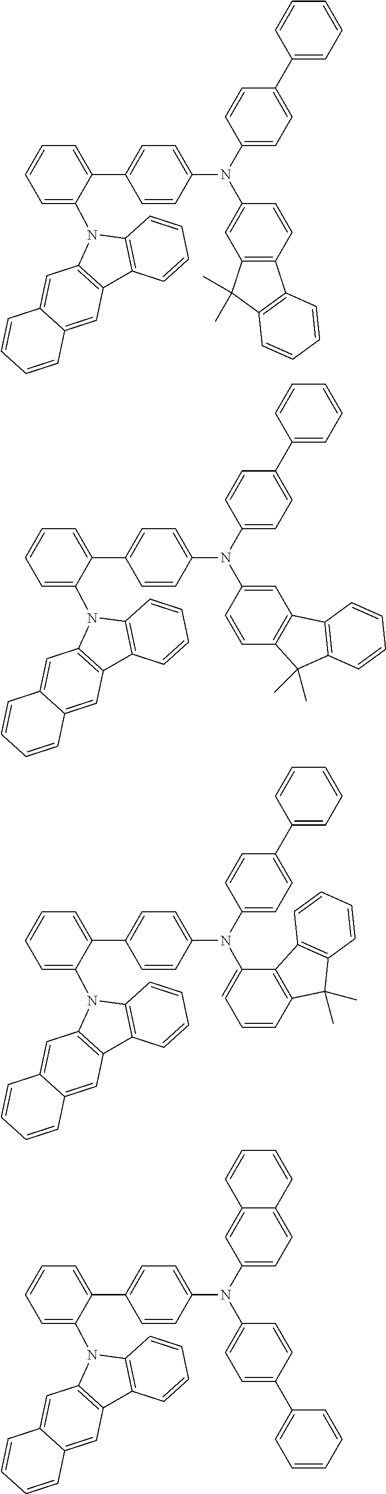

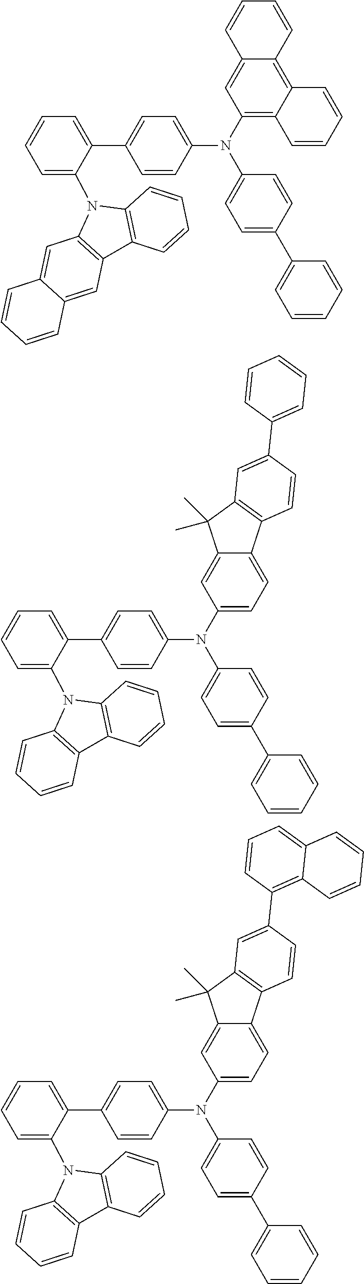

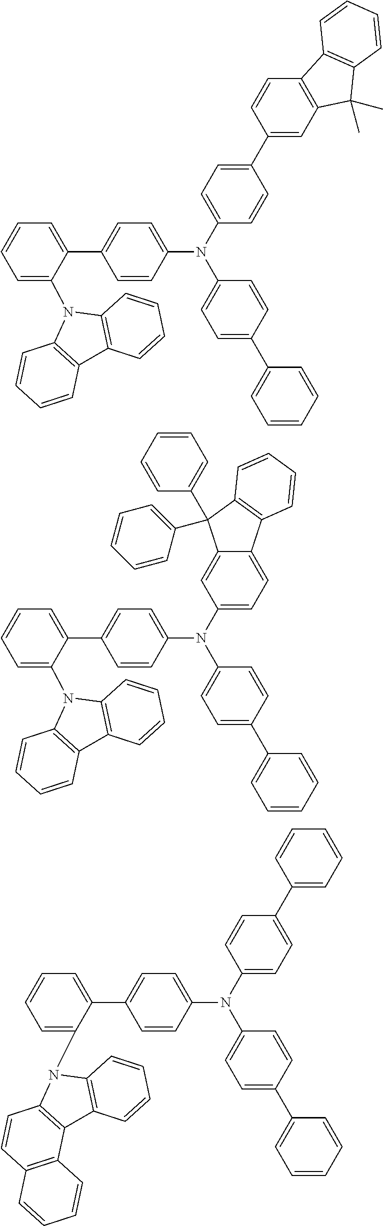

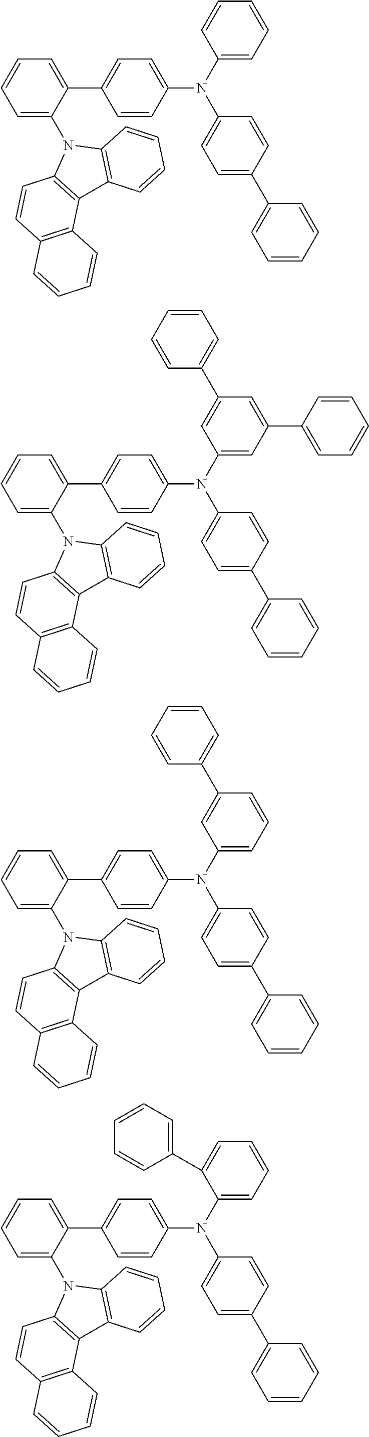 [SMILES: c1ccc(-c2ccc(N(c3ccccc3)c3ccc(-c4ccccc4-n4c5ccccc5c5c6ccccc6ccc54)cc3)cc2)cc1 ; c1ccc(-c2ccc(N(c3ccc(-c4ccccc4-n4c5ccccc5c5c6ccccc6ccc54)cc3)c3cc(-c4ccccc4)cc(-c4ccccc4)c3)cc2)cc1 ; c1ccc(-c2ccc(N(c3ccc(-c4ccccc4-n4c5ccccc5c5c6ccccc6ccc54)cc3)c3cccc(-c4ccccc4)c3)cc2)cc1 ; c1ccc(-c2ccc(N(c3ccc(-c4ccccc4-n4c5ccccc5c5c6ccccc6ccc54)cc3)c3ccccc3-c3ccccc3)cc2)cc1]

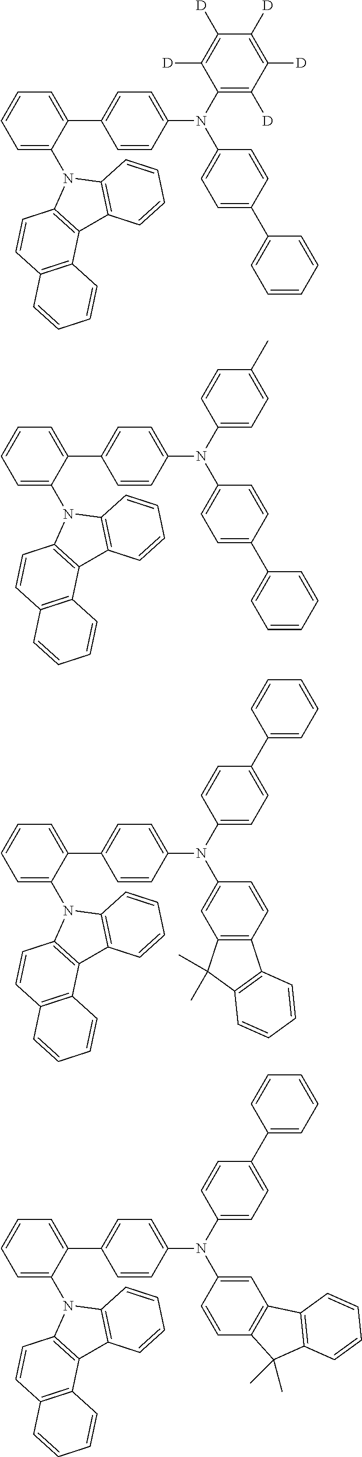

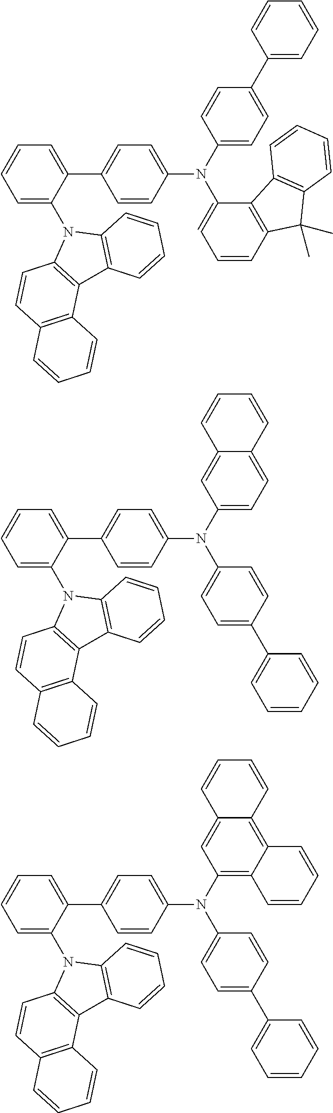 [SMILES: CC1(C)c2ccccc2-c2c(N(c3ccc(-c4ccccc4)cc3)c3ccc(-c4ccccc4-n4c5ccccc5c5c6ccccc6ccc54)cc3)cccc21 ; c1ccc(-c2ccc(N(c3ccc(-c4ccccc4-n4c5ccccc5c5c6ccccc6ccc54)cc3)c3ccc4ccccc4c3)cc2)cc1 ; c1ccc(-c2ccc(N(c3ccc(-c4ccccc4-n4c5ccccc5c5c6ccccc6ccc54)cc3)c3cc4ccccc4c4ccccc34)cc2)cc1]

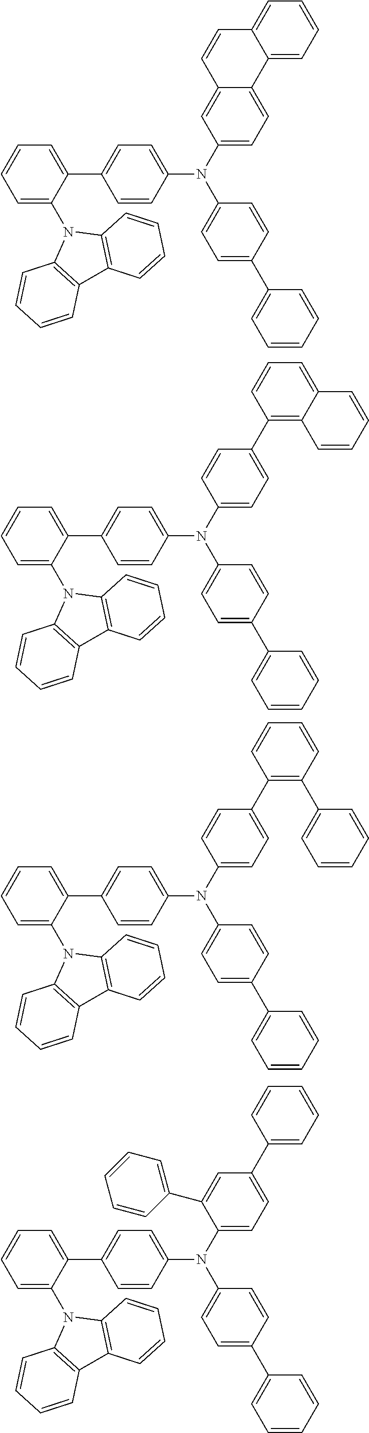

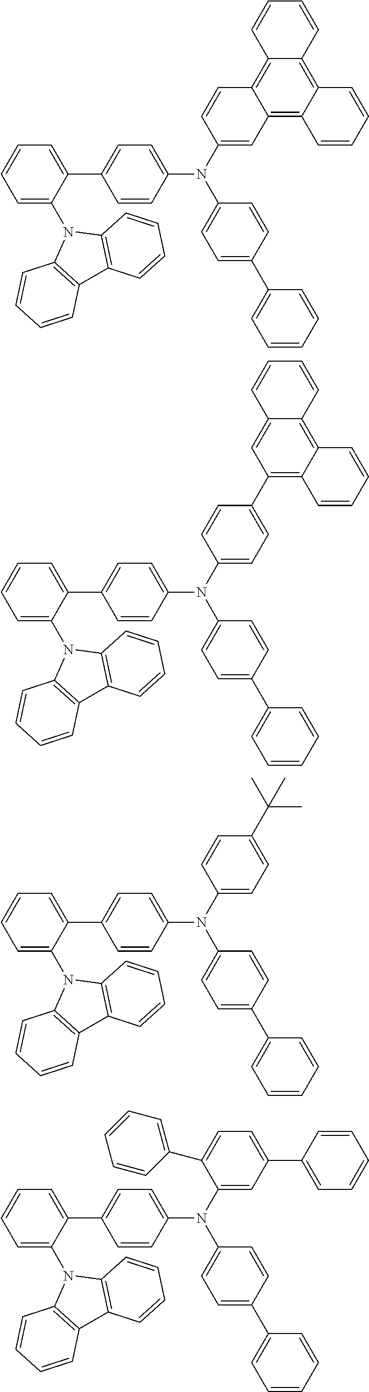

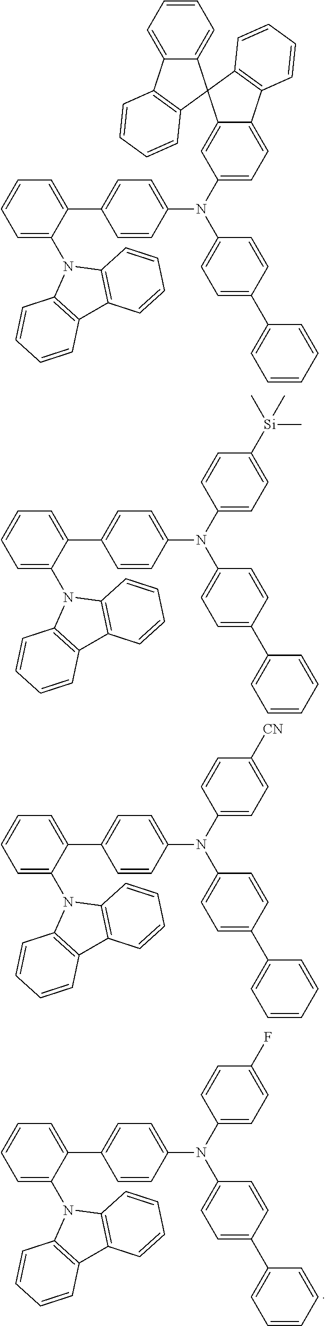

5. The organic light emitting device of claim 1, wherein the compound of Chemical Formula 2 is any one compound selected from among the following compounds:

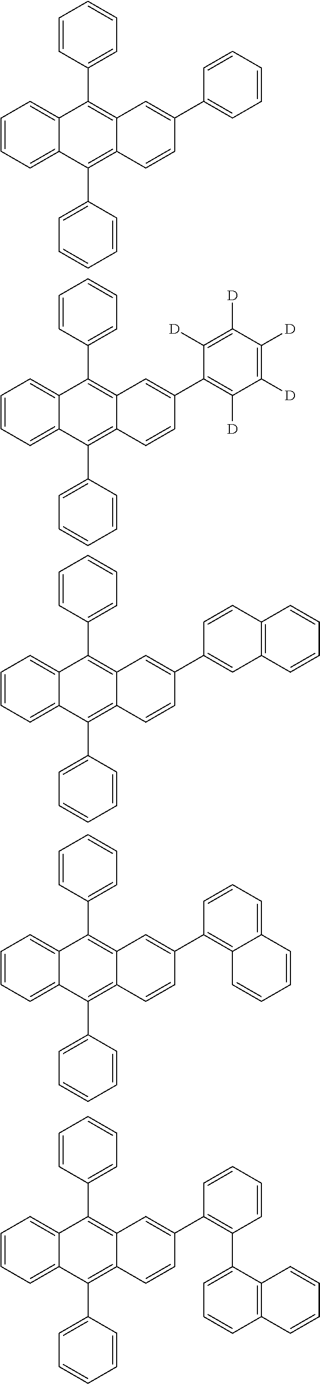

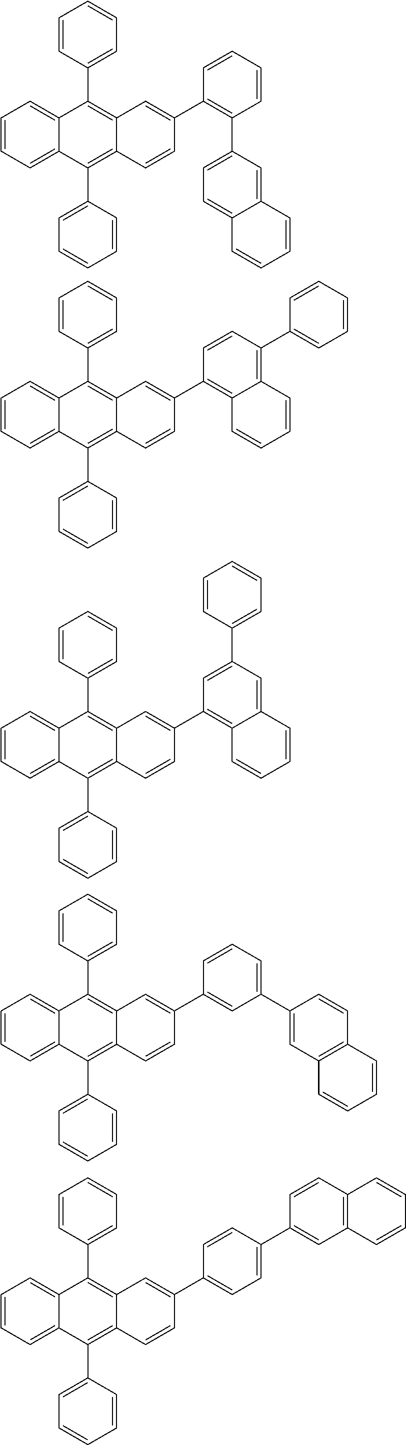

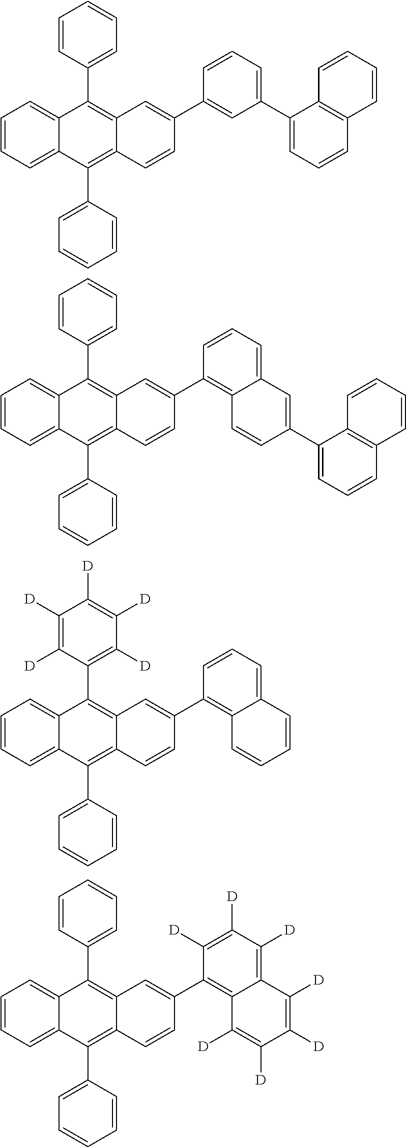

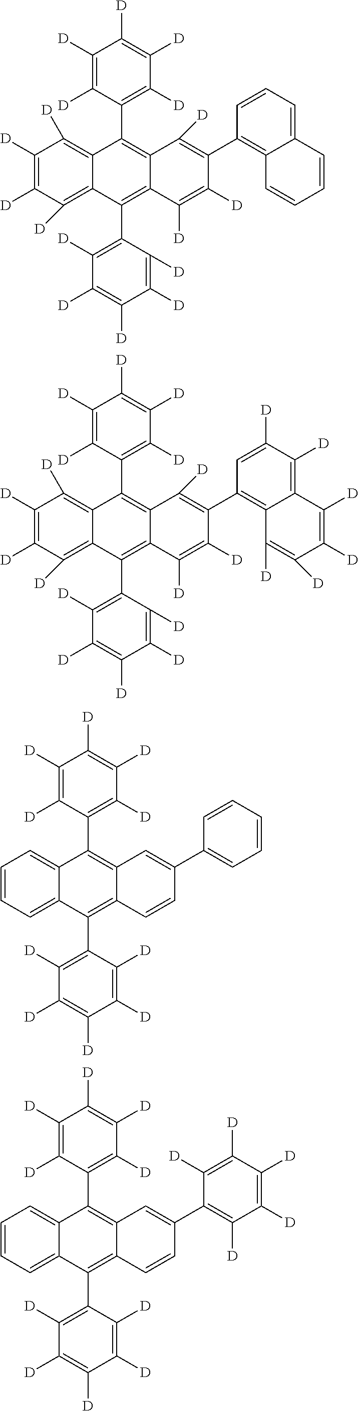

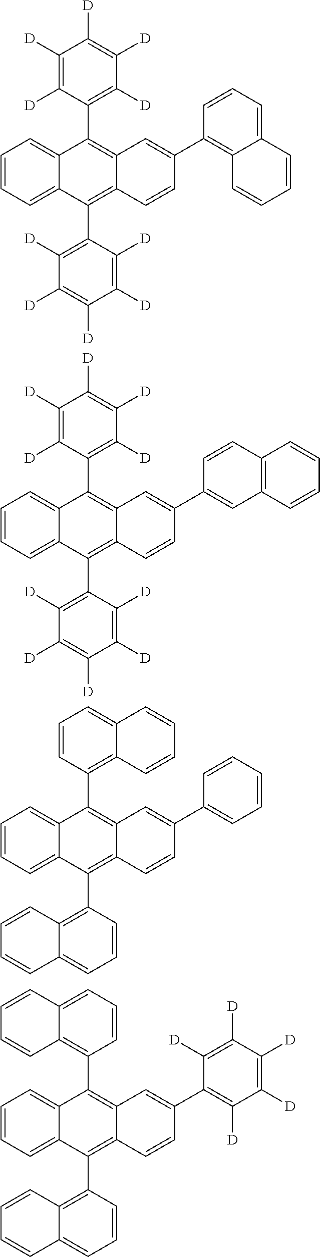

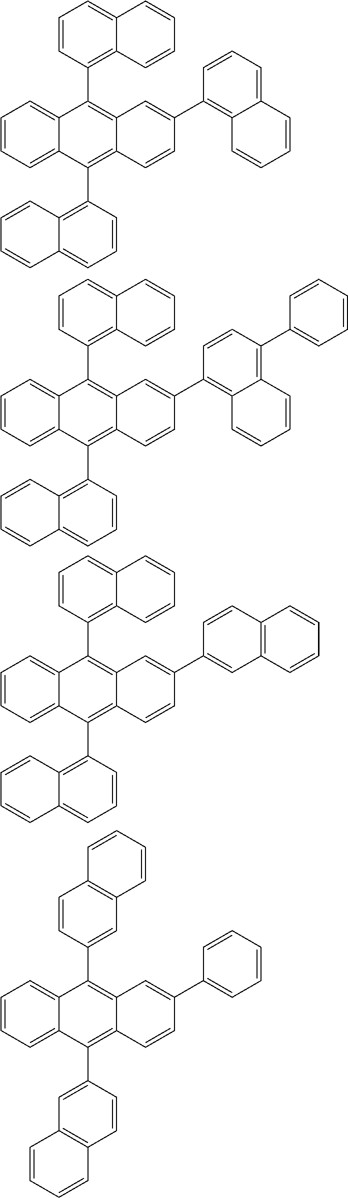

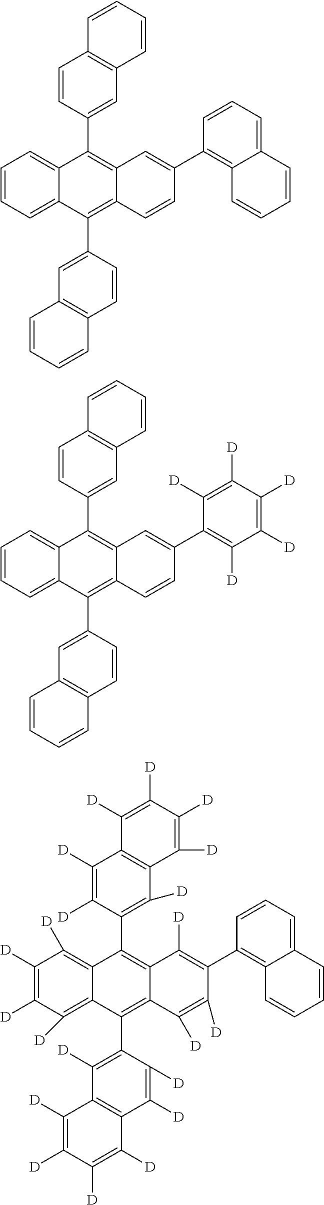

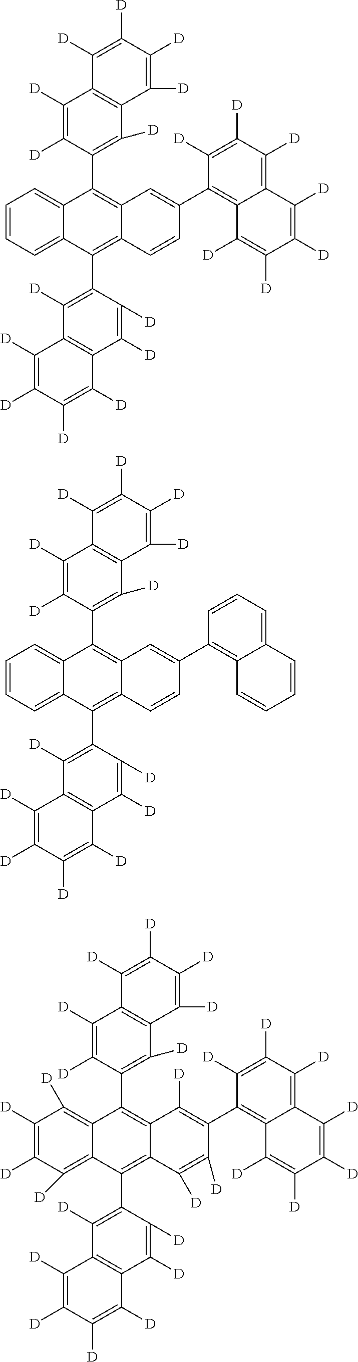

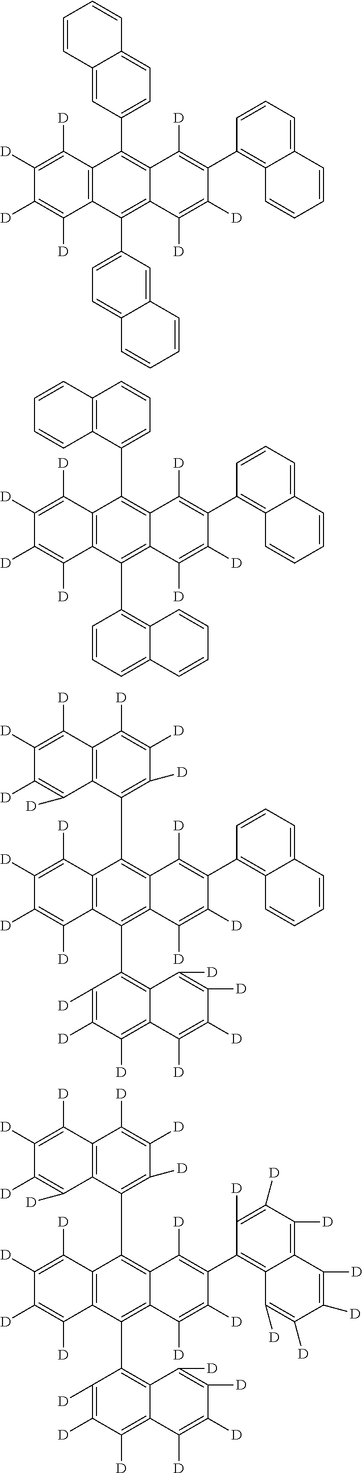

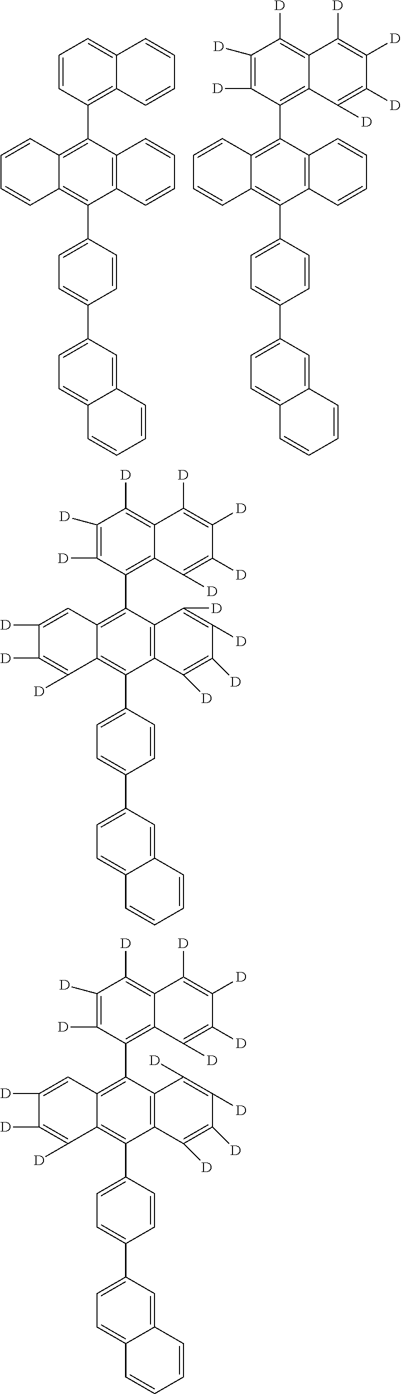

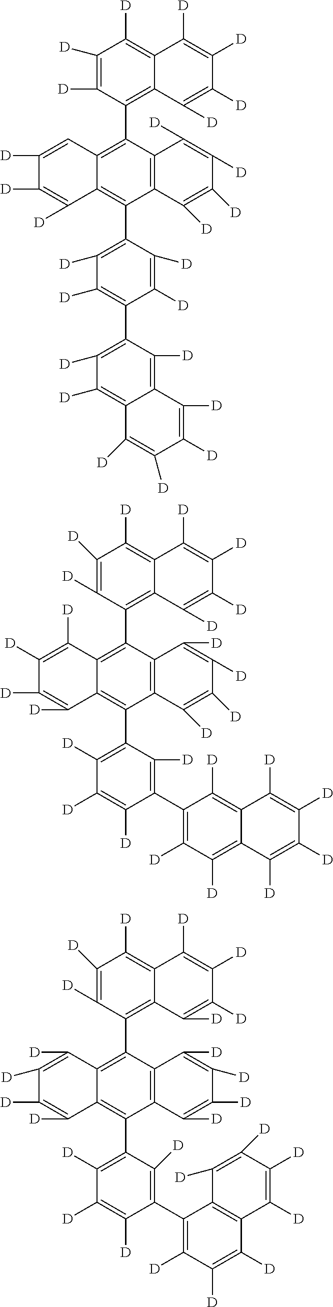

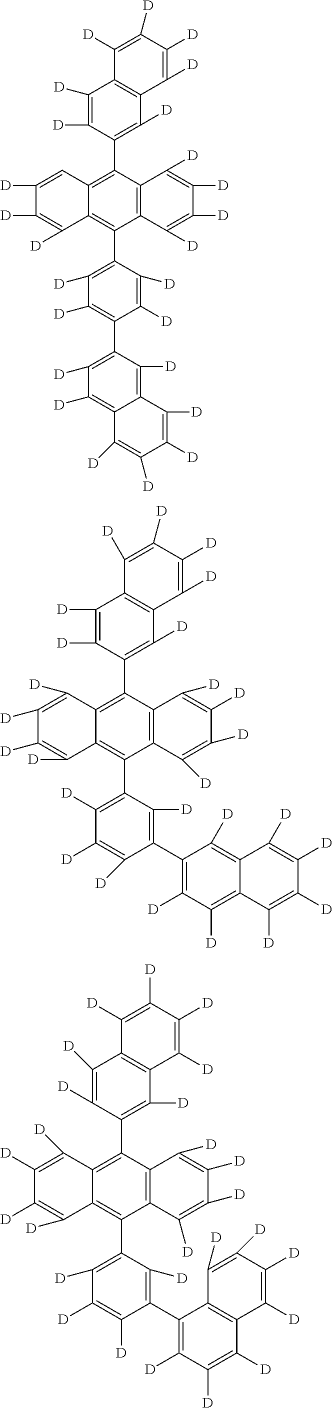

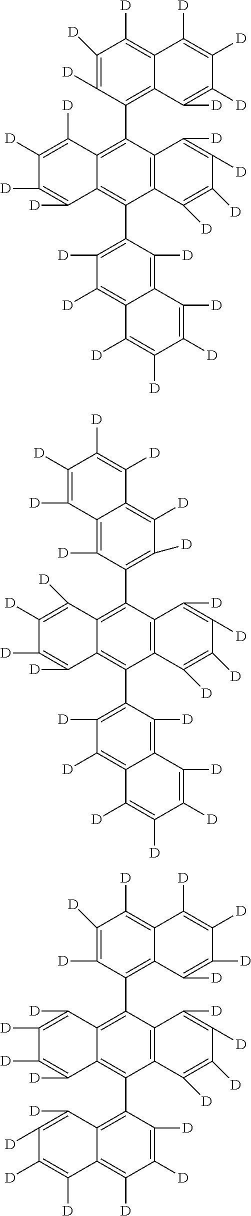

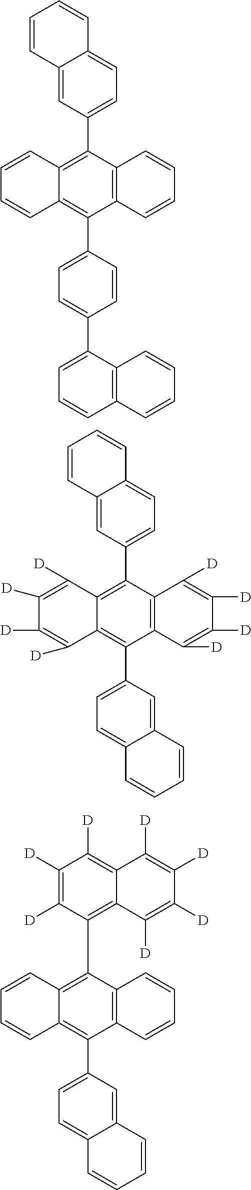

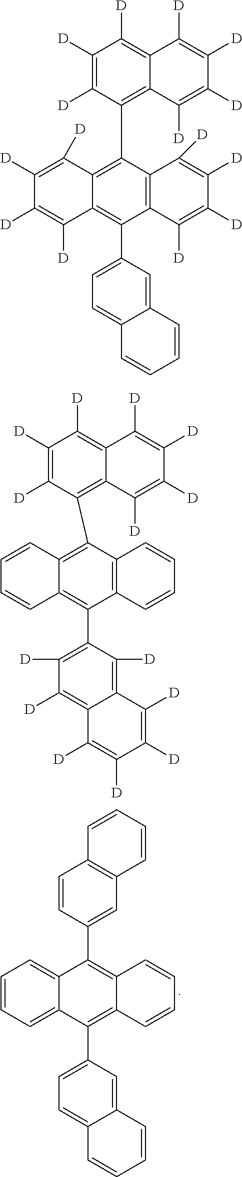

6. The organic light emitting device of claim 1, wherein the compound of Chemical Formula 3 is any one compound selected from among the following compounds:

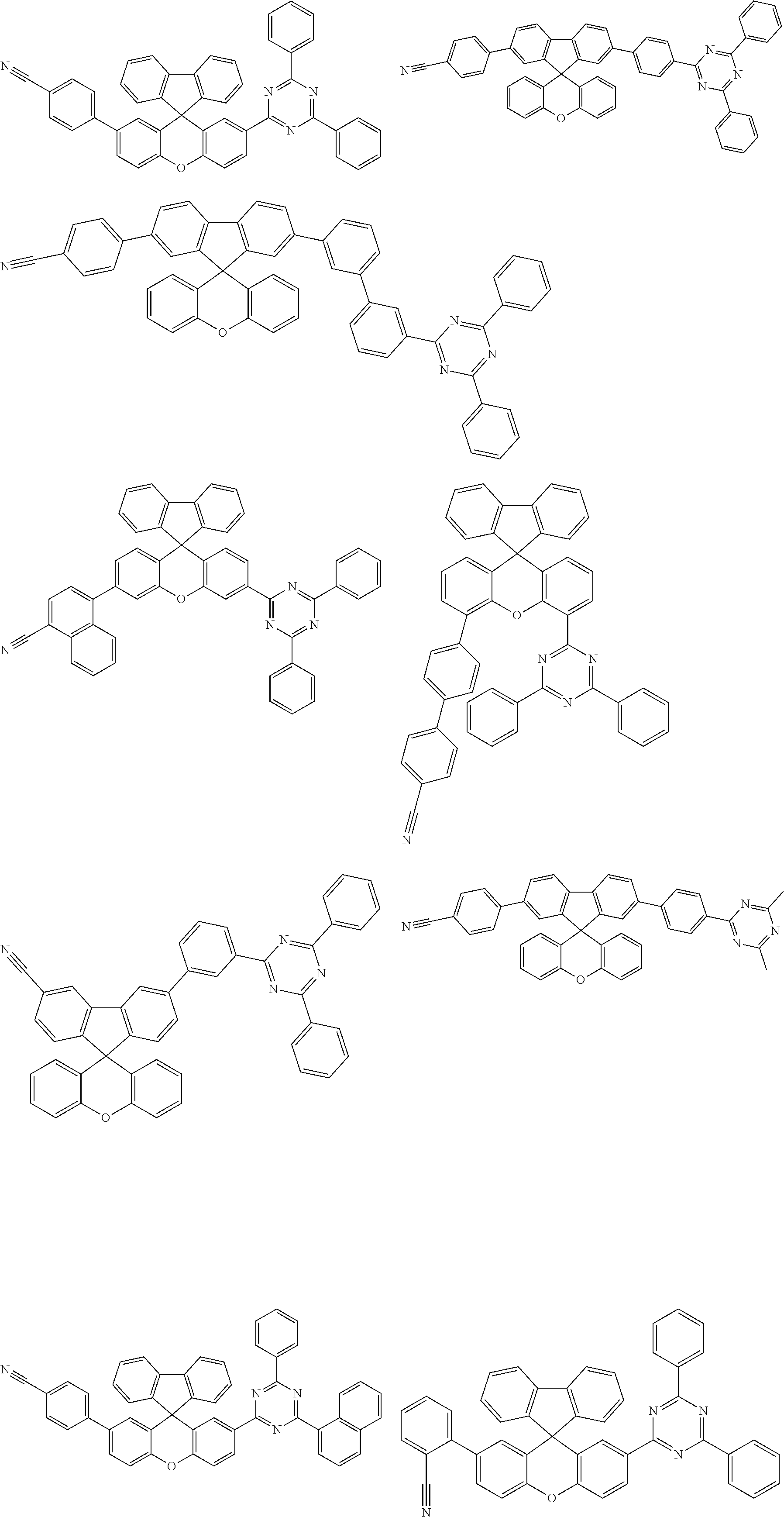

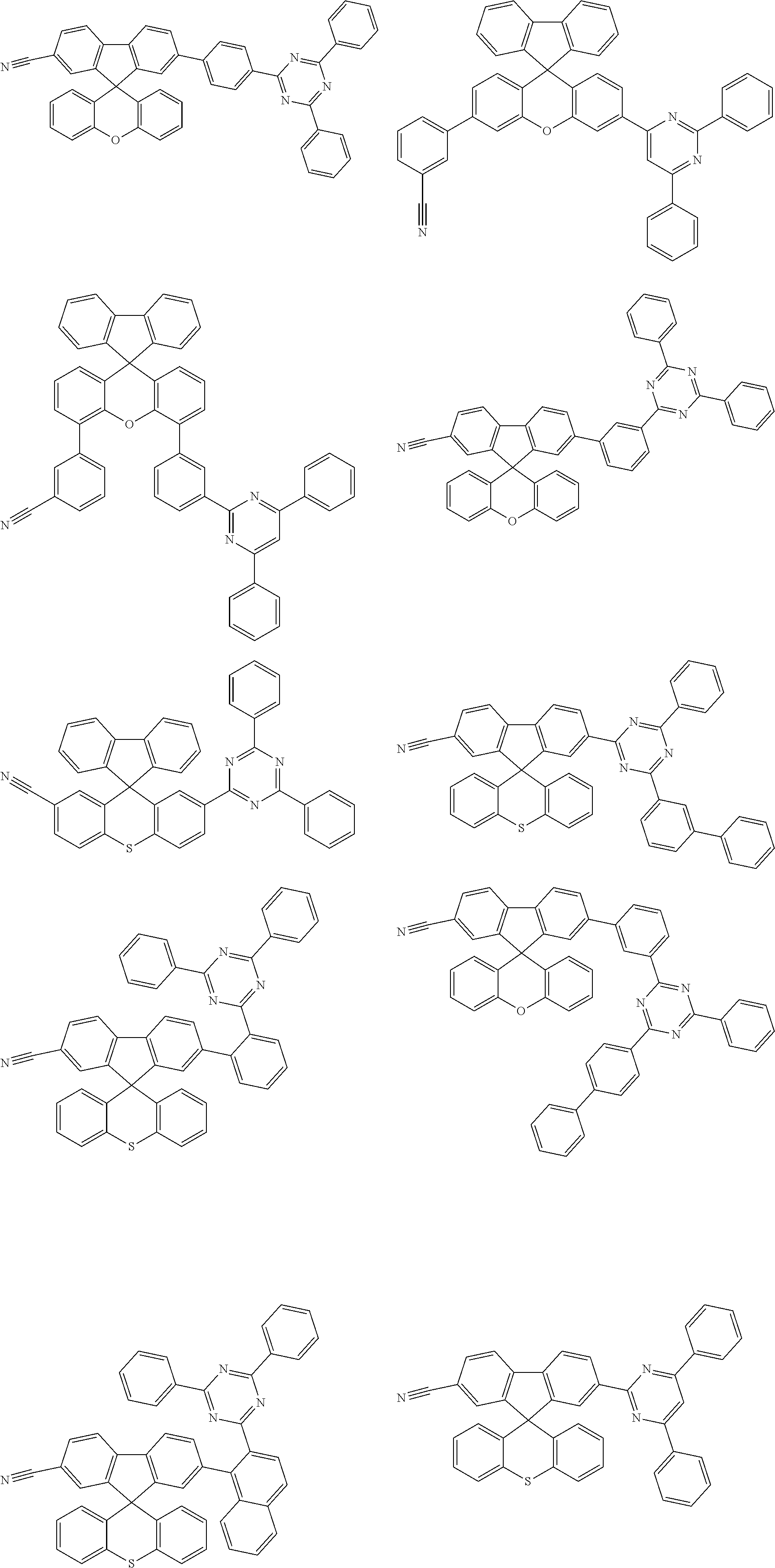

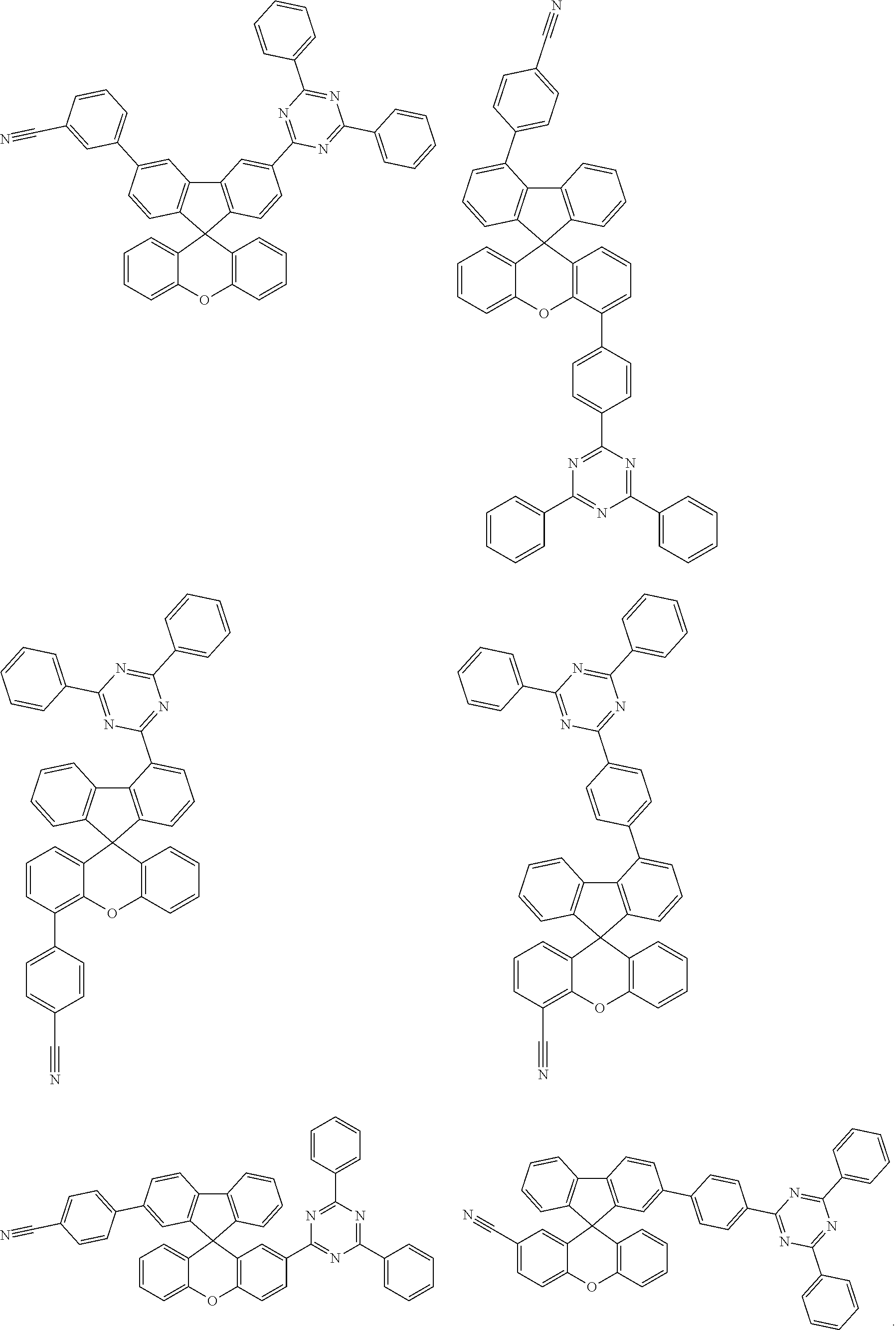

7. The organic light emitting device claim 1, comprising one or more organic material layers between the anode and the first organic material layer.

8. The organic light emitting device claim 1, wherein the first organic material layer is adjacent to the light emitting layer.

9. The organic light emitting device claim 1, comprising one or more organic material layers between the light emitting layer and the second organic material layer.

10. The organic light emitting device claim 1, wherein the light emitting layer further includes a dopant.
